(19) United States
(12) Patent Application Publication
(10) Pub. No.: US 2021/0346495 A1
(43) Pub. Date: Nov. 11, 2021

(54) METHODS AND COMPOSITIONS COMPRISING AN NFKB INHIBITOR AND AN ADJUVANT

(71) Applicant: THE UNIVERSITY OF CHICAGO, Chicago, IL (US)

(72) Inventors: Aaron ESSER-KAHN, Chicago, IL (US); Brittany MOSER, Chicago, IL (US)

(73) Assignee: THE UNIVERSITY OF CHICAGO, Chicago, IL (US)

(21) Appl. No.: 17/337,610

(22) Filed: Jun. 3, 2021

Related U.S. Application Data
(63) Continuation of application No. PCT/US2019/064888, filed on Dec. 6, 2019.
(60) Provisional application No. 62/776,860, filed on Dec. 7, 2018.
(60) Provisional application No. 62/924,315, filed on Oct. 22, 2019.

(51) Int. Cl. A61K 39/39; A61K 31/165; A61K 38/18; A61K 31/05; A61K 31/09; A61K 31/085; A61K 39/145; A61K 39/21; A61K 39/12; A61P 37/04; A61P 29/00; A61P 31/12

(52) U.S. Cl. CPC A61K 39/39; A61K 31/165; A61K 38/1825; A61K 31/05; A61K 31/09; A61K 31/085; A61K 2039/55561; A61K 39/21; A61K 39/12; A61P 37/04; A61P 29/00; A61P 31/12; A61K 39/145

(57) ABSTRACT

The current disclosure describes the use of NFkB inhibitors as immune potentiators in vaccine compositions comprising adjuvants. Accordingly, aspects of the disclosure relate to a method for vaccinating a subject comprising administering a NFkB inhibitor and an adjuvant (or a composition of the disclosure comprising NFkB and an adjuvant) to the subject. Further aspects relate to a method for inhibiting an inflammatory reaction associated with an adjuvant in a subject, the method comprising co-administering a NFkB inhibitor and an adjuvant (or a composition of the disclosure comprising NFkB and an adjuvant) to the subject. Yet further aspects relate to a pharmaceutical composition comprising a NFkB inhibitor and an adjuvant.

### CROSS-REFERENCE TO RELATED APPLICATIONS

This application is a continuation of PCT Patent Application No. PCT/US2019/064888, filed Dec. 6, 2019, which claims the benefit of priority of U.S. Provisional Patent Application No. 62/776,860, filed Dec. 7, 2018, and U.S. Provisional Patent Application No. 62/924,315, filed Oct. 22, 2019, all of which are hereby incorporated by reference in their entirety.

This invention was made with government support under AI124286, AI112194, GM099594 awarded by the National Institutes of Health and under DGE1321946 awarded by the National Science Foundation. The government has certain rights in the invention.

### BACKGROUND

### 1. Field of the Invention

The present invention relates generally to the field of prophylactic and therapeutic vaccines. More specifically, the invention relates to methods and compositions that increase the safety and effectiveness of vaccines.

### 2. Description of Related Art

Vaccines are considered one of the most effective global health interventions against infectious diseases. Despite their success, current and future vaccines face contradictory challenges of increasingly stringent safety margins and more effective and diverse protective responses. A major challenge in developing new vaccine approaches is striking a balance between effective immune activation, leading to protective responses, and limiting the excess inflammation and side effects. To boost the immune response, toll-like receptor (TLR) agonists have been explored as vaccine adjuvants because they activate the innate immune system, promoting the expression of inflammatory cytokines and cell surface receptors important for T cell interactions (1-6). Effective TLR agonists stimulate the desired cellular or humoral adaptive responses; however, the inflammation induced by many of these compounds has made it challenging to transition them into new clinical vaccines (7). For example, CpG DNA, a TLR 9 agonist, has wide-ranging promise as a vaccine adjuvant and provides protection for diseases currently without a vaccine, such as HIV (8). CpG DNA also enables vaccines to be produced with less antigen (9), induces protective responses faster (10), and produces effective anti-tumor activity (11,12). However, the excessive inflammatory response induced by this adjuvant has resulted in many clinical trial failures and is cited as limiting its therapeutic promise (13,14). CpGs are only a fraction of the hundreds of TLR agonists (15). However due to the unsafe side effects, only a handful of TLR agonists are approved for limited use in humans (16). Studies indicate that side effects are mediated through systemic distribution of TNF-a and IL-6 (17,18).

Accordingly, the current vaccination methods, particularly those that require adjuvants, may pose safety concerns, and there is a need in the art for strategies for increasing the safety of vaccines.

Here the inventors demonstrate a method to decouple part of the inflammatory response from the antigen presenting actions of several adjuvants using an immune potentiator. Using a broad range of TLR agonists, the inventors demonstrate both in vitro and in vivo that using an immune potentiator decreases proinflammatory cytokines while maintaining adaptive immune function. In vivo, the inventors find that co-administering the immune potentiator with the 2017-2018 flu vaccine (Fluzone) decreases side effects associated with vaccination and increases protection. Co-administration of the immune potentiator with CpG-ODN1826 (CpG) and dengue capsid protein leads to elimination of systemic proinflammatory cytokines post-vaccination and yields increased, neutralizing antibodies. Additionally, administering the immune potentiator with CpG and gp120, a HIV viral coat protein, increased serum IgG and vaginal IgA antibodies and shifted IgG antibody epitope recognition. Lastly, the inventors observed immune potentiation for several TLR agonists—implying a general approach. Immune-potentiation may find use in reducing the systemic side effects associated with inflammation for many adjuvanted vaccines (19)—creating the potential for many PRR agonists to be used safely, increasing the diversity of adaptive immune profiles and widening the scope of disease prevention and treatment.

### SUMMARY OF THE INVENTION

The current disclosure describes the use of NFkB inhibitors as immune potentiators in vaccine compositions comprising adjuvants. Accordingly, aspects of the disclosure relate to a method for vaccinating a subject comprising administering a NFkB inhibitor and an adjuvant (or a composition of the disclosure comprising NFkB and an adjuvant) to the subject. Further aspects relate to a method for inhibiting an inflammatory reaction associated with an adjuvant and potentiating an immune response in a subject, the method comprising co-administering a NFkB inhibitor and an adjuvant (or a composition of the disclosure comprising NFkB and an adjuvant) to the subject. Yet further aspects relate to a pharmaceutical composition comprising a NFkB inhibitor and an adjuvant.

In some embodiments, the NFkB inhibitor comprises SN50, capsaicin, withaferin A, parthenolide, luteolin, caffeic acid phenyl ester, 5z-7-oxozeaenol, a compound of formula (I)

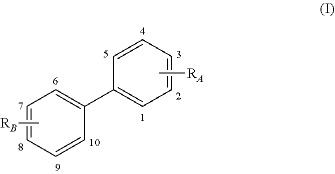

where R_{A} is attached to one or more ring atoms at positions 1, 2, 3, 4, and 5, and each R_{A} is independently hydrogen, hydroxyl, alkoxy, alkenoxy, or alkenyl, and R_{B} is attached to one or more ring atoms at positions 6, 7, 8, 9, and 10, and each R_{B} is independently hydrogen, hydroxyl, alkoxy, alkenoxy, or alkenyl, or combinations thereof. In some aspects, R_{A} is selected from hydrogen, hydroxyl, allyl, allyl ether, and vinyl ether. In some embodiments, R_{B} is selected from hydrogen, hydroxyl, allyl, allyl ether, and vinyl ether. In some embodiments, the NFkB inhibitor comprises cardamonin, caffeic acid phenethyl ester (CAPE), withaferin A (WA), resveratrol, salicin, 5Z-7-Oxozeaenol, parthenolide, honokiol, capsaicin, PDK1/Akt/Flt dual pathway inhibitor (PDK1), GYY 4137 (GYY), or combinations thereof. In some embodiments, the NFkB inhibitor comprises capsaicin. In some embodiments, NFkB inhibitor comprises comprises honokiol. In some embodiments, the NFkB inhibitor is at least one of

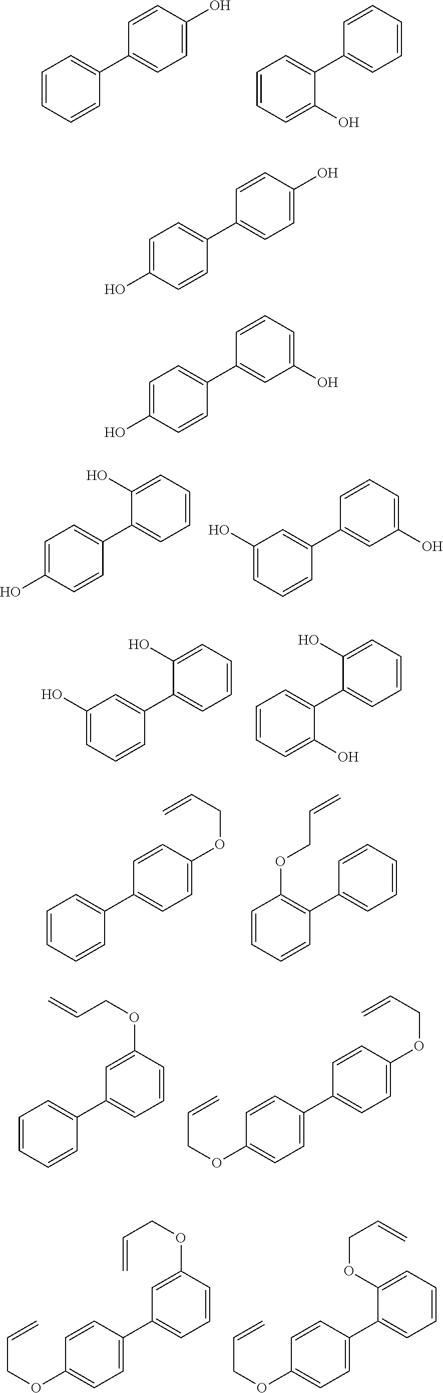

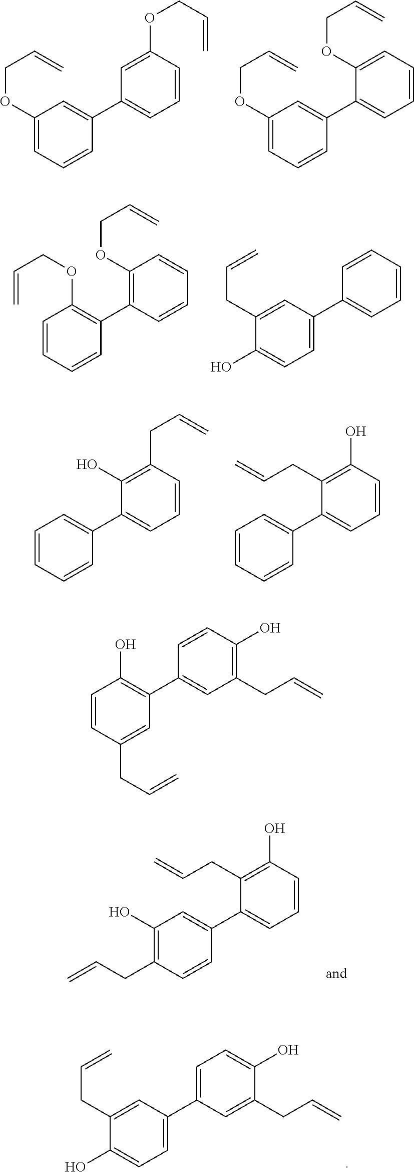

In some embodiments, the NFkB inhibitor comprises SN50. In some embodiments, the NFkB inhibitor excludes SN50. In some embodiments, the NFkB inhibitor comprises cardamonin, withaferin A, luteolin, bengamide B, IRAK1/4 inhibitor, histone acetyltransferase inhibitor II, parthenolide, capsaicin, MG132, PD 98059, Tp12 kinase inhibitor, curcumin, resveratrol, caffeic acide phenyl ester, honokiol, GYY, LY294002, IKKVII inhibitor, PDK1, TSA, JNK II inhibitor, (5Z)-7-Oxo Zeaenol, Salicin, Paenol, QNZ, IMD, IL-6 neutralizing antibody, or TNF-a neutralizing antibody. In some embodiments, one or more of cardamonin, withaferin A, luteolin, bengamide B, IRAK1/4 inhibitor, histone acetyltransferase inhibitor II, parthenolide, capsaicin, MG132, PD 98059, Tpl2 kinase inhibitor, curcumin, resveratrol, caffeic acide phenyl ester, honokiol, GYY, LY294002, IKKVII inhibitor, PDK1, TSA, JNK II inhibitor, (5Z)-7-Oxo Zeaenol, Salicin, Paenol, QNZ, IMD, IL-6 neutralizing antibody, a compound of formula (I), and TNF-a neutralizing antibody is specifically excluded.

In some embodiments, the adjuvant comprises a pattern recognition receptor (PRR). In some embodiments, the adjuvant comprises a toll-like receptor (TLR). In some embodiment, the adjuvant comprises CpG, AddaVax, R848, Pam3CSK4, or LPS. In some embodiments, the adjuvant comprises one or more adjuvants described herein. In some embodiments, the adjuvant comprises CpG, R848, Pam3CSK4, LPS, MPLA, Complete Freund's adjuvant, or Incomplete Freund's adjuvant. In some embodiments, the adjuvant excludes CpG, AddaVax, R848, Pam3CSK4, LPS, MPLA, Complete Freund's adjuvant, or Incomplete Freund's adjuvant.

In some embodiments, the composition comprises two or more naturally occurring compounds that are not found together in nature. In some embodiments, the composition provides an effect not seen with either component alone. For example, the composition, when administered to a subject, is effective in potentiating an immune response and/or attenuate an inflammatory response. In some embodiments, the compositions comprise one or more components, such as a NFkB inhibitor, an adjuvant, or an antigen, wherein the one or more components is a non-natural element.

In some embodiments, the NFkB inhibitor comprises SN50 and the TLR agonist comprises a TLR9 agonist. In some embodiments, the NFkB inhibitor comprises SN50 and the TLR agonist comprises CpG, R848, Pam3CSK4, or LPS.

In some embodiments, the NFkB inhibitor comprises SN50 and the adjuvant comprises R848. In some embodiments, the NFkB inhibitor comprises SN50 and the adjuvant comprises Pam3CSK4. In some embodiments, the NFkB inhibitor comprises SN50 and the adjuvant comprises AddaVax. In some embodiments, the NFkB inhibitor comprises SN50 and the adjuvant comprises LPS. In some embodiments, the NFkB inhibitor comprises Honokiol and the adjuvant comprises R848. In some embodiments, the NFkB inhibitor comprises Honokiol and the adjuvant comprises Pam3CSK4. In some embodiments, the NFkB inhibitor comprises Honokiol and the adjuvant comprises AddaVax. In some embodiments, the NFkB inhibitor comprises Honokiol and the adjuvant comprises LPS. In some embodiments, the NFkB inhibitor comprises Honokiol and the adjuvant comprises CpG. In some embodiments, the NFkB inhibitor comprises capsaicin and the adjuvant comprises R848. In some embodiments, the NFkB inhibitor comprises capsaicin and the adjuvant comprises Pam3CSK4. In some embodiments, the NFkB inhibitor comprises capsaicin and the adjuvant comprises AddaVax. In some embodiments, the NFkB inhibitor comprises capsaicin and the adjuvant comprises LPS. In some embodiments, the NFkB inhibitor comprises capsaicin and the adjuvant comprises CpG. In some embodiments, the NFkB inhibitor comprises a compound of formula (I) and the adjuvant comprises R848. In some embodiments, the NFkB inhibitor comprises a compound of formula (I) and the adjuvant comprises Pam3CSK4. In some embodiments, the NFkB inhibitor comprises a compound of formula (I) and the adjuvant comprises AddaVax. In some embodiments, the NFkB inhibitor comprises a compound of formula (I) and the adjuvant comprises LPS. In some embodiments, the NFkB inhibitor comprises withaferin A and the adjuvant comprises R848. In some embodiments, the NFkB inhibitor comprises withaferin A and the adjuvant comprises Pam3CSK4. In some embodiments, the NFkB inhibitor comprises withaferin A and the adjuvant comprises AddaVax. In some embodiments, the NFkB inhibitor comprises withaferin A and the adjuvant comprises LPS. In some embodiments, the NFkB inhibitor comprises withaferin A and the adjuvant comprises CpG. In some embodiments, the NFkB inhibitor comprises parthenolide and the adjuvant comprises R848. In some embodiments, the NFkB inhibitor comprises parthenolide and the adjuvant comprises Pam3CSK4. In some embodiments, the NFkB inhibitor comprises parthenolide and the adjuvant comprises AddaVax. In some embodiments, the NFkB inhibitor comprises parthenolide and the adjuvant comprises LPS. In some embodiments, the NFkB inhibitor comprises parthenolide and the adjuvant comprises CpG. In some embodiments, the NFkB inhibitor comprises luteolin and the adjuvant comprises R848. In some embodiments, the NFkB inhibitor comprises luteolin and the adjuvant comprises Pam3CSK4. In some embodiments, the NFkB inhibitor comprises luteolin and the adjuvant comprises AddaVax. In some embodiments, the NFkB inhibitor comprises luteolin and the adjuvant comprises LPS. In some embodiments, the NFkB inhibitor comprises luteolin and the adjuvant comprises CpG. In some embodiments, the NFkB inhibitor comprises caffeic acid phenyl ester and the adjuvant comprises R848. In some embodiments, the NFkB inhibitor comprises caffeic acid phenyl ester and the adjuvant comprises Pam3CSK4. In some embodiments, the NFkB inhibitor comprises caffeic acid phenyl ester and the adjuvant comprises AddaVax. In some embodiments, the NFkB inhibitor comprises caffeic acid phenyl ester and the adjuvant comprises LPS. In some embodiments, the NFkB inhibitor comprises caffeic acid phenyl ester and the adjuvant comprises CpG. In some embodiments, the NFkB inhibitor comprises 5z-7-oxozeaenol and the adjuvant comprises R848. In some embodiments, the NFkB inhibitor comprises 5z-7-oxozeaenol and the adjuvant comprises Pam3CSK4. In some embodiments, the NFkB inhibitor comprises 5z-7-oxozeaenol and the adjuvant comprises AddaVax. In some embodiments, the NFkB inhibitor comprises 5z-7-oxozeaenol and the adjuvant comprises LPS. In some embodiments, the NFkB inhibitor comprises 5z-7-oxozeaenol and the adjuvant comprises CpG.

In some embodiments, the adjuvant comprises CpG ODN 1018, MPLA, Pam3CSK4, Pam2CSK4, R848, 2BXy, QS-21, AS01B, Freund's complete adjuvant, or combinations thereof. In some embodiments, the adjuvant comprises CpG ODN 1018. In some embodiments, the adjuvant comprises MPLA. In some embodiments, the adjuvant comprises Pam3CSK4. In some embodiments, the adjuvant comprises Pam2CSK4. In some embodiments, the adjuvant comprises R848. In some embodiments, the adjuvant comprises 2BXy. In some embodiments, the adjuvant comprises QS-21. In some embodiments, the adjuvant comprises AS01B. In some embodiments, the adjuvant comprises Freund's complete adjuvant.

In some embodiments, the method further comprises administration of one or more antigens. In some embodiments, the method further comprises administration of inactivated virus, live attenuated virus, or antigenic fragments thereof. In some embodiments, the virus comprises influenza, dengue, or HIV. In some embodiments, the method comprises administration of a dengue antigen. In some embodiments, the antigen comprises capsid protein of dengue serotype-2 (DENV-2C). In some embodiments, the method comprises administration of a HIV antigen. In some embodiments, the HIV antigen comprises gp120. In some embodiments, the NFkB inhibitor is administered in combination with Fluzone®. In some embodiments, the NFkB inhibitor is administered in combination with a trivalent or quadrivalent flu vaccine.

In some embodiments, the NFkB inhibitor, antigen, and/or adjuvant is administered by intramucosal, intramuscular, parenteral, or subcutaneous administration. In some embodiments, the NFkB inhibitor is administered by a route of administration described herein. In some embodiments, the NFkB inhibitor is administered prior to administration of the adjuvant. In some embodiments, the NFkB inhibitor is administered prior to the antigen. In some embodiments, the NFkB inhibitor is administered after the adjuvant. In some embodiments, the NFkB inhibitor is administered after the antigen. In some embodiments, the NFkB inhibitor is administered at least or at most 0.5, 1, 2, 3, 4, 5, or 10 hours or 1, 2, 3, 4, 5, 6, 7, 8, 9, 10, 11, 12, 13, or 14, days before or after (or any derivable range therein) the adjuvant and/or antigen. In some embodiments, the NFkB inhibitor and the adjuvant are administered simultaneously. In some embodiments, the NFkB inhibitor and the antigen are administered simultaneously. In some embodiments, the NFkB inhibitor, adjuvant, and/or antigen are administered within 0.5, 1, 2, 3, 4, 5, 6, 7, 8, 9, or 10 hours of each other (or any range derivable therein). In some embodiments, NFkB inhibitor, the adjuvant, and/or the antigen are administered locally to the same site in the subject. In some embodiments, the NFkB inhibitor, the adjuvant, and/or the antigen are administered in the same composition to the subject. In some embodiments, the NFkB inhibitor is administered in a separate composition than the adjuvant and/or antigen.

In some embodiments, at least 12 mg of NFkB inhibitor are administered to the subject. In some embodiments, at least, at most, or about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 125, 150, 175, 200, 225, 250, 275, 300, 325, 350, 375, 400, 425, 450, 475, or 500 mg of NFkB inhibitor (or any derivable range therein) is administered to the subject. In some embodiments, 0.2 mg/kg NFkB is administered to the subject. In some embodiments, at least, at most, or about 0.01, 0.05, 0.07, 0.1, 0.15, 0.2, 0.25, 0.3, 0.35, 0.4, 0.45, 0.5, 0.55, 0.6, 0.65, 0.7, 0.75, 0.8, 0.85, 0.9. 1, 1.5, 2, 2.5, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg/kg (or any derivable range therein) of NFkB inhibitor is administered to the subject. In some embodiments, the amount of NFkB inhibitor administered to a human or non-human primate subject corresponds to a dose that is equal to or greater than 50 micrograms in a mouse. In some embodiments, the amount of NFkB inhibitor administered to a human or non-human primate subject corresponds to a dose that is more than or less than 1, 2, 3, 4, 5, 6, 7, 8, 9, 10, 20, 30, 40, 50, 60, 70, 80, 90, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, or 900 micrograms (or any derivable range therein) in a mouse.

In some embodiments, the subject is a human. In some embodiments, the subject is a non-human primate, a mouse, a goat, a rabbit, a dog, a horse, or a sheep.

In some embodiments, the method is for preventing a disease in the subject. In some embodiments, the method is for treating a disease in a subject.

In some embodiments, the subject has previously been administered an adjuvant. In some embodiments, the subject has not previously been administered an adjuvant. In some embodiments, the subject is one that has had an adverse reaction to a previous administration of an adjuvant or to a vaccine. In some embodiments, the adverse reaction comprises systemic inflammation.

Some aspects of the disclosure are directed to a pharmaceutical composition comprising a NFKB inhibitor. In some embodiments, the NFKB inhibitor is a compound of formula (I)

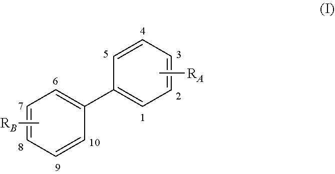

where R_{A} is attached to one or more ring atoms at positions 1, 2, 3, 4, and 5, and each R_{A} is independently hydrogen, hydroxyl, alkoxy, alkenoxy, or alkenyl, and R_{B} is attached to one or more ring atoms at positions 6, 7, 8, 9, and 10, and each R_{B} is independently hydrogen, hydroxyl, alkoxy, alkenoxy, or alkenyl. In some aspects, R_{A} is selected from hydrogen, hydroxyl, allyl, allyl ether, and vinyl ether. In some embodiments, R_{B} is selected from hydrogen, hydroxyl, allyl, allyl ether, and vinyl ether. In some embodiments, the NFKB inhibitor is at least one of

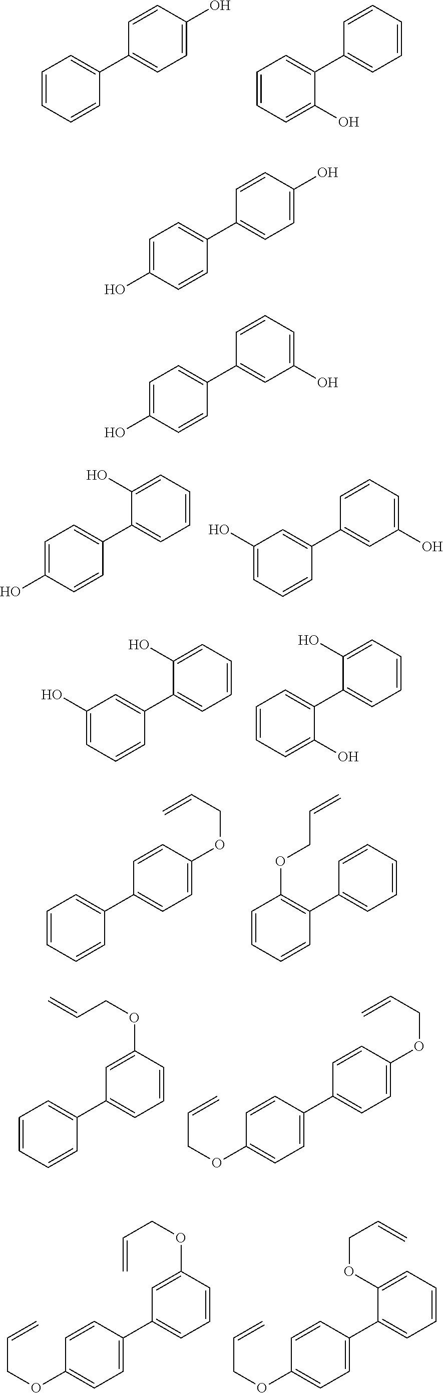

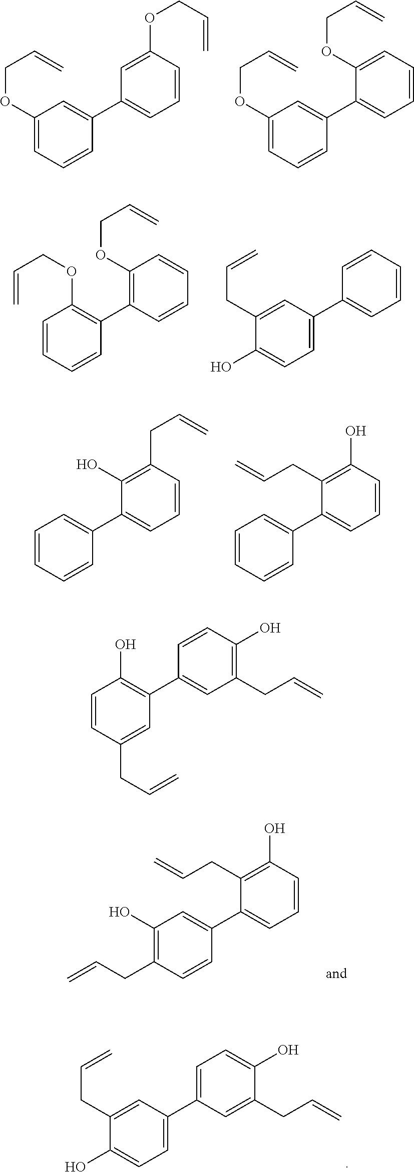

In some embodiments, the composition is formulated for intramucosal, intramuscular, parenteral, or subcutaneous administration. In some embodiments, the composition further comprises a pharmaceutical excipient.

The methods and compositions of the disclosure may be used to reduce systemic inflammation, such as that associated with vaccination and/or vaccines comprising an adjuvant. In some embodiments, the methods of the disclosure reduce adjuvant-induced inflammation while also increasing the adaptive immune response. In some embodiments, the methods of the disclosure reduce one or both of IL-6 and TNF-α. The methods and compositions of the disclosure may be used to enhance antigen presentation and T cell activation, and/or increase antibody titer. The methods and compositions of the disclosure may also enhance epitope selectivity, shift epitope selectivity, and/or provide for a vaccine that produces a broad-spectrum antibody response.

The preparation of the vaccine as the active immunogenic ingredient, may be prepared as injectables, either as liquid solutions or suspensions; solid forms suitable for solution in, or suspension in, liquid prior to infection can also be prepared. The preparation may be emulsified, encapsulated in liposomes. The active immunogenic ingredients are often mixed with carriers which are pharmaceutically acceptable and compatible with the active ingredient.

Administration of vaccines according to the disclosure may be via any common route so long as the target tissue is available via that route in order to maximize the delivery of antigen to a site for maximum (or in some cases minimum) immune response. Administration will generally be by orthotopic, intradermal, mucosally, subcutaneous, intramuscular, intraperitoneal or intravenous injection. Other areas for delivery include: oral, nasal, buccal, rectal, vaginal or topical. Vaccines of the invention are preferably administered parenterally, by injection, for example, either subcutaneously or intramuscularly.

Vaccines may be administered in a manner compatible with the dosage formulation, and in such amount as will be prophylactically and/or therapeutically effective. The quantity to be administered depends on the subject to be treated, including, e.g., capacity of the subject's immune system to synthesize antibodies, and the degree of protection or treatment desired. Suitable dosage ranges are of the order of several hundred micrograms active ingredient per vaccination with a range from about 0.1 mg to 1000 mg, such as in the range from about 1 mg to 300 mg, or in the range from about 10 mg to 50 mg. Suitable regimens for initial administration and booster shots are also variable but are typified by an initial administration followed by subsequent inoculations or other administrations. Precise amounts of active ingredient required to be administered depend on the judgment of the practitioner and may be peculiar to each subject. It will be apparent to those of skill in the art that the therapeutically effective amount of nucleic acid molecule or fusion polypeptides of this invention will depend, inter alia, upon the administration schedule, the unit dose of antigen administered, whether the vaccine composition is administered in combination with other therapeutic agents, and the immune status and health of the recipient.

A vaccine may be given in a single dose schedule or in a multiple dose schedule. A multiple dose schedule is one in which a primary course of vaccination may include, e.g., 1-10 separate doses, followed by other doses given at subsequent time intervals required to maintain and/or reinforce the immune response, for example, at 1-4 months for a second dose, and if needed, a subsequent dose(s) after several months. Periodic boosters at intervals of 1-5 years, usually 3 years, are desirable to maintain the desired levels of protective immunity.

A vaccine may be provided in one or more “unit doses”. Unit dose is defined as containing a predetermined-quantity of the vaccine calculated to produce the desired responses in association with its administration, i.e., the appropriate route and treatment regimen. The quantity to be administered, and the particular route and formulation, are within the skill of those in the clinical arts. The subject to be treated may also be evaluated, in particular, the state of the subject's immune system and the protection desired. A unit dose need not be administered as a single injection but may include continuous infusion over a set period of time. Unit dose of the present invention conveniently may be described in terms of mg/kg body weight. In some embodiments, the dose of the NFkB inhibitor, adjuvant, or antigen may be at least, at most, or about 0.05, 0.10, 0.15, 0.20, 0.25, 0.5, 1, 10, 50, 100, 1,000 or any derivable range therein mg/kg. Likewise the amount of vaccine delivered can vary from about 0.2 to about 8.0 mg/kg body weight. Thus, in particular embodiments, 0.1 mg/kg, 0.2 mg/kg, 0.3 mg/kg, 0.4 mg/kg, 0.5 mg/kg, 0.8 mg/kg, 1.0 mg/kg, 1.5 mg/kg, 2.0 mg/kg, 2.5 mg/kg, 3.0 mg/kg, 4.0 mg/kg, 5.0 mg/kg, 5.5 mg/kg, 6.0 mg/kg, 6.5 mg/kg, 7.0 mg/kg and 7.5 mg/kg (or any derivable range therein) of the vaccine may be delivered to an individual in vivo. The dosage of vaccine to be administered depends to a great extent on the weight and physical condition of the subject being treated as well as the route of administration and the frequency of treatment.

In some embodiments, the methods of the disclosure comprise administering one or more compositions two or more times. It is contemplated that the compositions may be administered 1, 2, 3, 4, 5, 6, 7, 8, 9, 10, 11, 12, 13 or 14 days apart or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51 or 52 weeks apart or 1, 2, 3, 4, 5, 6, 7, 8, 9, 10, 11, 12, 13, 14, 15, 16, 17, 18, 19, 20, 21, 22, 23, 24, 36, 48, 60, 72, 84 or 96 months apart or 1, 2, 3, 4, 5, 6, 7, 8, 9, 10, 11, 12, 13, 14, 15, 16, 17, 18, 19 or 20 years apart (or any derivable range therein).

Further aspects relate to a kit comprising compositions of the disclosure and instructions for use.

In some embodiments, methods further comprise testing the patient for an infection, such as a viral infection or diagnosing a patient with an infection, such as a viral infection.

Any embodiment discussed in the context of an antibody may be implemented in any method embodiment discussed herein.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: In vivo vaccination with model antigen ovalbumin and immune adjuvant SN50. (A) Intracellular cytokine staining of BMDCs treated with CpG (red bars) or CpG+SN50 (blue bars). (B) Systemic cytokine levels of TNF-α measured at 1 h, 3 h, 6 h post-injection with: PBS (black line), SN50 alone (purple line), CpG (red line), CpG+SN50 (blue line), CpG+SN50M (yellow line), n=4 for each time point. (C) Systemic cytokine levels of IL-6. (D) Anti-ovalbumin antibody titer, day 28, n=8. (E) Systemic TNF-α levels 1 h post-vaccination with CpG, CpG+IL-6N, CpG+TNF-αN or CpG+Control ab, n=4. (F) Systemic IL-6 levels 1 h post-vaccination with CpG, CpG+IL-6N, CpG+TNF-αN or CpG+Control ab. (G) Anti-ovalbumin antibody titer, day 28. (H) Systemic TNF-α levels in mice vaccinated with mixed CpG and SN50 (CpG+SN50), SN50 in left limb and CpG+OVA in right limb (SN50L, CpG R, or CpG alone, n=3. (I) Systemic IL-6 levels. (J) Anti-OVA antibody titer, day 28.

FIG. 2: Influenza Challenge Model. (A) Schematic of influenza challenge study. (B) Systemic TNF-α levels 1 h post-vaccination with Fz, Fz+SN50, Fz+CpG, Fz+CpG+SN50 H, Fz+CpG+SN50 L, Placebo. n=13 (C) Systemic IL-6 levels 1 h post-vaccination. n=13 (D) Percent change in body weight 24 h (grey), 48 h (blue) and 72 h (green) post-vaccination, n=13. (E) CD4+ IL4+ splenocytes after antigen restimulation n=5. (F) CD8+ IFN-y+ splenocytes after antigen restimulation n=5. (G) Day 28 IgG antibody titer, n=8. (H) Survival 1-14 days post challenge, n=5. Groups: Fz (black), Fz+SN50 (blue), Fz+CpG (grey), Fz+CpG+SN50 H (red), Fz+CpG+SN50 L (purple), Placebo (orange). n=5 (I) Percent change in body weight 1-14 days, n=5. (J) Body temperature 1-14 days post challenge, n=5. (K) Safety vs Protection score.

FIG. 3: In vivo vaccination against dengue and HIV. (A) Systemic TNF-α levels 1 h post-vaccination with DENV-2C antigen and CpG or CpG+SN50, n=6. (B) Systemic IL-6 levels 1 h post-vaccination with DENV-2C antigen and CpG or CpG+SN50. (C) IgG antibody titer day 28 post vaccination with DENV-2C antigen. (D) Dengue virus neutralization. Geometric mean [95% confidence interval]. (E) Systemic TNF-α levels measured at 1 h post-injection with gp120 and: PBS, CpG, SN50, SN50+CpG, n=8 (F) Systemic IL-6 levels measured at 1 h post-injection with gp120 vaccinations (G) Serum anti-gp120 IgG antibody titer, day 28 after vaccination with gp120. (H) Vaginal anti-gp120 IgG antibody titer, day 28. (I) Serum anti-gp120 IgA antibody titer, day 28. (J) Vaginal anti-gp120 IgA antibody titer, day 28. (K) Number of g120 epitopes recognized by mice vaccinated with CpG or SN50+CpG. (L) Mean intensity of recognized epitopes. (M) Mean intensity of each recognized epitope by CpG (red bars) or CpG+SN50 (blue bars).

FIG. 4: In vivo vaccinations across a broad range of adjuvants. (A) qPCR gene expression analysis of RAW macrophages stimulated with SN50 and TLR agonists compared to cells stimulated with TLR agonist alone. Pro-inflammatory cytokines TNF-α (grey bars) and IL-6 (orange bars) and cell surface receptors CD40 (purple bars), CD80 (green bars), CD86 (red bars) and MHCII (blue bars). (B) Systemic TNF-a cytokine levels of TNF-a measured at 1 h post-injection with gp120 and: PBS, CpG, CpG+SN50, Pam3CSK4, Pam3CSK4+SN50, R848, R848+SN50, Alum, Alum+SN50, n=4. (C) Systemic IL-6 cytokine levels measured at 1 h post-injection. (D) Serum IgG antibody titers, day 28. (E) Human THP-1 cell pro-inflammatory cytokines TNF-α and IL-6 in cell supernatant after treatment with PBS (black bars), SN50 (orange bars), LPS (grey bars), or LPS+SN50 (blue bars). (F) Cell surface receptor expression on human THP-1 cell after treatment with PBS (black bars), SN50 (orange bars), LPS (grey bars), or LPS+SN50 (blue bars). (G) Cytokine expression analysis of TNF-α and IL-6 in cell supernatant of NHP PBMCs 6 h. No SN50 (red bars), SN50 (blue bars). LPS 1 μg/mL (H) CD86 expression of NHP PBMCs 18 h. No SN50 (red bars), SN50 (blue bars).

FIG. 5: NF-kB activity in mouse and human cells. (A) NF-kB activity in RAW blue macrophages stimulated with various TLR agonists and SN50 (blue bars), TLR agonists alone (grey bars). (B) RAW blue NF-kB activity of cells stimulated with SN50 (blue bars) and the control peptide SN50M (grey bars). (C) THP-1 NF-kB activity of cells stimulated with no peptide (grey bars), SN50 (blue bars) or SN50M (orange bars). (D) Concentration screen of 100 ng/mL LPS and various concentrations of SN50 (blue line) and SN50M (orange line).

FIG. 6: Proinflammatory Cytokine Analysis Time Course. (A) Systemic TNF-α levels measured at 1 h, 3 h, 6 h, 24, 48 h post vaccination. (B) Systemic IL-6 levels.

FIG. 7: SEM image of OVA vaccinations. Shown (from left to right) are CpG+OVA; SN50+OVA; CpG+SN50+OVA; and CpG+SN50M+OVA. Scale bar 2 um.

FIG. 8: Weight Loss Post-Vaccination. Weight loss 24 h (black dot), 48 h (blue dot) and 72 h (green dot) post prime. Fz=Fluzone 2017-2018 flu vaccine.

FIG. 9: Day 28 Antibody Titers. Fz=Fluzone 2017-2018 flu vaccine.

FIG. 10: Day 46 Antibody Titers (d3 post infection). Fz=Fluzone 2017-2018 flu vaccine.

FIG. 11: Day 57 Antibody Titers of surviving mice (d14 post infection). Fz (2), Fz+SN50 (5), Fz+CpG (5), Fz+CpG+SN50 Hi (5), Fz+CpG+SN50 Lo (5). Fz=Fluzone 2017-2018 flu vaccine.

FIG. 12: Lung viral titer d3 post-infection. Fz=Fluzone 2017-2018 flu vaccine.

FIG. 13: Full temperature curve for 14 days post-challenge. Fz (black line), Fz+SN50 (blue line), Fz+CpG (grey line), Fz+CpG+SN50 Hi (red line), Fz+CpG+SN50 Lo (purple line), Placebo (yellow line).

FIG. 14: IL-6 expression of RAW macrophages with LPS and small molecule NF-kB inhibitors. CARD=Cardamonin, WA=Withaferin A, HA=histone acetylase inhibitor, 5-z-o=5z-7-oxozeaenol.

FIG. 15: In vivo study of NF-kB inhibitors with CpG.

FIG. 16: In vivo study of NF-kB inhibitors with AddaVax and CpG or Pam3CSK4 with NF-kB inhibitors. WA=withaferin A

FIG. 17: In vivo dosage analysis of capsaicin and honokiol with CpG and AddaVax.

FIG. 18: Small molecule inhibitor screen in vitro and in vivo. (A) IL-6 levels from RAW macrophages 24 h post-stimulation with NF-kB inhibitor and LPS. Significance is compared to LPS alone. * p<0.05, **p<0.01, p<0.001. (B) Systemic TNF-α expression 1 h post-vaccination. (C) Systemic IL-6 expression 1 h post-vaccination. (D) Anti-OVA antibody level 21 days post-vaccination. Significance is compared to CpG vaccination. * p<0.05, **p<0.01, p<0.001.

FIG. 19: Broader cytokine response and dose effects of honokiol and capsaicin. (A-F) Systemic cytokine levels at 1 h, 24 h and 48 h post-vaccination. CpG (black line), CpG+Capsaicin (red line), CpG+Honokiol (blue line), PBS (purple line). (G) Systemic TNF-α levels 1 h post-vaccination with varying doses of honokiol and capsaicin. (H) Systemic IL-6 levels 1 h post-vaccination. (I) Anti-OVA antibody levels 21 days post-vaccination. Significance is compared to CpG alone. * p<0.05, **p<0.01, p<0.001.

FIG. 20: Role of TRPV1 of capsaicin induced anti-inflammatory and immune potentiation. (A) Systemic TNF-α levels 1 h post vaccination in wild type (WT) mice and TRPV1 KO (KO). (B) Systemic IL-6 levels 1 h post-vaccination. (C) Anti-OVA antibody level 21 days post-vaccination. * p<0.05, **p<0.01, p<0.001.

FIG. 21: Scheme employed for synthesis of honokiol derivatives.

FIG. 22: Honokiol and synthesized honokiol derivative library.

FIG. 23: Honokiol derivatives and their inhibitory activity on IL-6 expression. IL-6 expression of RAW macrophages treated with honokiol derivatives and LPS. Significance is compared to LPS alone. * p<0.05, **p<0.01, p<0.001.

FIG. 24: Capsaicin and PBS alone vaccinations in wild type and TRPV1 KO mice.

FIG. 25: Cell viability of RAW macrophages treated with honokiol derivative library.

### DESCRIPTION OF ILLUSTRATIVE EMBODIMENTS

Many modern vaccines include adjuvants that activate the immune system and provide an enhanced humoral or cellular response. Current approved adjuvants are unable to provide desired responses against some pathogens (e.g. HIV or dengue). Many new adjuvants have been developed and demonstrate promising results, but side effects from the inflammatory response induced by these adjuvants have resulted in limited FDA approvals. No adjuvants yet possess the capability to independently modulate inflammation and protection. The current disclosure provides for methods and compositions comprising a NF-kB inhibitor in combination with an adjuvant that can limit inflammation and side effects associated with vaccination while retaining the protective responses using a variety of adjuvants. The resulting vaccines reduce systemic inflammation and boosts antibody responses. In an influenza challenge model, this method was demonstrated in the Examples to enhance protection. This method is generalizable across a broad range of adjuvants and antigens.

### I. DEFINITIONS

The term “adjuvant” as used herein refers to substances, which when administered prior, together or after administration of an antigen, accelerate, prolong and/or enhance the quality and/or strength of an immune response to the antigen in comparison to the administration of the antigen alone.

As used herein, the term “vaccine” is intended to mean a composition which can be administered to humans or to animals in order to induce an immune system response; this immune system response can result in a production of antibodies or simply in the activation of certain cells, in particular antigen-presenting cells, T lymphocytes and B lymphocytes. In certain embodiments the vaccine is capable of producing an immune response that leads to the production of neutralizing antibodies in the patient with respect to the antigen provided in the vaccine. The vaccine can be a composition for prophylactic purposes or for therapeutic purposes, or both.

As used herein, the term “antigen” refers to any antigen that can be used in a vaccine, whether it involves a whole microorganism or a portion thereof, and various types: (e.g., peptide, protein, glycoprotein, polysaccharide, glycolipid, lipopeptide, etc). Thus, the term “antigen” refers to a molecule that can initiate a humoral and/or cellular immune response in a recipient of the antigen. In specific embodiments, the antigen is a molecule that causes a disease for which a vaccination would be advantageous treatment. In some embodiments, the antigen comprises a substance used to stimulate the production of antibodies and provide immunity against one or several diseases, prepared from the causative agent of a disease, its products, or a synthetic substitute, treated to act as an antigen without inducing the disease. In some embodiments, the antigen comprises a peptide or polypeptide.

As used herein, the term “attenuated recombinant virus” refers to a virus that has been genetically altered by modern molecular biological methods, e. g. restriction endonuclease and ligase treatment, and rendered less virulent than wild type, typically by deletion of specific genes or by serial passage in a non-natural host cell line or at cold temperatures.

The term “pharmaceutically acceptable carrier” refers to a carrier that does not cause an allergic reaction or other untoward effect in subjects to whom it is administered. Suitable pharmaceutically acceptable carriers include, for example, one or more of water, saline, phosphate buffered saline, dextrose, glycerol, ethanol, or the like and combinations thereof. In addition, if desired, the vaccine can contain minor amounts of auxiliary substances such as wetting or emulsifying agents, and pH buffering agents.

The term “aryl” includes heteroatom-unsubstituted aryl, heteroatom-substituted aryl, heteroatom-unsubstituted Cn-aryl, heteroatom-substituted Cn-aryl, heteroaryl, heterocyclic aryl groups, carbocyclic aryl groups, biaryl groups, and single-valent radicals derived from polycyclic fused hydrocarbons (PAHs). The term “heteroatom-unsubstituted Cn-aryl” refers to a radical, having a single carbon atom as a point of attachment, wherein the carbon atom is part of an aromatic ring structure containing only carbon atoms, further having a total of n carbon atoms, 5 or more hydrogen atoms, and no heteroatoms. For example, a heteroatom-unsubstituted C6-C10-aryl has 6 to 10 carbon atoms. Non-limiting examples of heteroatom-unsubstituted aryl groups include phenyl (Ph), methylphenyl, (dimethyl)phenyl, —C₆H₄CH₂CH₃, —C₆H₄CH₂CH₂CH₃, —C₆H₄CH(CH₃)₂, —C₆H₄CH(CH₂)₂, —C₆H₃(CH₃)CH₂CH₃, —C₆H₄CH═CH₂, —C₆H₄CH═CHCH₃, —C₆H₄C≡CH, —C₆H₄C≡CCH₃, naphthyl, and the radical derived from biphenyl. The term “heteroatom-substituted Cn-aryl” refers to a radical, having either a single aromatic carbon atom or a single aromatic heteroatom as the point of attachment, further having a total of n carbon atoms, at least one hydrogen atom, and at least one heteroatom, further wherein each heteroatom is independently selected from the group consisting of N, O, F, Cl, Br, I, Si, P, and S. For example, a heteroatom-unsubstituted C1-C10-heteroaryl has 1 to 10 carbon atoms. Non-limiting examples of heteroatom-substituted aryl groups include the groups: —C₆H₄F, —C₆H₄Cl, —C₆H₄Br, —C₆H₄I, —C₆H₄OH, —C₆H₄OCH₃, —C₆H₄OCH₂CH₃, —C₆H₄OC(O)CH₃, —C₆H₄NH₂, —C₆H₄NHCH₃, —C₆H₄N(CH₃)₂, —C₆H₄CH₂OH, —C₆H₄CH₂OC(O)CH₃, —C₆H₄CH₂NH₂, —C₆H₄CF₃, —C₆H₄CN, —C₆H₄CHO, —C₆H₄CHO, —C₆H₄C(O)CH₃, —C₆H₄C(O)C₆H₅, —C₆H₄CO₂H, —C₆H₄CO₂CH₃, —C₆H₄CONH₂, —C₆H₄CONHCH₃, —C₆H₄CON(CH₃)₂, furanyl, thienyl, pyridyl, pyrrolyl, pyrimidyl, pyrazinyl, quinolyl, indolyl, and imidazoyl. In certain embodiments, heteroatom-substituted aryl groups are contemplated. In certain embodiments, heteroatom-unsubstituted aryl groups are contemplated. In certain embodiments, an aryl group may be mono-, di-, tri-, tetra- or penta-substituted with one or more heteroatom-containing substituents.

The term “alkoxy” includes straight-chain alkoxy, branched-chain alkoxy, cycloalkoxy, cyclic alkoxy, heteroatom-unsubstituted alkoxy, heteroatom-substituted alkoxy, heteroatom-unsubstituted Cn-alkoxy, and heteroatom-substituted Cn-alkoxy. In certain embodiments, lower alkoxys are contemplated. The term “lower alkoxy” refers to alkoxys of 1-6 carbon atoms (that is, 1, 2, 3, 4, 5 or 6 carbon atoms). The term “heteroatom-unsubstituted Cn-alkoxy” refers to a group, having the structure —OR, in which R is a heteroatom-unsubstituted Cn-alkyl, as that term is defined above. Heteroatom-unsubstituted alkoxy groups include: —OCH₃, —OCH₂CH₃, —OCH₂CH₂CH₃, —OCH(CH₃)₂, and —OCH(CH₂)₂. The term “heteroatom-substituted Cn-alkoxy” refers to a group, having the structure —OR, in which R is a heteroatom-substituted Cn-alkyl, as that term is defined above. For example, —OCH₂CF₃ is a heteroatom-substituted alkoxy group.

The term “alkenyl” includes straight and branched chain hydrocarbon radicals containing one double bond and having from 2 to 6 carbon atoms such as, for example, ethenyl, 2-propenyl (allyl), 3-butenyl, 2-pentenyl, 3-pentenyl, 3-methyl-2-butenyl, and the like. The term “alkenoxy” refers to an alkenyl ether radical, where alkenyl is defined as above

The term “allyl” refers to the radical H₂C═CH—CH₂. The term “ether” refers to a hydrocarbyl group that is attached to another hydrocarbyl group via oxygen. Thus, the ether substituent of the hydrocarbyl group can be hydrocarbyl-O—. The ether can be symmetric or asymmetric. Examples of ethers include, but are not limited to vinyl ether and allyl ether. The term “vinyl”—refers to the portion of a molecule that includes a carbon-carbon double bond.

Various groups described herein, including hydroxyl, aryl, alkenoxy, alkoxy, and alkenyl, may be optionally substituted with one or more substituents. Non-limiting examples of substituent groups include halogen, nitro, cyano, hydroxy, amino, mercapto, formyl, carboxy, carbamoyl, alkyl, heteroalkyl, alkoxy, alkylthio, alkylamino, (alkyl)₂amino, alkylsulfinyl, alkylsulfonyl, arylsulfonyl, cycloalkyl, heterocyclyl, aryl, and heteroaryl.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

The term consisting essentially of may include the listed active ingredients, such as the recited antigen, adjuvant, and/or NFkB inhibitor, and also any unrecited buffers, pharmaceutical excipients, etc. but exclude any other active ingredients, such as other antigens, adjuvants, and/or NFkB inhibitors.

### II. NFKB INHIBITORS

NFkB refers to nuclear factor kappa-light-chain-enhancer of activated B cells. The table below provides NFkB inhibitors useful in the methods and compositions of the disclosure.

| Molecule | Type of molecule | Point of inhibition |
|---|---|---|
| Calagualine (fern derivative) | Natural product | Upstream of IKK (TRAF2-NIK) |
| Conophylline (Ervatamia microphylla) | Natural product | Down regulated TNF-Receptors |
| Evodiamine (Evodiae fructus component) | Natural product | AKT-IKK interaction |
| Geldanamycin | Natural product | IKK complex formation |
| Perrilyl alcohol | Natural product | Calcium pathway |
| Protein-bound polysaccharide from | Natural product | LPS-CD14 interaction |
| basidiomycetes | | |
| Rocaglamides (Aglaia derivatives) | Natural product | Upstream of IKK |
| 15-deoxy-prostaglandin J(2) | Natural product | PPARγ activation of NF-κB |
| Adenovirus E1A | Protein, viral | Adaptor |
| NS5A (Hepatitis C virus) | Protein, viral | TRAF2 inhibition |
| NS3/4A (HCV protease) | Protein | Upstream of IKK |
| Golli BG21 (product of myelin basic | Protein | Upstream of IKK (PKC) |
| protein) | | |
| NPM-ALK oncoprotein | Protein | Traf2 inhibition |
| MAST205 | Protein | TRAF6 binding |
| Erbin overexpression | Protein | NOD2 inhibitor |
| Rituximab (anti-CD20 antibody) | Protein | Up-regulates Raf-1 kinase inhibitor |
| Kinase suppressor of ras (KSR2) | Protein | MEKK3 inhibitor |
| PEDF (pigment epithelium derived factor) | Protein | ROS generation |
| TNAP | Protein | NIK |
| Betaine | Synthetic | NIK/IKK |
| Desloratadine | Synthetic | Histamine H1 receptor |
| LY29 and LY30 | Synthetic | PI3 Kinase inhibitors |
| MOL 294 (small molecule) | Synthetic | Redox regulated activation of NF-KB |
| Pefabloc (serine protease inhibitor) | Synthetic | Upstream of IKK |
| Rhein | Synthetic | MEKK activation of NF-κB |
| Salmeterol, fluticasone propionate | Synthetic | β2 agonists |
| Lead | Inorganic Complex | IKK activity |
| Anandamide | Natural product | IKKβ activity |
| Artemisia vestita | Natural product | Phosphorylation |
| Cobrotoxin | Natural product | IKKβ activity and p50 DNA binding |
| Dehydroascorbic acid (Vitamin C) | Natural product | IKKβ activity |
| Herbimycin A | Natural product | IKKβ activity |
| Isorhapontigenin | Natural product | IKKβ activity |
| Manumycin A | Natural product | IKKβ activity |
| Pomegranate fruit extract | Natural product | IKKα activity |
| Tetrandine (plant alkaloid) | Natural product | IKKα activity |
| Nitric oxide | Natural product | IKKβ activity /IκB phosphorylation |
| Thienopyridine | Natural product | IKKβ activity |
| Acetyl-boswellic acids | Natural product | IKK activity |
| β-carboline | Natural product | IKK activity |
| 1′-Acetoxychavicol acetate (Languas | Natural product | IKK activity |
| galanga) | | |
| Apigenin (plant flavinoid) | Natural product | IKK activity |
| Cardamomin | Natural product | IKK activity |
| Diosgenin | Natural product | IKK activity |
| Furonaphthoquinone | Natural product | IKK activity |
| Guggulsterone | Natural product | IKK activity |
| Falcarindol | Natural product | IKK activity |
| Honokiol | Natural product | IKK activity |
| Hypoestoxide | Natural product | IKK activity |
| Garcinone B | Natural product | IKK activity |
| Kahweol | Natural product | IKK activity |
| Kava (Piper methysticum) derivatives | Natural product | IKK activity |
| γ-mangostin (from Garcinia mangostana) | Natural product | IKK activity |
| N-acetylcysteine | Natural product | IKK activity |
| Nitrosylcobalamin (vitamin B12 analog) | Natural product | IKK activity |
| Piceatannol | Natural product | IKK activity |
| Plumbagin (5-hydroxy-2-methyl-1,4- | Natural product | IKK activity |
| naphthoquinone) | | |
| Quercetin | Natural product | IKK activity |
| Rosmarinic acid | Natural product | IKK activity |
| Semecarpus anacardiu extract | Natural product | IKK activity |
| Staurosporine | Natural product | IKK activity |
| Sulforaphane and phenylisothiocyanate | Natural product | IKK activity |
| Theaflavin (black tea component) | Natural product | Activation of IKK |
| Tilianin | Natural product | Activation of IKK |
| γ-Tocotrienol | Natural product | IKK activity |
| Wedelolactone | Natural product | IKK activity |
| Withanolides | Natural product | Activation of IKK |
| Zerumbone | Natural product | Activation of IKK |
| Silibinin | Natural product | IKKα activity; nuclear translocation |
| Betulinic acid | Natural product | IKKα activity and p65 phosphorylation |
| Ursolic acid | Natural product | IKKα activity and p65 phosphorylation |
| Monochloramine and glycine chloramine | Natural product | Oxidizes IκB |
| (NH2Cl) | | |
| Anethole | Natural product | Phosphorylation |
| Baoganning | Natural product | Phosphorylation |
| Black raspberry extracts (cyanidin 3-O- | Natural product | Phosphorylation |
| glucoside, cyanidin 3-O-(2(G)- | | |
| xylosylrutinoside), cyanidin 3-O- | | |
| rutinoside) | | |
| Buddlejasaponin IV | Natural product | Phosphorylation |
| Cacospongionolide B | Natural product | Phosphorylation |
| Calagualine | Natural product | Phosphorylation |
| Carbon monoxide | Natural product | Phosphorylation |
| Cardamonin | Natural product | Phosphorylation |
| Cycloepoxydon; 1-hydroxy-2- | Natural product | Phosphorylation |
| hydroxymethyl-3-pent-1-enylbenzene | | |
| Decursin | Natural product | Phosphorylation |
| Dexanabinol | Natural product | Phosphorylation |
| Digitoxin | Natural product | Phosphorylation |
| Diterpenes | Natural product | Phosphorylation |
| Docosahexaenoic acid | Natural product | Phosphorylation |
| Extensively oxidized low density | Natural product | Phosphorylation |
| lipoprotein (ox-LDL), 4-Hydroxynonenal | | |
| (HNE) | | |
| Flavopiridol | Natural product | IKK activity and RelA phosphorylation |
| [6]-gingerol; casparol | Natural product | Phosphorylation |
| Glossogyne tenuifolia | Natural product | Phosphorylation |
| Guggulsterone | Natural product | Phosphorylation |
| Indirubin-3′-oxime | Natural product | Phosphorylation |
| Licorce extracts | Natural product | Phosphorylation |
| Oleandrin | Natural product | Phosphorylation |
| Omega 3 fatty acids | Natural product | Phosphorylation |
| Panduratin A (from Kaempferia pandurata, | Natural product | Phosphorylation |
| Zingiberaceae) | | |
| Petrosaspongiolide M | Natural product | Phosphorylation |
| Pinosylvin | Natural product | Phosphorylation |
| Plagius flosculosus extract polyacetylene | Natural product | Phosphorylation |
| spiroketal | | |
| Phytic acid (inositol hexakisphosphate) | Natural product | Phosphorylation |
| Pomegranate fruit extract | Natural product | Phosphorylation |
| Prostaglandin A1 | Natural product | Phosphorylation/IKK |
| 20(S)-Protopanaxatriol (ginsenoside | Natural product | Phosphorylation |
| metabolite) | | |
| Rengyolone | Natural product | Phosphorylation |
| Rottlerin | Natural product | Phosphorylation |
| Saikosaponin-d | Natural product | Phosphorylation |
| Saline (low Na+ istonic) | Natural product | Phosphorylation |
| Salvia miltiorrhizae water-soluble extract | Natural product | Phosphorylation |
| Sanguinarine (pseudochelerythrine, 13- | Natural product | Phosphorylation |
| methyl-[1,3]-benzodioxolo-[5,6-c]-1,3- | | |
| dioxolo-4,5 phenanthridinium) | | |
| Sesquiterpene lactones (parthenolide; | Natural product | IKK and DNA binding |
| ergolide; guaianolides) | | |
| Scoparone | Natural product | Phosphorylation |
| Silymarin | Natural product | Phosphorylation |
| Sulindac | Natural product | IKK/Phosphorylation |
| Vesnarinone | Natural product | Phosphorylation |
| Xanthoangelol D | Natural product | Phosphorylation |
| IKKβ peptide to NEMO binding domain | Peptide | IKK-NEMO interaction |
| NEMO CC2-LZ peptide | Peptide | NEMO oligomerization |
| Adenovirus E3-14.7K | Protein, viral | IKK activity |
| Adenovirus E3-10.4/14.5K | Protein, viral | IKK activity |
| Core protein (Hepatitis C virus) | Protein, viral | IKKβ activity |
| E7 (Papillomavirus) | Protein, viral | IKK activity |
| MC160 (Mollusum Contagiosum virus) | Protein, viral | IKKα activity |
| MC159 (Mollusum contagiosum virus) | Protein, viral | Reduced IKKα expression |
| NS5B (Hepatitis C virus) | Protein, viral | IKKβ activity |
| vIRF3 (KSHV) | Protein, viral | IKKβ activity |
| Cytomegalovirus | Protein, viral | Phosphorylation |
| HB-EGF (Heparin-binding epidermal | Protein | IKK activity |
| growth factor-like growth factor) | | |
| Hepatocyte growth factor | Protein | IKK activity |
| PAN1 (aka NALP2 or PYPAF2) | Protein | IKK activity |
| PTEN (tumor suppressor) | Protein | Activation of IKK |
| Interleukin-10 | Protein | Reduced IKKα and IKKβ expression |
| Anti-thrombin III | Protein | Phosphorylation |
| Chorionic gonadotropin | Protein | Phosphorylation |
| FHIT (Fragile histidine triad protein) | Protein | Phosphorylation |
| Interferon-α | Protein | Phosphorylation |
| SOCS1 | Protein | Phosphorylation |
| AGRO100 (G-quadruplex | Synthetic | NEMO binding |
| oligodeoxynucleotide) | | |
| 2-amino-3-cyano-4-aryl-6-(2-hydroxy- | Synthetic | IKKβ activity |
| phenyl)pyridine derivatives | | |
| Acrolein | Synthetic | IKKβ activity |
| AS602868 | Synthetic | IKKβ activity |
| Aspirin, sodium salicylate | Synthetic | Phosphorylation, IKKβ |
| Dihydroxyphenylethanol | Synthetic | IKKβ activity |
| Epoxyquinone A monomer | Synthetic | IKKβ/DNA binding |
| Inhibitor 22 | Synthetic | IKKβ activity |
| MLB120 (small molecule) | Synthetic | IKKβ activity |
| Novel small-molecule inhibitor | Synthetic | IKKβ activity |
| BMS-345541 | Synthetic | IKK activity |
| CYL-19s and CYL-26z, two synthetic | Synthetic | IKK activity |
| alpha-methylene-gamma-butyrolactone | | |
| derivatives | | |
| ACHP (2-amino-6-[2- | Synthetic | IKKβ activity (ATP analog) |
| (cyclopropylmethoxy)-6-hydroxyphenyl]- | | |
| 4-piperidin-4-yl nicotinonitrile | | |
| Compound A | Synthetic | IKKβ activity (ATP analog) |
| Compound 5 | Synthetic | IKK activity |
| Cyclopentenones | Synthetic | IKKβ activity |
| Jesterone dimer | Synthetic | IKKβ activity; DNA binding |
| PS-1145 (MLN1145) | Synthetic | IKKβ activity (ATP analog) |
| 2-[(aminocarbonyl)amino]-5-acetylenyl-3- | Synthetic | IKKβ activity |
| thionphenecalboxamides | | |
| SC-514 | Synthetic | IKKβ activity |
| (Amino)imidazolylcarboxaldehyde | Synthetic | IKK activity |
| derivative | | |
| Amino-pyrimidine | Synthetic | IKK activity |
| Benzoimidazole derivative | Synthetic | IKK activity |
| CDDO-Me (synthetic triterpenoid) | Synthetic | IKK activity |
| CHS 828 (anticancer drug) | Synthetic | IKK activity |
| Diaylpyridine derivative | Synthetic | IKK activity |
| Imidazolylquinoline-carboxaldehyde | Synthetic | IKK activity |
| derivative | | |
| Indolecarboxamide | Synthetic | IKK activity |
| LF15-0195 (analog of 15- | Synthetic | IKK activity |
| deoxyspergualine) | | |
| ML120B | Synthetic | IKK activity |
| MX781 (retinoid antagonist) | Synthetic | IKK activity |
| NSAIDs | Synthetic | IKK activity |
| N-(4-hydroxyphenyl) retinamide | Synthetic | IKK activity |
| Pyrazolo[4,3-c]quinoline derivative | Synthetic | IKK activity |
| Pyridooxazinone derivative | Synthetic | IKK activity |
| Scytonemin | Synthetic | IKK activity |
| Survanta (Surfactant product) | Synthetic | IKK activity |
| Sulfasalazine | Synthetic | IKKα and IKKβ kinase activity |
| Sulfasalazine analogs | Synthetic | IKK kinase activity |
| Thalidomide | Synthetic | IKK activity |
| Azidothymidine (AZT) | Synthetic | Phosphorylation |
| BAY-11-7082 (E3((4-methylphenyl)- | Synthetic | Phosphorylation |
| sulfonyl)-2-propenenitrile) | | |
| BAY-11-7083 (E3((4-t-butylphenyl)- | Synthetic | Phosphorylation |
| sulfonyl)-2-propenenitrile) | | |
| Benzyl isothiocyanate | Synthetic | Phosphorylation |
| Carboplatin | Synthetic | Phosphorylation |
| Gabexate mesylate | Synthetic | Phosphorylation |
| Gleevec (Imatanib) | Synthetic | Phosphorylation |
| Hydroquinone | Synthetic | Phosphorylation |
| Ibuprofen | Synthetic | Phosphorylation |
| Inhaled isobutyl nitrite | Synthetic | Phosphorylation |
| Methotrexate | Synthetic | Phosphorylation |
| Monochloramine | Synthetic | Phosphorylation |
| Nafamostat mesylate | Synthetic | Phosphorylation |
| Statins (several) | Synthetic | Phosphorylation |
| THI 52 (1-naphthylethyl-6,7-dihydroxy- | Synthetic | Phosphorylation |
| 1,2,3,4-tetrahydroisoquinoline) | | |
| tetrahydroisoquinoline) | Synthetic | Phosphorylation |
| 1,2,4-thiadiazolidine derivatives | Synthetic | Phosphorylation |
| YC-1 | Synthetic | Phosphorylation |
| Mild hypothermia | — | IKK activity |
| Zinc | Inorganic complex | Degradation |
| Alachlor | Natural product | Degradation |
| Amentoflavone | Natural product | Degradation |
| Artemisia capillaris Thunb extract | Natural product | Degradation |
| Artemisia iwayomogi extract | Natural product | Degradation |
| L-ascothic acid | Natural product | Degradation |
| Antrodia camphorata | Natural product | Degradation |
| Aucubin | Natural product | Degradation |
| Baicalein | Natural product | Degradation |
| β-lapachone | Natural product | Degradation |
| Blackberry extract | Natural product | Degradation |
| Buchang-tang | Natural product | Degradation |
| Capsaicin (8-methyl-N-vanillyl-6- | Natural product | Degradation |
| nonenamide) | | |
| Catalposide | Natural product | Degradation |
| Cyclolinteinone (sponge sesterterpene) | Natural product | Degradation |
| Dihydroarteanniun | Natural product | Degradation |
| Docosahexaenoic acid | Natural product | Degradation |
| Emodin (3-methyl-1,6,8- | Natural product | Degradation |
| trihydroxyanthraquinone) | | |
| Ephedrae herba (Mao) | Natural product | Degradation |
| Equol | Natural product | Degradation |
| Erbstatin (tyrosine kinase inhibitor) | Natural product | Degradation |
| Estrogen (E2) | Natural product | Degradation/and various other steps |
| Ethacrynic acid | Natural product | Degradation (and DNA binding) |
| Fosfomycin | Natural product | Degradation |
| Fungal gliotoxin | Natural product | Degradation |
| Gamisanghyulyunbueum | Natural product | Degradation |
| Genistein (tyrosine kinase inhibitor) | Natural product | Degradation; caspase cleavage of IκBα |
| Genipin | Natural product | Degradation |
| Glabridin | Natural product | Degradation |
| Glucosamine sulfate | Natural product | Degradation |
| Glutamine | Natural product | Degradation |
| Gumiganghwaltang | Natural product | Degradation |
| Isomallotochromanol and | Natural product | Degradation |
| isomallotochromene | | |
| Kochia scoparia fruit (methanol extract) | Natural product | Degradation |
| Leflunomide metabolite (A77 1726) | Natural product | Degradation |
| Melatonin | Natural product | Degradation |
| 5′-methylthioadenosine | Natural product | Degradation |
| Midazolam | Natural product | Degradation |
| Momordin I | Natural product | Degradation |
| Mosla dianthem extract | Natural product | Degradation |
| Morinda officinalis extract | Natural product | Degradation |
| Opuntia ficus indica va saboten extract | Natural product | Degradation |
| β-Phenylethyl (PEITC) and 8- | Natural product | Degradation |
| methylsulphinyloctyl isothiocyanates | | |
| (MSO) (watercress) | | |
| Platycodin saponins | Natural product | Degradation |
| Polymyxin B | Natural product | Degradation |
| Poncirus trifoliata fruit extract | Natural product | Degradation |
| Probiotics | Natural product | Degradation |
| Prostaglandin 15-deoxy-Δ(12,14)-PGJ(2) | Natural product | Degradation |
| Resiniferatoxin | Natural product | Degradation |
| Stinging nettle (Urtica dioica) plant | Natural product | Degradation |
| extracts | | |
| Thiopental | Natural product | Degradation |
| Tipifarnib | Natural product | Degradation |
| Titanium | Natural product | Degradation |
| TNP-470 (angiogenesis inhibitor) | Natural product | Degradation |
| Trichomomas vaginalis infection | Natural product | Degradation |
| Triglyceride-rich lipoproteins | Natural product | Degradation |
| Ursodeoxycholic acid | Natural product | Degradation |
| Xanthium strumarium L. (methanol | Natural product | Degradation |
| extract) | | |
| Penetratin | Peptide | Degradation |
| Vasoactive intestinal peptide | Peptide | Degradation (and CBP-RelA interaction) |
| K1L (Vaccinia virus protein) | Protein, viral | Degradation |
| Nef (HIV-1) | Protein, viral | Degradation |
| Vpu protein (HIV-1) | Protein, viral | TrCP ubiquitin ligase inhibitor |
| γ-glutamylcysteine synthetase | Protein | Degradation |
| Heat shock protein-70 | Protein | Degradation |
| ST2 (IL-1-like receptor secreted form) | Protein | Degradation |
| YopJ (encoded by Yersinia | Protein, bacterial | Deubiquintinase for IκBα |
| pseudotuberculosis) | | |
| Activated protein C (APC) | Protein | Degradation |
| α-melanocyte-stimulating hormone (a- | Protein | Degradation |
| MSH) | | |
| IL-13 | Protein | Degradation |
| Intravenous immunoglobulin | Protein | Degradation |
| Murr1 gene product | Protein | Degradation |
| Neurofibromatosis-2 (NF-2; merlin) | Protein | Degradation |
| protein | | |
| Pituitary adenylate cyclase-activating | Protein | Degradation |
| polypeptide (PACAP) | | |
| SAIF (Saccharomyces boulardii anti- | Protein | Degradation |
| inflammatory factor) | | |
| Acetaminophen | Synthetic | Degradation |
| 1-Bromopropane | Synthetic | Degradation |
| Diamide (tyrosine phosphatase inhibitor) | Synthetic | Degradation |
| Dobutamine | Synthetic | Degradation |
| E-73 (cycloheximide analog) | Synthetic | Degradation |
| Ecabet sodium | Synthetic | Degradation |
| Gabexate mesylate | Synthetic | Degradation |
| Glimepiride | Synthetic | Degradation |
| Hypochlorite | Synthetic | Degradation |
| Losartin | Synthetic | Degradation |
| LY294002 (P13-kinase inhibitor) [2-(4- | Synthetic | Degradation |
| morpholinyl)-8-phenylchromone] | | |
| Pervanadate (tyrosine phosphatase | Synthetic | Degradation |
| inhibitor) | | |
| Phenylarsine oxide (PAO, tyrosine | Synthetic | Degradation |
| phosphatase inhibitor) | | |
| Phenytoin | Synthetic | Degradation |
| Sabaeksan | Synthetic | Degradation |
| U0126 (MEK inhibitor) | Synthetic | Degradation |
| Ro106-9920 (small molecule) | Synthetic | IκBα ubiqutination inhibitor |
| Low level laser therapy | — | Degradation |
| Electrical stimulation of vagus nerve | — | Degradation |
| Lactacystine, β-lactone | Natural product | Proteasome |
| Cyclosporin A | Natural product | Proteasome |
| ALLnL (N-acetyl-leucinyl-leucynil- | Peptide | Proteasome |
| norleucynal, MG101) | | |
| LLM (N-acetyl-leucinyl-leucynil- | Peptide | Proteasome |
| methional) | | |
| Z-LLnV (carbobenzoxyl-leucinyl-leucynil- | Peptide | Proteasome |
| norvalinal, MG115) | | |
| Z-LLL (N-carbobenzoxyl-L-leucinyl-L- | Peptide | Proteasome |
| leucinyl-L-norleucinal, MG132) | | |
| Ubiquitin ligase inhibitors | Peptides | Proteasome |
| Boronic acid peptide | Synthetic | Proteasome |
| PS-341 (Bortezomib) | Synthetic | Proteasome |
| Salinosporamide A (1, NPI-0052) | Synthetic | Proteasome |
| FK506 (Tacrolimus) | Synthetic | Proteasome |
| Deoxyspergualin | Synthetic | Proteasome |
| Disulfiram | Synthetic | Proteasome |
| APNE (N-acetyl-DL-phenylalanine-b- | Synthetic | Protease |
| naphthylester) | | |
| BTEE (N-benzoyl L-tyrosine-ethylester) | Synthetic | Protease |
| DCIC (3,4-dichloroisocoumarin) | Synthetic | Protease |
| DFP (diisopropyl fluorophosphate) | Synthetic | Protease |
| TPCK (N-α-tosyl-L-phenylalanine | Synthetic | Protease |
| chloromethyl ketone) | | |
| TLCK (N-α-tosyl-L-lysine chloromethyl | Synthetic | Protease |
| ketone) | | |
| Antrodia camphorata extract | Natural product | IκBα upregulation |
| Apigenin (4′,5,7-trihydroxyflavone) | Natural product | IκBα upregulation |
| Glucocorticoids (dexamethasone, | Natural product | IκBα upregulation |
| prednisone, methylprednisolone) | | |
| Human breast milk | Natural product | IκBα upregulation |
| α-pinene | Natural product | IκBα upregulation |
| Agastache rugosa leaf extract | Natural product | Nuclear translocation |
| Alginic acid | Natural product | Nuclear translocation |
| Astragaloside IV | Natural product | Nuclear translocation |
| Atorvastatin | Natural product | Nuclear translocation |
| 2′,8″-biapigenin | Natural product | RelA nuclear translocation |
| Blue honeysuckle extract | Natural product | Nuclear translocation |
| Buthus martensi Karsch extract | Natural product | Nuclear translocation |
| Chiisanoside | Natural product | RelA Nuclear translocation |
| 15-deoxyspergualin | Natural product | Nuclear translocation |
| Eriocalyxin B | Natural product | Nuclear translocation |
| Gangliosides | Natural product | Nuclear translocation |
| Harpagophytum procumbens (Devil's | Natural product | Nuclear translocation |
| Claw) extracts | | |
| Hirsutenone | Natural product | Nuclear translocation |
| JM34 (benzamide derivative) | Natural product | Nuclear translocation |
| KIOM-79 (combined plant extracts) | Natural product | Nuclear translocation |
| Leptomycin B (LMB) | Natural product | Nuclear translocation |
| Nucling | Natural product | RelA nuclear translocation |
| o,o′-bismyristoyl thiamine disulfide (BMT) | Natural product | Nuclear translocation |
| Oregonin | Natural product | RelA nuclear translocation |
| 1,2,3,4,6-penta-O-galloyl-β-D-glucose | Natural product | RelA nuclear translocation |
| Platycodi radix extract | Natural product | RelA nuclear translocation |
| Phallacidin | Natural product | Nuclear translocation |
| Piperine | Natural product | Nuclear translocation |
| Pitavastatin | Natural product | Nuclear translocation |
| Probiotics | Natural product | RelA nuclear translocation |
| Rhubarb aqueous extract | Natural product | RelA nuclear translocation |
| Selenomethionine | Natural product | Nuclear translocation |
| Salvia miltiorrhoza Bunge extract | Natural product | Nuclear translocation |
| ShenQi compound recipe | Natural product | RelA nuclear translocation |
| Sophorae radix extract | Natural product | Nuclear translocation |
| Sopoongsan | Natural product | Nuclear translocation |
| Sphondin (furanocoumarin derivative from | Natural product | Nuclear translocation |
| Heracleum laciniatum) | | |
| Younggaechulgam-tang | Natural product | Nuclear translocation |
| Clarithromycin | Natural product | Nuclear NF-κB expression |
| 5F (from Pteri syeminpinnata L)) | Natural product | RelA expression |
| AT514 (serratamolide) | Natural product | RelA expression |
| oxidized 1-palmitoyl-2-arachidonoyl-sn- | Natural product | RelA expression |
| glycero-3-phosphorylcholine (OXPAPC) | | |
| Sorbus commixta cortex (methanol extract) | Natural product | RelA expression |
| Cantharidin | Natural product | NF-κB expression |
| Cornus officinalis extract | Natural product | NF-κB expression |
| Neomycin | Natural product | NF-κB expression |
| Paeoniflorin | Natural product | NF-κB expression |
| Rapamycin | Natural product | NF-κB expression |
| Sargassum hemiphyllum methanol extract | Natural product | NF-κB expression |
| Shenfu | Natural product | NF-κB expression |
| Tripterygium polyglycosides | Natural product | NF-κB expression |
| PN50 | Peptide | Nuclear translocation |
| Cell permeable NLS peptides | Peptide | Nuclear translocation |
| RelA peptides (P1 and P6) | Peptide | Nuclear translocation |
| Canine Distemper Virus | Protein, viral | Nuclear translocation |
| MNF (myxoma virus) | Protein, viral | Nuclear translocation |
| 3C protease (encephalomyocarditis virus) | Protein, viral | RelA expression |
| ZUD protein | Protein | Activation of NF-κB; binds p105/RelA |
| HSCO (hepatoma protein) | Protein | Accelerates RelA nuclear export |
| β-amyloid protein | Protein | IκBα upregulation |
| Surfactant protein A (SP-A) | Protein | IκBα upregulation |
| DQ 65-79 (aa 65-79 of the alpha helix of | Protein | IκBα upregulation and IKK inhibition |
| the alpha-chain of the class II HLA | | |
| molecule DQA03011) | | |
| C5a | Protein | IκBα upregulation |
| IL-10 | Protein | IκBα upregulation |
| IL-11 | Protein | IKKα; IKBα, IKBβ upregulation |
| IL-13 | Protein | IκBα upregulation |
| Fox1j | Protein | IκBβ upregulation |
| Glucorticoid-induced leucine zipper | Protein | Nuclear translocation |
| protein (GILZ) | | |
| Heat shock protein 72 | Protein | Nuclear translocation |
| Retinoic acid receptor-related orphan | Protein | Nuclear translocation |
| receptor-alpha | | |
| TAT-SR-IκBα; MTS-SR-IκBα; p105-SR | Protein | Nuclear translocation |
| ZAS3 protein | Protein | RelA nuclear translocation; DNA |
| | | competition |
| RASSF1A gene overexpression | Protein | RelA nuclear expression |
| Onconase (Ranpirnase) | Protein | NF-κB expression |
| R-etodolac | Synthetic | IκBα upregulation |
| BMD (N(1)-Benzyl-4-methylbenzene-1,2- | Synthetic | Nuclear translocation |
| diamine) | | |
| Carbaryl | Synthetic | Nuclear translocation |
| Indole-3-carbinol | Synthetic | Nuclear translocation |
| Dioxin | Synthetic | RelA nuclear translocation |
| Dehydroxymethylepoxyquinomicin | Synthetic | Nuclear translocation |
| (DHMEQ) | | |
| Dipyridamole | Synthetic | Nuclear translocation |
| Disulfiram | Synthetic | Nuclear translocation |
| Diltiazem | Synthetic | Nuclear translocation; induced |
| | | translocation of p50 dimers |
| Fluvastatin | Synthetic | Nuclear expression |
| JSH-23 (4-Methyl- -(3-phenyl-propyl)- | Synthetic | Nuclear translocation |
| benzene-1,2-diamine | | |
| KL-1156 (6-Hydroxy-7-methoxychroman- | Synthetic | Nuclear translocation |
| 2-carboxylic acid phenylamide) | | |
| Leflunomide | Synthetic | RelA nuclear expression |
| Levamisole | Synthetic | Nuclear translocation |
| MEB (2-(4-morpholynl) ethyl butyrate | Synthetic | Nuclear translocation |
| hydrochloride) | | |
| Rolipram | Synthetic | Nuclear translocation |
| SC236 (a selective COX-2 inhibitor) | Synthetic | Nuclear translocation |
| Triflusal | Synthetic | Nuclear expression |
| Volatile anesthetic treatment | Synthetic | Nuclear translocation |
| Moxifloxacin | Synthetic | RelA expression |
| Omapatrilat, enalapril, CGS 25462 | Synthetic | NF-κB expression |
| Estrogen enhanced transcript | mRNA | Nuclear translocation |
| Metals (chromium, cadmium, gold, lead, | Inorganic Complex | DNA binding |
| mercury, zinc, arsenic) | | |
| Actinodaphine (from Cinnamomum | Natural product | DNA binding |
| insularimontanum) | | |
| Anthocyanins (soybean) | Natural product | DNA binding |
| Arnica montana extract (sequiterpene | Natural product | DNA binding |
| lactones) | | |
| Artemisinin | Natural product | DNA binding |
| Baicalein (5,6,7-trihydroxyflavone) | Natural product | DNA binding |
| Bambara groundnut (Vignea subterranean) | Natural product | DNA binding |
| β-lapachone (1,2-naphthoquinone) | Natural product | DNA binding |
| Biliverdin | Natural product | DNA binding |
| Brazilian | Natural product | DNA binding |
| Calcitriol (1a,25-dihydroxyvitamin D3) | Natural product | DNA binding |
| Campthothecin | Natural product | DNA binding |
| Cancer bush (Sutherlandia frutescens) | Natural product | DNA binding |
| Capsiate | Natural product | DNA binding |
| Catalposide (stem bark) | Natural product | DNA binding |
| Cat's claw bark (Uncaria tomentosa; | Natural product | DNA binding |
| Rubiaceae); Maca | | |
| Cheongyeolsaseuptang | Natural product | DNA binding |
| Chitosan | Natural product | DNA binding |
| Chicory root (guaianolide 8-deoxylactucin) | Natural product | DNA binding |
| Chondrotin sulfate proteoglycan | Natural product | DNA binding |
| degradation product | | |
| Clarithromycin | Natural product | DNA binding |
| Cloricromene | Natural product | DNA binding |
| Compound K (from Panax ginseng) | Natural product | DNA binding |
| Cortex cinnamomi extract | Natural product | DNA Binding |
| Cryptotanshinone | Natural product | DNA binding |
| Cytochalasin D | Natural product | DNA binding |
| DA-9201 (from black rice) | Natural product | DNA binding |
| Danshenshu | Natural product | DNA binding |
| Diterpenoids from Isodon rubescens or | Natural product | DNA binding |
| Liverwort Jungermannia | | |
| ent-kaurane diterpenoids (Croton | Natural product | DNA binding |
| tonkinensis leaves) | | |
| Epinastine hydrochloride | Natural product | DNA binding |
| Epoxyquinol A (fungal metabolite) | Natural product | DNA binding |
| Erythromycin | Natural product | DNA binding/ |
| | | transactivation |
| Evodiamine | Natural product | DNA binding |
| Fish oil feeding | Natural product | DNA binding |
| Fomes fomentarius methanol extracts | Natural product | DNA binding |
| Fucoidan | Natural product | DNA binding |
| Gallic acid | Natural product | DNA binding |
| Ganoderma lucidum (fungal dried spores | Natural product | DNA binding |
| or fruting body) | | |
| Garcinol (fruit rind of Garcinia spp) | Natural product | DNA binding |
| Geranylgeraniol | Natural product | DNA binding |
| Ginkgolide B | Natural product | DNA binding |
| Glycyrrhizin | Natural product | DNA binding |
| Halofuginone | Natural product | DNA binding |
| Hematein (plant compound) | Natural product | DNA binding |
| Herbal compound 861 | Natural product | DNA binding |
| Hydroxyethyl starch | Natural product | DNA binding |
| Hydroxyethylpuerarin | Natural product | DNA binding |
| Hypericin | Natural product | DNA binding |
| Kamebakaurin | Natural product | DNA binding |
| Linoleic acid | Natural product | DNA binding |
| Lithospermi radix | Natural product | DNA binding |
| Macrolide antibiotics | Natural product | DNA binding |
| Mediterranean plant extracts | Natural product | DNA binding |
| 2-methoxyestradiol | Natural product | DNA binding; transactivation |
| 6-(Methylsulfinyl)hexyl isothiocyanate | Natural product | DNA binding; transactivation |
| (Wasabi) | | |
| Nicotine | Natural product | DNA binding |
| Ochna macrocalyx bark extracts | Natural product | DNA binding |
| Oridonin (diterpenoid from Rabdosia | Natural product | DNA binding |
| rubescens) | | |
| PC-SPES (8 herb mixture) | Natural product | DNA binding |
| 1,2,3,4,6-penta-O-galloyl-β-D-glucose | Natural product | DNA binding |
| Pepluanone | Natural product | DNA binding |
| Phyllanthus amarus extracts | Natural product | DNA binding |
| Plant compound A (a phenyl aziridine | Natural product | DNA binding and transactivation |
| precursor) | | |
| Polyozellin | Natural product | DNA binding |
| Prenylbisabolane 3 | Natural product | DNA binding |
| Prostaglandin E2 | Natural product | DNA binding and RelA nuclear |
| | | translocation |
| Protein-bound polysaccharide (PSK) | Natural product | DNA binding |
| Quinic acid | Natural product | DNA binding |
| Sanggenon C | Natural product | DNA binding |
| Sesamin (from sesame oil) | Natural product | DNA binding |
| Shen-Fu | Natural Product | DNA binding |
| Silibinin | Natural product | DNA binding |
| Sinomenine | Natural product | DNA binding |
| Sword brake fern extract | Natural product | DNA binding |
| Tanacetum larvatum extract | Natural product | DNA binding |
| Tansinones (Salvia miltiorrhiza Bunge, | Natural product | DNA binding |
| Labiatae roots) | | |
| Taurine + niacine | Natural product | DNA binding |
| Thiazolidinedione MCC-555 | Natural product | DNA binding |
| Trichostatin A | Natural product | RelA DNA binding |
| Triptolide (PG490, extract of Chinese | Natural product | DNA binding |
| herb) | | |
| Tyrphostin AG-126 | Natural product | DNA binding |
| Ursolic acid | Natural product | DNA binding |
| Withaferin A | Natural product | DNA binding |
| Xanthohumol (a hops prenylflavonoid) | Natural product | DNA binding |
| Xylitol | Natural product | DNA binding |
| Yan-gan-wan | Natural product | DNA binding |
| Yin-Chen-Hao | Natural product | DNA binding |
| Yucca schidigera extract | Natural product | DNA binding |
| Ghrelin | Peptide | DNA binding |
| Peptide YY | Peptide | DNA binding |
| Rapamycin | Peptide | DNA binding |
| A238L IκB-like protein (African Swine | Protein, viral | DNA binding |
| Fever virus) | | |
| C + V proteins (Sendai virus) | Protein, viral | DNA binding |
| E1B (Adenovirus) | Protein, viral | DNA binding |
| ICP27 (Herpes simplex virus-1) | Protein, viral | DNA binding |
| H4/N5 (Microplitis demolitor bracovirus) | Protein, viral | DNA binding |
| N53/4A (Hepatitis C) | Protein, viral | DNA binding |
| Adiponectin | Protein | DNA binding |
| AIM2 (Absent in melanoma protein) | Protein | DNA binding |
| overexpression | | |
| Angiopoietin-1 | Protein | DNA binding |
| Antithrombin | Protein | RelA-p300 interaction |
| AvrA protein (Salmonella) | Protein | DNA binding |
| β-catenin | Protein | DNA binding |
| Bromelain | Protein | DNA binding |
| Calcium/calmodulin-dependent kinase | Protein | DNA binding |
| kinase (CaMKK) (and increased | | |
| intracellular calcium by ionomycin, UTP | | |
| and thapsigargin) | | |
| CD43 overexpression | Protein | DNA binding (RelA) |
| FLN29 overexpression | Protein | DNA binding |
| FLICE-Like Inhibitory Protein (FLIP) | Protein | DNA binding |
| G-120 (Ulmus davidiana Nakai | Protein | DNA binding; IκB increases |
| glycoprotein) | | |
| Gax (homeobox protein) | Protein | DNA binding |
| HIV-1 Resistance Factor | Protein | DNA binding |
| Insulin-like growth factor binding protein-3 | Protein | DNA binding |
| Interleukin 4 (IL-4) | Protein | DNA binding |
| Leucine-rich effector proteins of | Protein | DNA binding |
| Salmonella & Shigella (SspH1 and | | |
| IpaH9.8) | | |
| NDPP1 (CARD protein) | Protein | DNA binding |
| Overexpressed ZIP1 | Protein | DNA binding |
| p8 | Protein | DNA binding |
| p202a (nterferon inducible protein) | Protein | DNA binding by p65 and p50/p65; |
| | | increases p50 |
| p21 (recombinant) | Protein | DNA binding |
| PIAS1 (protein inhibitor of activatated | Protein | RelA DNA binding |
| STAT1) | | |
| Pro-opiomelanocortin | Protein | DNA binding |
| PYPAF1 protein | Protein | DNA binding |
| Raf Kinase Inhibitor Protein (RKIP) | Protein | DNA binding |
| Rhus verniciflua Stokes fruits 36 kDa | Protein | DNA binding |
| glycoprotein | | |
| Secretory leucoprotease inhibitor (SLPI) | Protein | DNA binding |
| Siah2 | Protein | DNA binding |
| SIRT1 Deacetylase overexpression | Protein | DNA binding |
| Siva-1 | Protein | DNA binding |
| Solana nigrum L. | Protein | DNA binding |
| Surfactant protein A | Protein | DNA binding |
| Tom1 (target of Myb-1) overexpression | Protein | DNA binding |
| Transdominant p50 | Protein | DNA binding |
| Uteroglobin | Protein | DNA binding |
| Vascular endothelial growth factor (VEGF) | Protein | DNA binding |
| ADP ribosylation inhibitors (nicotinamide, | Synthetic | DNA binding |
| 3-aminobenzamide) | | |
| 7-amino-4-methylcoumarin | Synthetic | DNA binding |
| Amrinone | Synthetic | DNA binding |
| Atrovastat (HMG-CoA reductase inhibitor) | Synthetic | DNA binding |
| Benfotiamine (thiamine derivative) | Synthetic | DNA binding |
| Bisphenol A | Synthetic | DNA binding |
| Caprofen | Synthetic | DNA binding |
| Carbocisteine | Synthetic | DNA binding |
| Celecoxib and germcitabine | Synthetic | DNA binding |
| Cinnamaldehyde, 2- | Synthetic | DNA binding |
| methoxycinnamaldehyde, 2- | | |
| hydroxycinnamaldehyde | | |
| Commerical peritoneal dialysis solution | Synthetic | DNA binding |
| CP Compound (6-Hydroxy-7- | Synthetic | DNA binding |
| methoxychroman-2-carboxylic acid | | |
| phenylamide) | | |
| Cyanoguanidine CHS 828 | Synthetic | DNA binding |
| (κB site) Decoy oligonucleotides | Synthetic | DNA binding |
| Diarylheptanoid 7-(4′-hydroxy-3′- | Synthetic | DNA binding |
| methoxyphenyl)-1-phenylhept-4-en-3-one | | |
| α-difluoromethylornithine (polyamine | Synthetic | DNA binding |
| depletion) | | |
| DTD (4,10- | Synthetic | DNA binding |
| dichloropyrido[5,6:4,5]thieno[3,2-d′:3,2- | | |
| d]-1,2,3-ditriazine) | | |
| Evans Blue | Synthetic | DNA binding |
| Evodiamine | Synthetic | DNA binding |
| Fenoldopam | Synthetic | DNA binding |
| Fexofenadine hydrochloride | Synthetic | DNA binding |
| Fibrates | Synthetic | DNA binding |
| FK778 | Synthetic | DNA binding |
| Flunixin meglumine | Synthetic | DNA binding |
| Flurbiprofen | Synthetic | DNA binding |
| Hydroquinone (HQ) | Synthetic | DNA binding |
| IMD-0354 | Synthetic | DNA binding |
| JSH-21 (N1-Benzyl-4-methylbenzene-1,2- | Synthetic | DNA binding |
| diamine) | | |
| KT-90 (morphine synthetic derivative) | Synthetic | DNA binding |
| Lovastatin | Synthetic | DNA binding |
| Mercaptopyrazine | Synthetic | DNA binding |
| Mevinolin, 5′-methylthioadenosine (MTA) | Synthetic | DNA binding |
| Monomethylfumarate | Synthetic | DNA binding |
| Moxifloxacin | Synthetic | DNA binding |
| Nicorandil | Synthetic | DNA binding |
| Nilvadipine | Synthetic | DNA binding |
| Nitric oxide-donating aspirin | Synthetic | DNA binding |
| Panepoxydone | Synthetic | DNA binding |
| Peptide nucleic acid-DNA decoys | Synthetic | DNA binding |
| Perindopril | Synthetic | DNA binding |
| 6(5H)-phenanthridinone and benzamide | Synthetic | DNA binding |
| Phenyl-N-tert-butylnitrone (PBN) | Synthetic | DNA binding |
| Pioglitazone (PPARgamma ligand) | Synthetic | DNA binding |
| Pirfenidone | Synthetic | DNA binding |
| Pyridine N-oxide derivatives | Synthetic | DNA binding |
| Quinadril (ACE inhibitor) | Synthetic | DNA binding |
| Raloxifene | Synthetic | RelA DNA binding |
| Raxofelast | Synthetic | DNA binding |
| Ribavirin | Synthetic | DNA binding |
| Rifamides | Synthetic | DNA binding |
| Ritonavir | Synthetic | DNA binding |
| Rosiglitazone | Synthetic | DNA binding |
| Roxithromycin | Synthetic | DNA binding |
| Santonin diacetoxy acetal derivative | Synthetic | DNA binding |
| Serotonin derivative (N-(p-coumaroyl) | Synthetic | DNA binding |
| serotonin, SC) | | |
| Simvastatin | Synthetic | DNA binding |
| SM-7368 (small molecule) | Synthetic | DNA binding |
| T-614 (a methanesulfoanilide anti-arthritis | Synthetic | DNA binding |
| inhibitor) | | |
| Sulfasalazine | Synthetic | DNA binding |
| SUN C8079 | Synthetic | DNA binding |
| Triclosan plus cetylpyridinium chloride | Synthetic | DNA binding |
| Tobacoo smoke | Synthetic | DNA binding |
| Verapamil | Synthetic | DNA binding |
| Heat (fever-like) | — | DNA binding |
| Hypercapnic acidosis | — | DNA binding |
| Hyperosmolarity | — | DNA binding |
| Hypothermia | — | DNA binding |
| Moderate alcohol intake | — | DNA binding |
| 8-acetoxy-5-hydroxyumbelliprenin | Natural product | Transactivation |
| Adenosine and cyclic AMP | Natural product | Transactivation |
| Artemisia sylvatica sesquiterpene lactones | Natural product | Transactivation (reporter assays) |
| α-zearalenol | Natural product | Transactivation |
| BSASM (plant extract mixture) | Natural product | Transactivation |
| Bifodobacteria | Natural product | Transactivation |
| (Bacteria) | | |
| Bupleuram fruticosum phenylpropanoids | Natural product | Transactivation |
| Blueberry and berry mix (Optiberry) | Natural product | Transactivation |
| 4′-demethyl-6-methoxypodophyllotoxin | Natural product | Transactivation |
| (lignan of Linum tauricum Willd. ssp. | | |
| tauricum) | | |
| Cycloprodigiosin hycrochloride | Natural product | Transactivation |
| Eckol/Dieckol (seaweed E stolonifera) | Natural product | Transactivation |
| Extract of the stem bark of Mangifem | Natural product | NF-κB mRNA expression |
| indica L. | | |
| Fructus Benincasae Recens extract | Natural product | Transactivation |
| Glucocorticoids (dexametasone, | Natural product | Transactivation and increases IκBα levels |
| prednisone, methylprednisolone) | | |
| Gypenoside XLIX (from Gynostemma | Natural product | Transactivation (PPAR-α-dependent) |
| pentaphyllum) | | |
| Kwei Ling Ko (Tortoise shell-Rhizome | Natural product | Transactivation |
| jelly) | | |
| Ligusticum chuanxiong Hort root | Natural product | Transactivation |
| Luteolin | Natural product | P65 transactivation |
| Manassantins A and B | Natural product | RelA transactivation |
| Mesuol | Natural product | RelA phosphorylation & transactivation |
| Nobiletin | Natural product | Transactivation |
| 4-phenylcoumarins (from Mania | Natural product | Transactivation |
| pluricostata) | | |
| Phomol | Natural product | Transactivation |
| Psychosine | Natural product | Transactivation |
| Qingkailing and Shuanghuanglian (Chinese | Natural product | Transactivation |
| medicinal preparations) | | |
| Saucerneol D and saucerneol E | Natural product | Transactivation |
| Smilax bockii warb extract (flavenoids) | Natural product | Transactivation |
| Trilinolein | Natural product | Transactivation |
| Uncaria tomentosum plant extract | Natural product | Transactivation |
| Witheringia solanacea leaf extracts | Natural product | Transactivation |
| Wortmannin (fungal metabolite) | Natural product | Transactivation |
| BZLF1 (EBV protein) | Protein, viral | Tmnsactivation |
| SH gene products (Paromyxovirus) | Protein, viral | Transactivation |
| NRF (NF-κB repression factor) | Protein | Transactivation |
| PIAS3 | Protein | Transactivation |
| PTX-B (pertussis toxin binding protein) | Protein | RelA phosphorylation and transactivation |
| Antithrombin | Protein | RelA-p300 interaction |
| 17-allylamino-17-demethoxygeldanamycin | Synthetic | Transactivation |
| 6-aminoquinazoline derivatives | Synthetic | Transactivation |
| Chromene derivatives | Synthetic | Transactivation |
| D609 (phosphatidylcholine-phospholipase | Synthetic | Transactivation |
| C inhibitor) | | |
| Dimethylfumarate (DMF) | Synthetic | Nuclear translocation |
| Ethyl 2-[(3-methyl-2,5-dioxo(3- | Synthetic | Transactivation |
| pyrrolinyl)) pyrimidine-5-carboxylate | | |
| pyrimidine-5-carboxylate | | |
| Histidine | Synthetic | Transactivation |
| HIV-1 protease inhibitors (nelfinavir, | Synthetic | Transactivation |
| ritonavir, or saquinavir) | | |
| Phenethylisothiocyanate | Synthetic | Transactivation |
| Pranlukast | Synthetic | Transactivation |
| RO31-8220 (PKC inhibitor) | Synthetic | Transactivation |
| SB203580 (p38 MAPK inhibitor) | Synthetic | Transactivation |
| Tetrathiomolybdate | Synthetic | Transactivation |
| Tranilast [N-(3,4- | Synthetic | Transactivation |
| dimethoxycinnamoyl)anthranilic acid] | | |
| 3,4,5-trimethoxy-4′-fluorochalcone | Synthetic | Transactivation |
| Troglitazone | Synthetic | Transactivation |
| 9-aminoacridine (9AA) derivatives | Synthetic | RelA phosphorylation and transactivation |
| (including the antimalaria drug quinacrine) | | |
| Mesalamine | Synthetic | RelA phosphorylation & transactivation |
| Low gravity | — | Transactivation |
| Aged garlic extract (allicin) | | |
| 2 -Amino-1-methyl-6-phenylimidazo[4,5- | | |
| b]pyridine (PhIP) | | |
| Anetholdithiolthione | | |
| Apocynin | | |
| Apple juice/extracts | | |
| Aretemisa p7F (5,6,3′,5′-tetramethoxy 7,4′- | | |
| hydroxyflavone) | | |
| Astaxanthin | | |
| Benidipine | | |
| bis-eugenol | | |
| Bruguiera gymnorrhiza compounds | | |
| Butylated hydroxyanisole (BHA) | | |
| Caffeic Acid Phenethyl Ester (3,4- | | |
| dihydroxycinnamic acid, CAPE) | | |
| Carnosol | | |
| β-Carotene | | |
| Carvedilol | | |
| Catechol derivatives | | |
| Celasterol | | |
| Cepharanthine | | |
| Chlorophyllin | | |
| Chlorogenic acid | | |
| Cocoa polyphenols | | |
| Curcumin (Diferulolylmethane) | | |
| Dehydroevodiamine | | |
| Dehydroepiandrosterone (DHEA) | | |
| and DHEA-sulfate (DHEAS) | | |
| Dibenzylbutyrolactone lignans | | |
| Diethyldithiocarbamate (DDC) | | |
| Diferoxamine | | |
| Dihydroisoeugenol | | |
| Dihydrolipoic acid | | |
| Dilazep + fenofibric acid | | |
| Dimethyldithiocarbamates (DMDTC) | | |
| Dimethylsulfoxide (DMSO) | | |
| Disulfiram | | |
| Ebselen | | |
| Edaravone | | |
| EPC-K1 (phosphodiester compound of | | |
| vitamin E and vitamin C) | | |
| Epigallocatechin-3-gallate (EGCG; green | | |
| tea polyphenols) | | |
| Ergothioneine | | |
| Ethyl pyruvate (glutathione depletion) | | |
| Ethylene glycol tetraacetic acid (EGTA) | | |
| Extract of the stem bark of Mangifem | | |
| indica L. | | |
| Flavonoids (Crataegus; Boerhaavia diffusa | | |
| root; xanthohumol) | | |
| Gamma-glutamylcysteine synthetase | | |
| (gamma-GCS) | | |
| Ganoderma lucidum polysaccharides | | |
| Garcinol (from extract of Garcinia indica | | |
| fruit rind) | | |
| Ginkgo biloba extract | | |
| Glutathione | | |
| Hematein | | |
| Hydroquinone | | |
| 23-hydroxyursolic acid | | |
| IRFI 042 (Vitamin E-like compound) | | |
| Iron tetrakis | | |
| Isovitexin | | |
| Kangen-karyu extract | | |
| Ketamine | | |
| L-cysteine | | |
| Lacidipine | | |
| Lazaroids | | |
| Ligonberries | | |
| α-lipoic acid | | |
| Lupeol | | |
| Magnolol | | |
| Maltol | | |
| Manganese superoxide dismutase (Mn- | | |
| SOD) | | |
| Mangiferin | | |
| Melatonin | | |
| Mulberry anthocyanins | | |
| Myricetin | | |
| Naringin | | |
| N-acetyl-L-cysteine (NAC) | | |
| Nacyselyn (NAL) | | |
| N-ethyl-maleimide (NEM) | | |
| Nitrosoglutathione | | |
| Nordihydroguaiaritic acid (NDGA) | | |
| Ochnaflavone | | |
| Orthophenanthroline | | |
| PMC (2,2,5,7,8-pentamethyl-6- | | |
| hydroxychromane) | | |
| Pentoxyifylline (1-(5′-oxohexyl) 3,7- | | |
| dimehylxanthine, PTX) | | |
| Phenolic antioxidants (Hydroquinone and | | |
| tert-butyl hydroquinone) | | |
| Phenylarsine oxide (PAO, tyrosine | | |
| phosphatase inhibitor) | | |
| Phyllanthus urinaria | | |
| Pyrithione | | |
| Pyrrolinedithiocarbamate (PDTC) | | |
| Quercetin (low concentrations) | | |
| Quinozolines | | |
| Rebamipide | | |
| Red wine | | |
| Ref-1 (redox factor 1) | | |
| Resveratrol | | |
| Rg(3), a ginseng derivative | | |
| Rotenone | | |
| Roxithromycin | | |
| S-allyl-cysteine (SAC, garlic compound) | | |
| Sauchinone | | |
| Spironolactone | | |
| Strawberry extracts | | |
| Taxifolin | | |
| Tempol | | |
| Tepoxaline (5-(4-chlorophenyl)-N- | | |
| hydroxy-(4-methoxyphenyl)-N-methyl- | | |
| 1H-pyrazole-3-propanamide) | | |
| Tetracylic A | | |
| α-tocopherol | | |
| α-torphryl acetate | | |
| α-torphryl succinate | | |
| Vitamin C | | |
| Vitamin B6 | | |
| Vitamin D | | |
| Vitamin E derivatives | | |
| Wogonin (5,7-dihydroxy-8- | | |
| methoxyflavone) | | |
| Yakuchinone A and B | | |
| Carbocysteine | Mucus Regulation | |
| CGS 25462 | Neutral | |
| | endopeptidase | |
| CHS 828 | DNA synthesis | |
| Clarithromycin | Bacterial 50S | |
| | ribosome | |
| Dipyridamole | Adenosine | |
| | deaminase/PDE | |
| Disulfiram | Acetaldehyde | |
| | dehydrogenase | |
| Fenoldopam | Dopamine receptor | |
| Fibrates | PPARα | |
| Fluvastin | HMG-CoA reductase | |
| Gleevec | BCR-ABL | |
| Leflunomide | Dehydroorotate | |
| | dehydrogenase | |
| Moxifloxacin | Bacterial DNA | |
| | Gyrase | |
| Perindopril | Angiotensin | |
| | converting enzyme | |
| Raloxifene | Estrogen Receptor | |
| Rapamycin | FK-binding protein | |
| | 12 | |
| Ritonavir | HIV-1 protease | |
| Tetrathiomolybdate | Anti-copper | |
| Triflusal | Cyclooxygenase-1 | |
| Troglitazone | PPAR-γ | |

It is contemplated that one or more of the NFkB inhibitors in the table above are specifically excluded in certain embodiments of the disclosure.

### III. ADJUVANTS

Vaccination strategies have been used for decades primarily to foster a protective immunity to protect patients from developing a disease after contact with an infectious agent. The specific immune response can be further stimulated by the coadministration of adjuvants.

Adjuvants have diverse mechanisms of action. The first mechanism of adjuvant action identified was the so-called depot effect, in which gel-type adjuvants such as aluminum hydroxide (alum) or emulsion based adjuvants such as incomplete Freund's adjuvants (IFA) associate with antigens and facilitate transport of the antigen to the draining lymph nodes, where immune responses are generated. In some embodiments, a gel-type adjuvant is used in the methods and compositions of the disclosure.

Various cytokines induced by adjuvants act on lymphocytes to promote predominantly Th1 or Th2 responses. In some embodiments, the adjuvant comprises a cytokine. Exemplary cytokines include IFN-γ, GM-CSF and interleukin-(IL)-12. It is contemplated that one or more of the adjuvants listed in this paragraph are specifically excluded in certain embodiments of the disclosure.

Another set of adjuvants act through toll-like receptors. Toll-like receptors (TLR) recognize specific patterns of microbial components, especially those from pathogens, and regulate the activation of both innate and adaptive immunity. Immature dendritic cells mature in response to these microbial components. As of yet 10 members of the TLR-family have been identified. TLR agonists can be used as adjuvants in the methods and compositions of the disclosure. TLR agonists are described further below.

TLR agonists are known in the art. TLR agonists may include an agonist to TLR1 (e.g. peptidoglycan or triacyl lipoproteins), TLR2 (e.g. lipoteichoic acid; peptidoglycan from Bacillus subtilis, E. coli 0111:B4, Escherichia coli K12, or Staphylococcus aureus; atypical lipopolysaccharide (LPS) such as Leptospirosis LPS and Porphyromonas gingivalis LPS; a synthetic diacylated lipoprotein such as FSL-1 or Pam2CSK4; lipoarabinomannan or lipomannan from M. smegmatis; triacylated lipoproteins such as Pam3CSK4; lipoproteins such as MALP-2 and MALP-404 from mycoplasma; Borrelia burgdorferi OspA; Porin from Neisseria meningitidis or Haemophilus influenza; Yersinia LcrV; lipomannan from Mycobacterium or Mycobacterium tuberculosis; Trypanosoma cruzi GPI anchor; Schistosoma mansoni lysophosphatidylserine; Leishmania major lipophosphoglycan (LPG); Plasmodium falciparum glycophosphatidylinositol (GPI); zymosan), TLR3 (e.g. double-stranded RNA, polyadenylic-polyuridylic acid (Poly(A:U)); polyinosine-polycytidylic acid (Poly(I:C)); polyinosine-polycytidylic acid high molecular weight (Poly(I:C) HMW); and polyinosine-polycytidylic acid low molecular weight (Poly(I:C) LMW)), TLR4 (e.g. LPS from Escherichia coli and Salmonella species); TLR5 (e.g. Flagellin from B. subtilis, P. aeruginosa, or S. typhimurium), TLR8 (e.g. single stranded RNAs such as ssRNA with 6UUAU repeats, RNA homopolymer (ssPolyU naked), HIV-1 LTR-derived ssRNA (ssRNA40), or ssRNA with 2 GUCCUUCAA repeats (ssRNA-DR)), TLR7 (e.g. imidazoquinoline compound imiquimod, Imiquimod VacciGrade™, Gardiquimod VacciGrade™, or Gardiquimod™; adenine analog CL264; base analog CL307; guanosine analog loxoribine; TLR7/8 (e.g. thiazoquinoline compound CL075; imidazoquinoline compound CL097, R848, or R848 VacciGrade™), TLR9 (e.g. CpG ODNs); and TLR11 (e.g. Toxoplasma gondii Profilin). In certain embodiments, the TLR agonist is a specific agonist listed above. In further embodiments, the TLR agonist is one that agonizes either one TLR or two TLRs specifically. In certain embodiments, the TLR is a TLR9 agonist listed above. It is contemplated that one or more of the adjuvants listed in this paragraph are specifically excluded in certain embodiments of the disclosure

In some embodiments, the TLR is selected from lipoteichoic acid; peptidoglycan from Bacillus subtilis, E. coli 0111:B4, Escherichia coli K12, or Staphylococcus aureus; atypical lipopolysaccharide (LPS) such as Leptospirosis LPS and Porphyromonas gingivalis LPS; a synthetic diacylated lipoprotein such as FSL-1 or Pam2CSK4; lipoarabinomannan or lipomannan from M. smegmatis; triacylated lipoproteins such as Pam3CSK4; lipoproteins such as MALP-2 and MALP-404 from mycoplasma; Borrelia burgdorferi OspA; Porin from Neisseria meningitidis or Haemophilus influenza; Yersinia LcrV; lipomannan from Mycobacterium or Mycobacterium tuberculosis; Trypanosoma cruzi GPI anchor; Schistosoma mansoni lysophosphatidylserine; Leishmania major lipophosphoglycan (LPG); Plasmodium falciparum glycophosphatidylinositol (GPI); and zymosan. It is contemplated that one or more of the adjuvants listed in this paragraph are specifically excluded in certain embodiments of the disclosure.

### IV. ANTIGENS

The term “antigen” as used herein refers to a molecule against which a subject can initiate a humoral and/or cellular immune response. Antigens can be any type of biologic molecule including, for example, simple intermediary metabolites, sugars, lipids, and hormones as well as macromolecules such as complex carbohydrates, phospholipids, nucleic acids and proteins. Common categories of antigens include, but are not limited to, viral antigens, bacterial antigens, fungal antigens, protozoa and other parasitic antigens, tumor antigens, antigens involved in autoimmune disease, allergy and graft rejection, and other miscellaneous antigens. In certain compositions and methods of the disclosure, the antigen is a peptide.

The inventors have demonstrated that the NFkB inhibitor improves the outcome of vaccines against Th1 responsive diseases and that the NFkB inhibitors work with antigens from the flavivirus family, orthomyxovirida, lentivirus, retroviruses and bacterial pathogens. The inventors have also found increased IgA antibodies in mucosal tissues, which would enable enhanced protection against sexually transmitted diseases, upper respiratory diseases and enteroviruses. Antigens useful in methods and compositions of the disclosure include, for example, antigenic components from Anthrax, Cancer, Chikungunya, Dengue (1,2,3,4-Dengue Fever), Diphtheria, E. coli, Shiga toxin-producing (STEC), Ebola, Non-Polio Enterovirus, Enterovirus D68 (EV-D68), Gonorrhea, Hepatitis A (Hep A), Hepatitis B (Hep B), Hepatitis C (Hep C), Hepatitis D (Hep D), Hepatitis E (Hep E), Herpes, Shingles, HIV, HPV, Influenza, Malaria, Measles, Viral Meningitis, Bacterial Menigitis, Mumps, Norovirus, Pertussis, Plague; Bubonic, Septicemic, Pneumonic, Pneumococcal Disease, Poliomyelitis (Polio), Pustular Rash diseases (Small pox, monkeypox, cowpox), Q-Fever, Rabies, Salmonellosis gastroenteritis (Salmonella), Severe Acute Respiratory Syndrome, Shigellosis gastroenteritis (Shigella), Smallpox, Tetanus, Tuberculosis, Varicella (Chickenpox), Viral Hemorrhagic Fever (Ebola, Lassa, Marburg), West Nile Virus, Yellow Fever, Yersenia (Yersinia), and Zika Virus Infection. It is contemplated that one or more of the antigens and antigenic components listed in this paragraph are specifically excluded in certain embodiments of the disclosure.

Further examples of antigens useful in the methods and compositions of the disclosure are provided below and throughout the disclosure.

A. Viral Antigens

Examples of viral antigens include, but are not limited to, retroviral antigens such as retroviral antigens from the human immunodeficiency virus (HIV) antigens such as gene products of the gag, pol, and env genes, the Nef protein, reverse transcriptase, and other HIV components; hepatitis viral antigens such as the S, M, and L proteins of hepatitis B virus, the pre-S antigen of hepatitis B virus, and other hepatitis, e.g., hepatitis A, B. and C, viral components such as hepatitis C viral RNA; influenza viral antigens such as hemagglutinin and neuraminidase and other influenza viral components; measles viral antigens such as the measles virus fusion protein and other measles virus components; rubella viral antigens such as proteins E1 and E2 and other rubella virus components; rotaviral antigens such as VP7sc and other rotaviral components; cytomegaloviral antigens such as envelope glycoprotein B and other cytomegaloviral antigen components; respiratory syncytial viral antigens such as the RSV fusion protein, the M2 protein and other respiratory syncytial viral antigen components; herpes simplex viral antigens such as immediate early proteins, glycoprotein D, and other herpes simplex viral antigen components; varicella zoster viral antigens such as gpI, gpII, and other varicella zoster viral antigen components; Japanese encephalitis viral antigens such as proteins E, M-E, M-E-NS 1, NS 1, NS 1-NS2A, 80% E, and other Japanese encephalitis viral antigen components; rabies viral antigens such as rabies glycoprotein, rabies nucleoprotein and other rabies viral antigen components. See Fundamental Virology, Second Edition, e's. Fields, B. N. and Knipe, D. M. (Raven Press, New York, 1991) for additional examples of viral antigens. It is contemplated that one or more of the antigens and antigenic components listed in this paragraph are specifically excluded in certain embodiments of the disclosure.

B. Bacterial Antigens

Bacterial antigens which can be used in the compositions and methods of the disclosure include, but are not limited to, pertussis bacterial antigens such as pertussis toxin, filamentous hemagglutinin, pertactin, FIM2, FIM3, adenylate cyclase and other pertussis bacterial antigen components; diptheria bacterial antigens such as diptheria toxin or toxoid and other diphtheria bacterial antigen components; tetanus bacterial antigens such as tetanus toxin or toxoid and other tetanus bacterial antigen components; streptococcal bacterial antigens such as M proteins and other streptococcal bacterial antigen components; gram-negative bacilli bacterial antigens such as lipopolysaccharides and other gram-negative bacterial antigen components; Mycobacterium tuberculosis bacterial antigens such as mycolic acid, heat shock protein 65 (HSP65), the 30 kDa major secreted protein, antigen 85A and other mycobacterial antigen components; Helicobacter pylori bacterial antigen components; pneumococcal bacterial antigens such as pneumolysin, pneumococcal capsular polysaccharides and other pneumococcal bacterial antigen components; hemophilus influenza bacterial antigens such as capsular polysaccharides and other hemophilus influenza bacterial antigen components; anthrax bacterial antigens such as anthrax protective antigen and other anthrax bacterial antigen components; rickettsiae bacterial antigens such as romps and other rickettsiae bacterial antigen component. Also included with the bacterial antigens described herein are any other bacterial, mycobacterial, mycoplasmal, rickettsial, or chlamydial antigens. It is contemplated that one or more of the antigens and antigenic components listed in this paragraph are specifically excluded in certain embodiments of the disclosure.

C. Fungal Antigens.

Fungal antigens which can be used in the compositions and methods of the disclosure include, but are not limited to, Candida fungal antigen components; histoplasma fungal antigens such as heat shock protein 60 (HSP60) and other histoplasma fungal antigen components; cryptococcal fungal antigens such as capsular polysaccharides and other cryptococcal fungal antigen components; coccidiodes fungal antigens such as spherule antigens and other coccidiodes fungal antigen components; and tinea fungal antigens such as trichophytin and other coccidiodes fungal antigen components. It is contemplated that one or more of the antigens and antigenic components listed in this paragraph are specifically excluded in certain embodiments of the disclosure.

D. Parasite Antigens

Examples of protozoa and other parasitic antigens include, but are not limited to, plasmodium falciparum antigens such as merozoite surface antigens, sporozoite surface antigens, circumsporozoite antigens, gametocyte/gamete surface antigens, blood-stage antigen pf 1 55/RESA and other plasmodial antigen components; toxoplasma antigens such as SAG-1, p30 and other toxoplasma antigen components; schistosomae antigens such as glutathione-S-transferase, paramyosin, and other schistosomal antigen components; leishmania major and other leishmaniae antigens such as gp63, lipophosphoglycan and its associated protein and other leishmanial antigen components; and trypanosoma cruzi antigens such as the 75-77 kDa antigen, the 56 kDa antigen and other trypanosomal antigen components. It is contemplated that one or more of the antigens and antigenic components listed in this paragraph are specifically excluded in certain embodiments of the disclosure.

E. Tumor Antigens

Tumor antigens which can be used in the compositions and methods of the disclosure include, but are not limited to, telomerase components; multidrug resistance proteins such as P-glycoprotein; MAGE-1, alpha fetoprotein, carcinoembryonic antigen, mutant p53, immunoglobulins of B-cell derived malignancies, fusion polypeptides expressed from genes that have been juxtaposed by chromosomal translocations, human chorionic gonadotrpin, calcitonin, tyrosinase, papillomavirus antigens, gangliosides or other carbohydrate-containing components of melanoma or other tumor cells. It is contemplated by the disclosure that antigens from any type of tumor cell can be used in the compositions and methods described herein. It is contemplated that one or more of the antigens and antigenic components listed in this paragraph are specifically excluded in certain embodiments of the disclosure.

F. Antigens Relating to Autoimmunity

Antigens involved in autoimmune diseases, allergy, and graft rejection can be used in the compositions and methods of the disclosure. For example, an antigen involved in any one or more of the following autoimmune diseases or disorders can be used in the present disclosure: diabetes mellitus, arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves opthalmopathy, sarcoidosis, primary biliary cirrhosis, uveitis posterior, and interstitial lung fibrosis. Examples of antigens involved in autoimmune disease include glutamic acid decarboxylase 65 (GAD 65), native DNA, myelin basic protein, myelin proteolipid protein, acetylcholine receptor components, thyroglobulin, and the thyroid stimulating hormone (TSH) receptor. Examples of antigens involved in allergy include pollen antigens such as Japanese cedar pollen antigens, ragweed pollen antigens, rye grass pollen antigens, animal derived antigens such as dust mite antigens and feline antigens, histocompatiblity antigens, and penicillin and other therapeutic drugs. Examples of antigens involved in graft rejection include antigenic components of the graft to be transplanted into the graft recipient such as heart, lung, liver, pancreas, kidney, and neural graft components. An antigen can also be an altered peptide ligand useful in treating an autoimmune disease. It is contemplated that one or more of the antigens and antigenic components listed in this paragraph are specifically excluded in certain embodiments of the disclosure. It is further contemplated that autoantigens are specifically excluded from embodiments of the disclosure.

Examples of miscellaneous antigens which can be used in the compositions and methods of the disclosure include endogenous hormones such as luteinizing hormone, follicular stimulating hormone, testosterone, growth hormone, prolactin, and other hormones, drugs of addiction such as cocaine and heroin, and idiotypic fragments of antigen receptors such as Fab-containing portions of an anti-leptin receptor antibody.

### V. PHARMACEUTICAL COMPOSITIONS

Administration of the compositions will typically be via any common route. This includes, but is not limited to parenteral, orthotopic, intradermal, subcutaneous, intramuscular, intraperitoneal, intranasal, or intravenous injection. In certain embodiments, a vaccine composition may be inhaled (e.g., U.S. Pat. No. 6,651,655, which is specifically incorporated by reference). Additional formulations which are suitable for other modes of administration include oral formulations. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, preferably about 25% to about 70%.

Typically, compositions are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner.

The manner of application may be varied widely. Any of the conventional methods for administration of an antibody are applicable. These are believed to include oral application on a solid physiologically acceptable base or in a physiologically acceptable dispersion, parenterally, by injection and the like. The dosage of the pharmaceutical composition will depend on the route of administration and will vary according to the size and health of the subject.

In many instances, it will be desirable to have multiple administrations of at most about or at least about 3, 4, 5, 6, 7, 8, 9, 10 or more. The administrations may range from 2 day to twelve week intervals, more usually from one to two week intervals. The course of the administrations may be followed by assays for alloreactive immune responses and T cell activity.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated.

The adjuvants, inhibitors, or antigens can be formulated for parenteral administration, e.g., formulated for injection via the intravenous, intradermal, intramuscular, sub-cutaneous, or even intraperitoneal routes. In a specific embodiment, the composition is administered by intradermal injection. In further embodiments, the composition is administered by intravenous injection. In further embodiments, the composition is administered by intramuscular injection. Compositions of the disclosure can be prepared as injectables, either as liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetable oils. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active ingredients in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

### VI. METHODS OF TREATMENT

As discussed above, the compositions and methods of using these compositions can treat a subject (e.g., prevent an infection or evoke a robust immune response to an antigen) having, suspected of having, or at risk of developing an infection or related disease.

As used herein the phrase “immune response” or its equivalent “immunological response” refers to a humoral (antibody mediated), cellular (mediated by antigen-specific T cells or their secretion products) or both humoral and cellular response directed against a protein, peptide, or polypeptide of the invention in a recipient patient. Treatment or therapy can be an active immune response induced by administration of immunogen or a passive therapy effected by administration of antibody, antibody containing material, or primed T-cells.

The presence of a cell-mediated immunological response can be determined by proliferation assays (CD4 (+) T cells) or CTL (cytotoxic T lymphocyte) assays. The relative contributions of humoral and cellular responses to the protective or therapeutic effect of an immunogen can be distinguished by separately isolating IgG and T-cells from an immunized syngeneic animal and measuring protective or therapeutic effect in a second subject. As used herein and in the claims, the terms “antibody” or “immunoglobulin” are used interchangeably.

Optionally, an antibody or preferably an immunological portion of an antibody, can be chemically conjugated to, or expressed as, a fusion protein with other proteins. For purposes of this specification and the accompanying claims, all such fused proteins are included in the definition of antibodies or an immunological portion of an antibody.

In one embodiment a method includes treatment for or prevention of a disease or condition caused by a pathogen. Furthermore, in some examples, treatment comprises administration of other agents commonly used against viral infection, such as one or more antiviral or antiretroviral compounds.

The therapeutic compositions are administered in a manner compatible with the dosage formulation, and in such amount as will be therapeutically effective. The quantity to be administered depends on the subject to be treated. Precise amounts of active ingredient required to be administered depend on the judgment of the practitioner. Suitable regimes for initial administration and boosters are also variable, but are typified by an initial administration followed by subsequent administrations.

The manner of application may be varied widely. Any of the conventional methods for administration of a polypeptide therapeutic are applicable. These are believed to include oral application on a solid physiologically acceptable base or in a physiologically acceptable dispersion, parenterally, by injection and the like. The dosage of the composition will depend on the route of administration and will vary according to the size and health of the subject.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

In certain embodiments, a subject is administered about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 445, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 micrograms, mg, μg/kg, or mg/kg (or any range derivable therein), of NFkB inhibitor, adjuvant, antigen, or composition.

A dose may be administered on an as needed basis or every 1, 2, 3, 4, 5, 6, 7, 8, 9, 10, 11, 12, 18, or 24 hours (or any range derivable therein) or 1, 2, 3, 4, 5, 6, 7, 8, 9, or times per day (or any range derivable therein). A dose may be first administered before or after signs of a condition. In some embodiments, the patient is administered a first dose of a regimen 1, 2, 3, 4, 5, 6, 7, 8, 9, 10, 11, 12 hours (or any range derivable therein) or 1, 2, 3, 4, or 5 days after the patient experiences or exhibits signs or symptoms of the condition (or any range derivable therein). The patient may be treated for 1, 2, 3, 4, 5, 6, 7, 8, 9, 10 or more days (or any range derivable therein) or until symptoms of an the condition have disappeared or been reduced or after 6, 12, 18, or 24 hours or 1, 2, 3, 4, or 5 days after symptoms of an infection have disappeared or been reduced.

### VII. COMBINATION THERAPY

The compositions and related methods, particularly administration of an adjuvant and NFkB inhibitor, may also be used in combination with the administration of traditional therapies.

In one aspect, it is contemplated that a therapy is used in conjunction with antiviral or anti-retroviral treatment. Alternatively, the therapy may precede or follow the other agent treatment by intervals ranging from minutes to weeks. In embodiments where the other agents and/or proteins or polynucleotides are administered separately, one would generally ensure that a significant period of time did not expire between the time of each delivery, such that the therapeutic composition would still be able to exert an advantageously combined effect on the subject. In such instances, it is contemplated that one may administer both modalities within about 12-24 h of each other and, more preferably, within about 6-12 h of each other. In some situations, it may be desirable to extend the time period for administration significantly, however, where several days (2, 3, 4, 5, 6 or 7) to several weeks (1, 2, 3, 4, 5, 6, 7 or 8) lapse between the respective administrations.

In yet another aspect, a vaccine may be administered as part of a prime/boost strategy. A priming vaccine dose can be administered in any of the embodiments described herein. A vaccine boost can be administered through the use of a second vaccine, either of the same type or from a different type of vaccine. Examples of such different vaccines include naked DNA vaccines or a recombinant poxvirus.

Various combinations of therapy may be employed, for example adjuvant is “A” and NFkB inhibitor is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the compositions to a patient/subject will follow general protocols for the administration of such compounds, taking into account the toxicity, if any, of the composition. It is expected that the treatment cycles would be repeated as necessary. It is also contemplated that various standard therapies, such as hydration, may be applied in combination with the

### VIII. EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Immune Potentiator for Increased Safety and Improved Protection of Vaccines by NF-kB Modulation

This example describes a method to decouple part of the inflammatory response from the antigen presenting actions of several adjuvants using an immune potentiator. Using a broad range of TLR agonists, the inventors demonstrate both in vitro and in vivo that using an immune potentiator decreases proinflammatory cytokines while maintaining adaptive immune function. In vivo, the inventors find that co-administering the immune potentiator with the 2017-2018 flu vaccine (Fluzone®) decreases side effects associated with vaccination and increases protection. Co-administration of the immune potentiator with CpG-ODN1826 (CpG) and dengue capsid protein leads to elimination of systemic proinflammatory cytokines post-vaccination and yields increased, neutralizing antibodies. Additionally, administering the immune potentiator with CpG and gp120, a HIV viral coat protein, increased serum IgG and vaginal IgA antibodies and shifted IgG antibody epitope recognition. Lastly, the inventors observed immune potentiation for several TLR agonists—implying a general approach. Immune-potentiation can be used to reduce the systemic side effects associated with inflammation for many adjuvanted vaccines (19)—creating the potential for many PRR agonists to be used safely, increasing the diversity of adaptive immune profiles and widening the scope of disease prevention and treatment.

A. Selection of Immune Potentiator

In seeking a method of immune potentiation, the inventors explored the extensive research on the TLR activation pathway. This powerful mechanistic framework let the inventors hypothesize about how TLR activation directs inflammatory cytokines and antigen presentation. As TLR pathways converge with NF-kB activation, and inflammatory and adaptive responses diverge upon which NF-kB subunit is activated, the inventors hypothesized that they could decouple these processes via selective inhibition—leading to reduced side effects but maintaining the adaptive response. Upon TLR activation, the transcription factor NF-kB primes the transcription of pro-inflammatory cytokines such as IL-6 and TNF-a, and cell surface receptors such as MHC-II, CD40, CD80 and CD86 (20-22). The NF-kB family is a family of transcription factors, consisting of two subunits: a DNA binding domain and a transcriptional activator (23,24). Each NF-kB dimer controls expression of a different set of genes for distinct cellular processes—broadly, some dimers control inflammatory expression while others control antigen presentation (23-25). Selectively modulating a pathway, the inventors conjectured, might lead to increased antigen presentation, while decreasing inflammation. NF-kB inhibitors have been widely explored for reducing cytokine expression in cancer (26-29), autoimmune disorders (30,31), and sepsis (32-34), yet they have not been explored as vaccine potentiators. This lack of experimentation may be because it is broadly understood that NF-κB activation is necessary in mounting an adequate adaptive immune response (29,35). However, only certain subunits direct antigen presentation (36). As a proof-of-concept immune potentiator the inventors chose SN50, a cell permeable peptide that consists of the nuclear localization sequence (NLS) of the NF-kB subunit, p50 which blocks the import of p50 containing dimers into the nucleus (37).

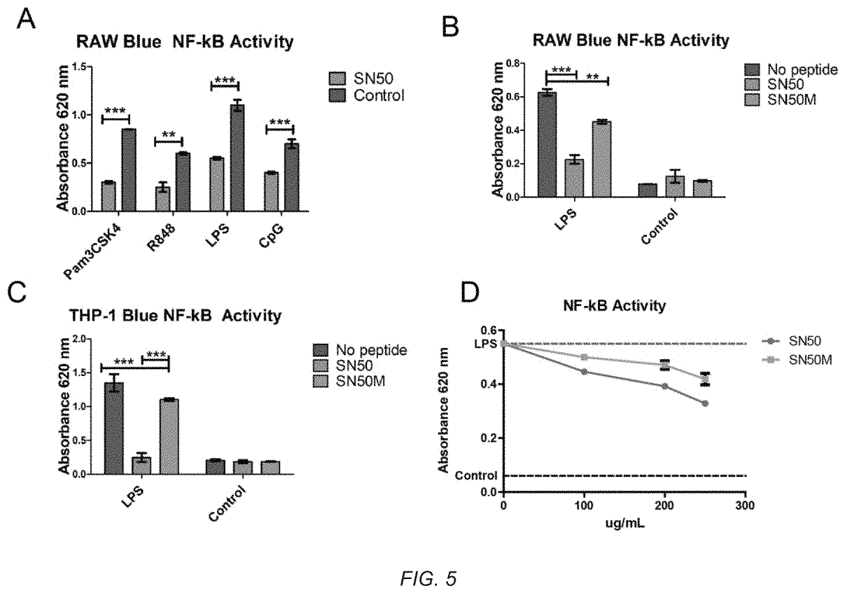

First, the inventors sought to determine if SN50 enables inhibition of NF-kB of innate immune cells. The inventors validated that SN50 reduced total NF-kB activity in human (THP-1 monocytes) and mouse (RAW macrophages) cells in a dose dependent manner. (FIG. 5A-D).

B. Examination of CpG-Induced Inflammation and Resulting Immune Response

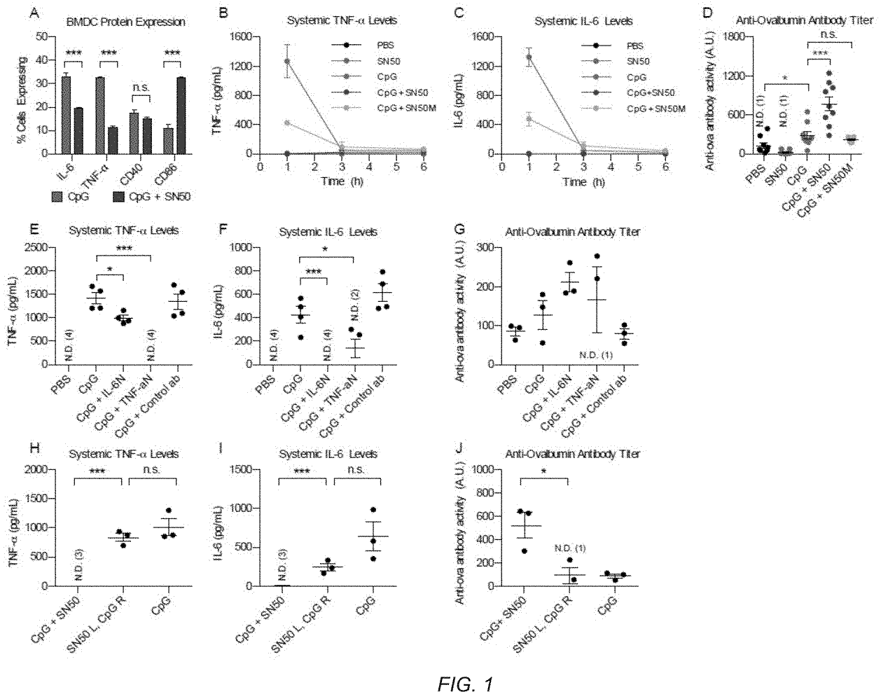

The inventors sought to verify that SN50 could enable antigen presenting cells to upregulate cell surface receptors, while limiting pro-inflammatory cytokine production. The inventors incubated murine bone marrow-derived dendritic cells (BMDCs) with SN50 and CpG or CpG alone for 6 h and analyzed how the potentiator altered cytokine production and cell surface receptor expression (FIG. 1A). Intracellular cytokine staining revealed that cells treated with SN50 demonstrated a 21% decrease in cells expressing TNF-α and a 13% decrease in cells expressing IL-6. Meanwhile, CD86 was upregulated by 22% and CD40 was only down regulated by 2.5%. Because the p65-p50 dimer is the most abundant dimer found in resting cells and involved in inflammatory cytokine production, the inventors conjecture that by inhibiting this dimer, they enable the transcription and translation of cell surface receptors while limiting inflammatory cytokines. This is consistent with previous knockout experiments (36). The result is lower inflammatory responses while priming effective adaptive immune communication.

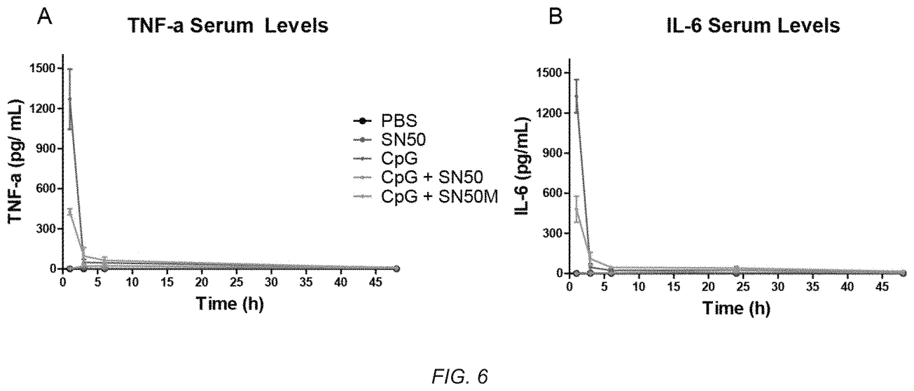

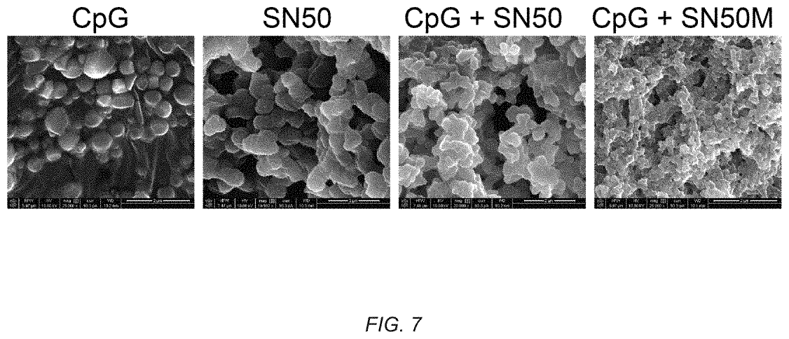

After observing that SN50 can limit inflammation without decreasing cell surface receptor expression in vitro, the inventors next wanted to examine the effect in vivo. To determine if inhibition of NF-kB could decrease the systemic levels of pro-inflammatory cytokines associated with CpG vaccination, the inventors vaccinated mice intramuscularly (i.m.) with 100 μg ovalbumin (OVA) and: PBS, SN50 (500 μg), CpG (50 μg), SN50+CpG, or SN50M (500 μg)+CpG. SN50M is a physical control for SN50 as it is a much weaker inhibitor. The inventors chose to measure systemic levels of proinflammatory cytokines TNF-α and IL-6 because high levels are unsafe and lead to side effects (17,18,38). The inventors measured these pro-inflammatory cytokines at 1 h, 3 h, 6 h, 24 h and 48 h post-injection in all groups to determine the timepoint where cytokines peak in response to CpG vaccination (FIGS. 1B, 1C, FIGS. 6A, 6B). Mice vaccinated with OVA and PBS or SN50 alone elicited no systemic cytokine response. CpG demonstrated the highest response of both TNF-α (1325 pg/mL) and IL-6 (1269 pg/mL) at the 1 h timepoint. The CpG+SN50 group showed complete elimination of cytokines for both cytokines. The CpG+SN50M group showed a decrease in cytokine levels, although not as large as observed with CpG+SN50. The inventors confirmed that this decrease in inflammatory cytokines is due to the high local inhibition of injected SN50M and not physical aggregation (FIG. 7). To determine how SN50 would affect the humoral response, the inventors analyzed serum antibody levels on day 28 (FIG. 1D). The CpG group demonstrated a 2.4-fold increase in anti-OVA antibodies compared to PBS alone. Mice vaccinated with CpG+SN50 demonstrated a 5.9-fold increase over the PBS group and 2.7-fold increase over the CpG group. These data confirmed the hypothesis that high levels of systemic TNF-α and IL-6 can be decoupled from the humoral, adaptive immune response. The inventors were surprised to find that addition of SN50 boosted the downstream adaptive response, leading to immune potentiation. Due to this increase in adaptive response and improved safety profile after vaccination, the inventors consider SN50 to be an immune potentiator.

C. Determining Mechanism of Action

To more directly examine how early systemic expression of TNF-α and IL-6 impact the immediate inflammatory response and downstream adaptive response, the inventors vaccinated mice with CpG and either TNF-a neutralizing antibody (TNF-αN) or IL-6 neutralizing antibody (IL-6N) and measured the systemic cytokines (FIGS. 1E, 1F). The CpG+IL-6N group demonstrated a 1.4-fold decrease in TNF-a expression and a complete reduction of systemic IL-6 expression. The CpG+TNF-αN group demonstrated complete elimination of systemic TNF-a and a 3-fold reduction of IL-6 expression. This result was confirmed by a control isotype antibody to rule out any nonspecific interactions. Although both IL-6N and TNF-αN groups demonstrated higher average antibody titer, these differences were not statistically significant (FIG. 1G). This indicates that reducing inflammation from CpG with the initial vaccination is not detrimental to antibody titer.

Upon observing this in vivo modulation, the inventors sought to determine whether SN50 acts locally or systemically. To examine this mechanism, the inventors injected SN50 i.m. in the left hind limb and immediately injected CpG+OVA (SN50,L+CpG,R) in the right hind limb. There was no significant difference in systemic cytokine levels tested between CpG and the SN50,L+CpG, R group (FIGS. 1H, 1I), whereas SN50+CpG injected simultaneously demonstrated reduction of TNF-α and IL-6. On day 28, the inventors analyzed antibody titer, reveling a 5.5 fold difference between the SN50+CpG and the SN50,L +CpGR group (FIG. 1J). This demonstrates the importance of coadministration of the components and therefore indicates that SN50 is acting locally to both increase safety and protection.

From these experiments, the inventors conclude that SN50 acts locally at the injection site to inhibit immediate cytokine production, containing inflammation before it is distributed systemically. Based on the inventors' in vitro data, the inventors believe CpG+SN50 enables TNF-α and IL-6 production locally at reduced levels. The inventors' in vitro data suggests that immune cells exposed to CpG+SN50 express higher levels of cell surface receptors important for antigen presentation and effective T cell activation. The inventors' experiments confirm that SN50 reduces systemic inflammation and increases antibody titer in vivo.

D. Immune Potentiation in In Vivo Influenza Challenge Model

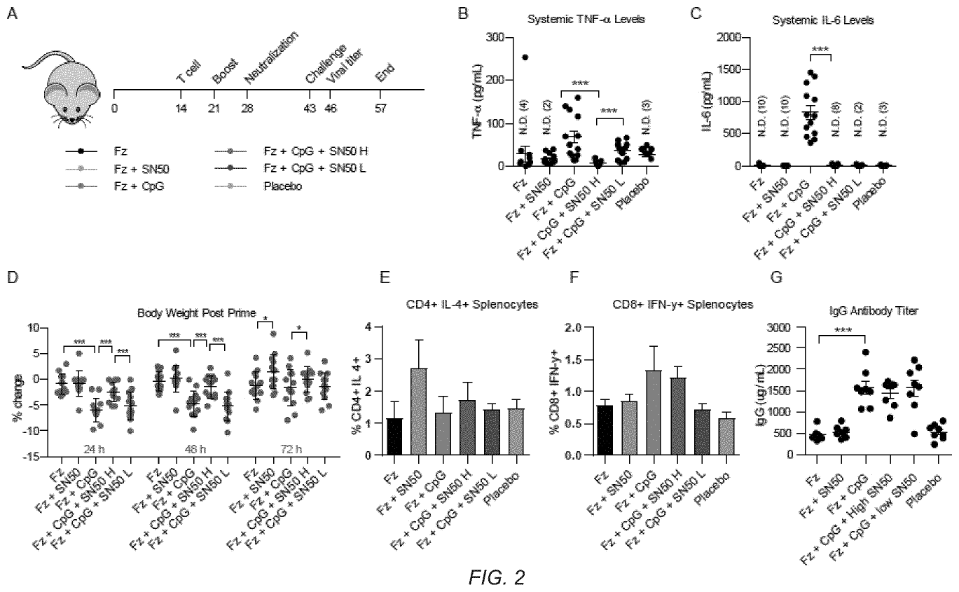

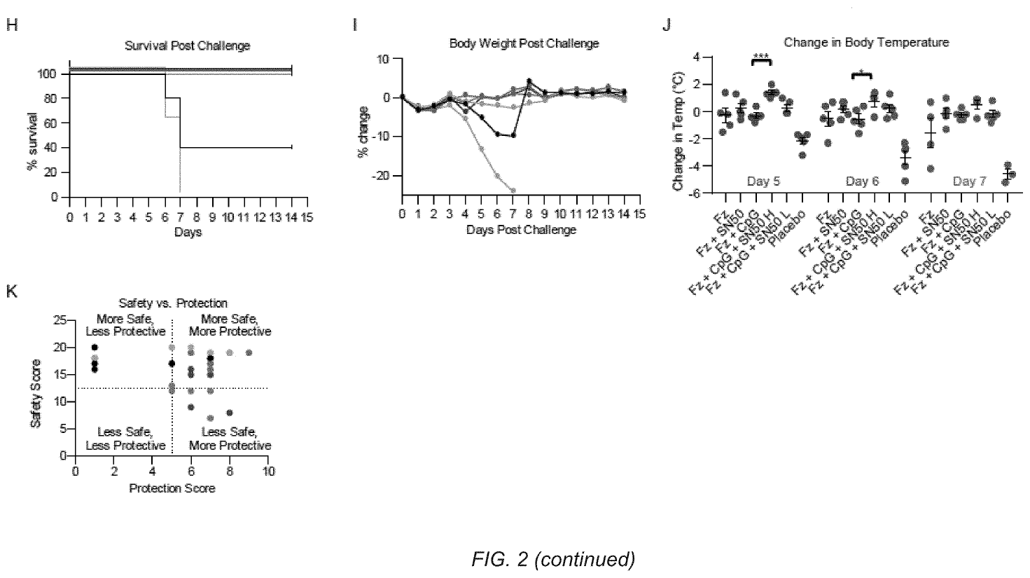

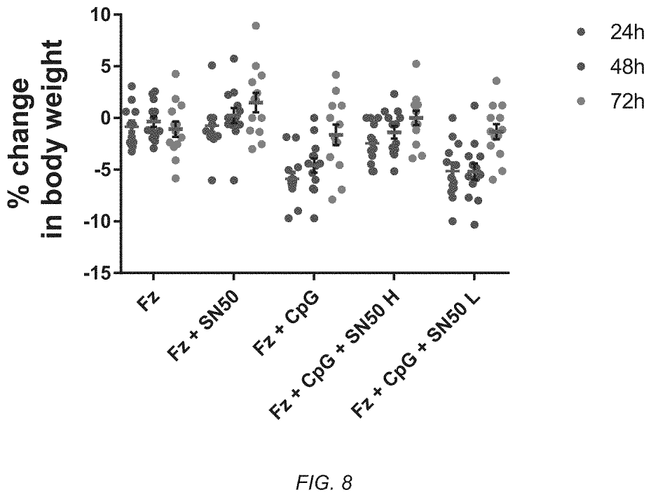

The inventors next wanted to focus on how SN50 might transition to a vaccine with challenge. The inventors selected influenza vaccine as a proof-of-concept vaccination both due to its universality and the relative ease of running animal challenges with multiple parameters. The inventors sought to determine if SN50 would reduce side effects associated with strong adjuvanticity and to see what effect this alteration on systemic cytokines would have on protection. The inventors vaccinated mice i.m. with Fluzone® quadrivalent vaccine (Fz) for the 2017/2018 influenza season, with or without CpG (50 μg) as an immune adjuvant and 500 μg SN50 (SN50 H) or 50 μg SN50 (SN50 L)) as an immune potentiator. The Fz+SN50 group demonstrated lower levels of TNF-α than Fz alone (FIGS. 2B, 2C). Across all groups, the addition of SN50 reduced levels of TNF-α and IL-6 to levels consistent with the placebo group. To examine whether SN50 can mitigate side effects from vaccination, the inventors analyzed the percent change in body weight 24, 48 and 72 h post-vaccination (FIG. 2D, FIG. 8). Weight loss is the easiest and most objective measure of side effects in mice. Mice vaccinated with Fz and Fz+SN50 lost an average of 0.85% and 0.75%, respectively by the 24 h timepoint. The Fz+CpG group lost an average of 5.9%. Adding SN50 H decreased the amount of weight loss to 2.4% and SN50 L to 5.1%. At 72 h the Fz group were −1.1% of the starting weight whereas mice vaccinated with Fz+SN50 gained +1.5%. The Fz+CpG group lost −1.6% of the starting weight and adding SN50 H lead to a reduction in weight loss (0% change) and adding SN50 L lead to −1.3% change. Overall, mice with SN50 lost less weight than mice without SN50, demonstrating that SN50 lowered side effects associated with vaccination.

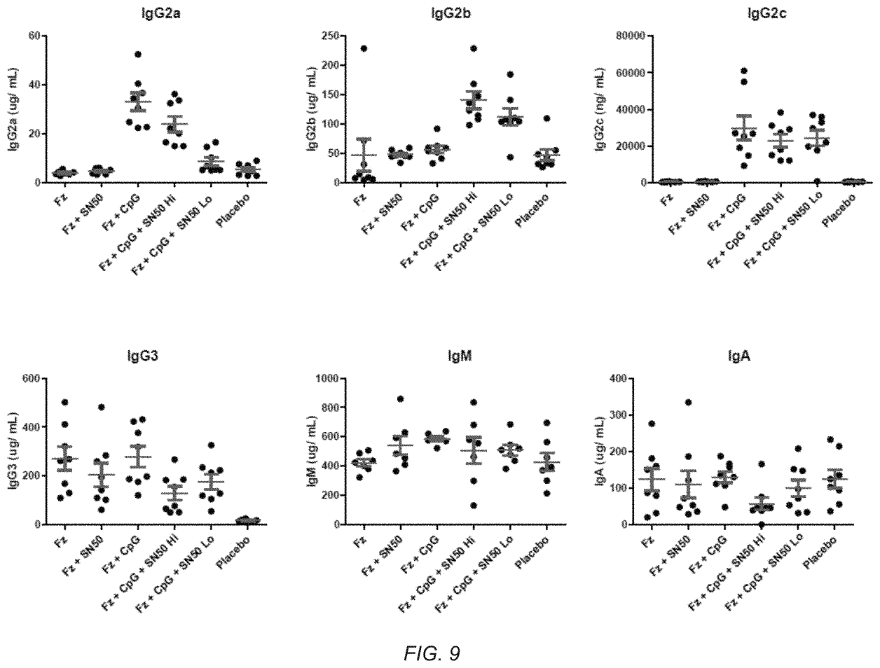

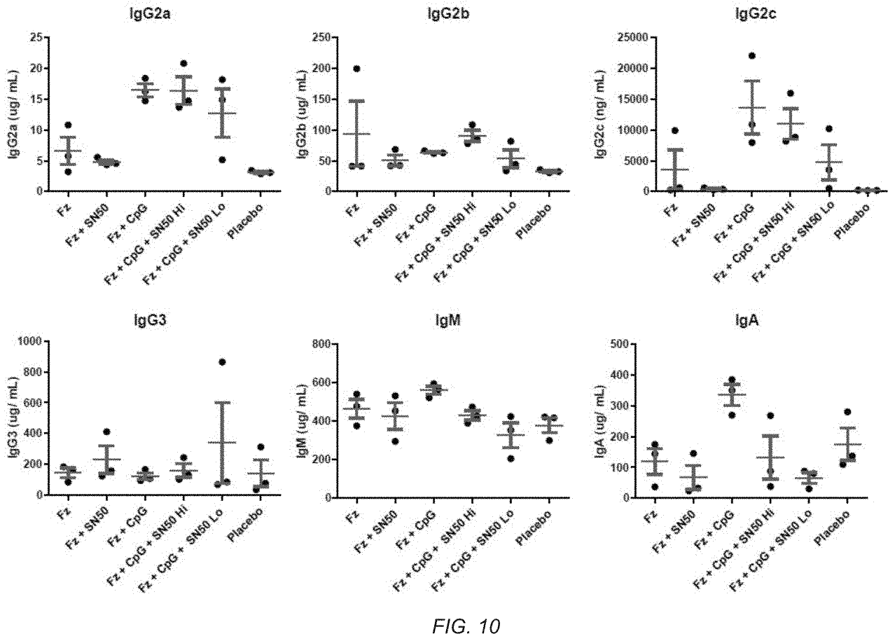

The inventors next wanted to see if the addition of SN50 would change the T cell responses or antibody titer. On day 14, the inventors analyzed splenocytes for antigen specific CD4+ and CD8+ T cells. The inventors observed no statistically significant differences between samples with and without SN50 (FIGS. 2E, 2F). On day 28, the inventors analyzed the serum for antibody levels in the blood (FIG. 2G and FIG. 9-11). There was no significant difference in IgG titer between Fz and Fz+SN50. There was a significant difference between Fz samples and Fz+CpG of 2.9 fold. There was no significant difference between groups vaccinated with CpG, implying that the addition of SN50 reduces inflammation and side effects from vaccination, while maintaining the antibody titer.

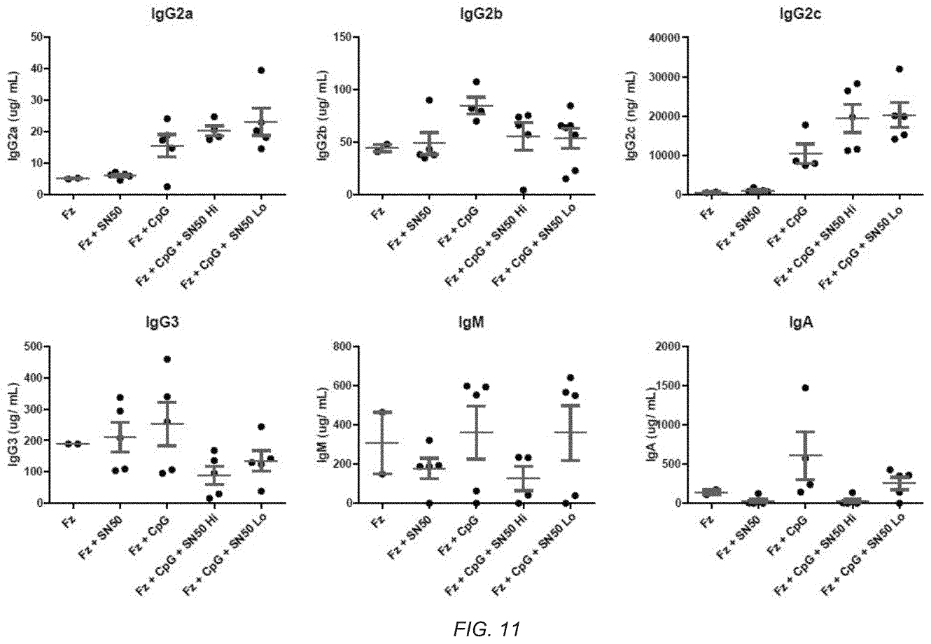

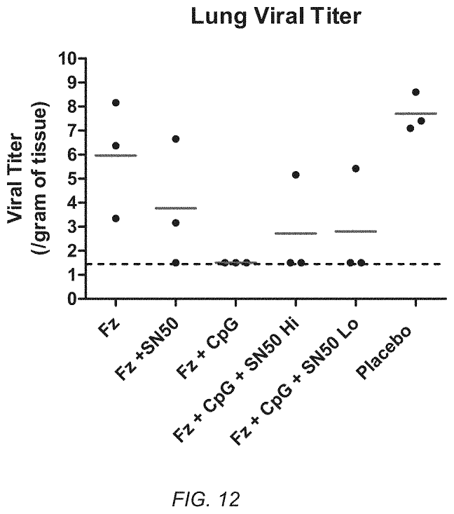

The inventors next sought to determine if SN50 would increase the protection of Fluzone. Mice were lethally challenged intranasally with 105 PFU A/Michigan/45/2015. On day 3 post-challenge the inventors analyzed the lungs of three mice for viral titer (FIG. 12). Survival was analyzed for 14 days post-challenge (FIG. 2H). By day 7, all placebo mice and 60% of the Fz mice had reached the humane endpoint and were euthanized. All other mice survived. The Fz+SN50 group was significantly more protected than the Fz alone group. The addition of SN50 to Fz+CpG confers equal protection, while improving side effects from the initial vaccination. Surprisingly, simply adding SN50 to Fz conferred enhanced protection equal to Fz+CpG group.

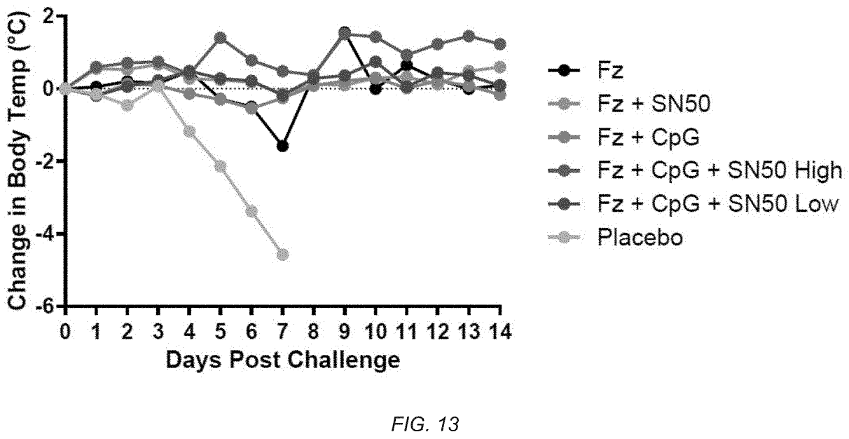

Mice were analyzed for change in body weight and body temperature for 14 days post-challenge (FIGS. 2I, 2J, FIG. 13). The peak average weight loss between Fz (−9.9%) and Fz+SN50 (−2.67%) was statistically significant. Greater weight loss is associated with a more intense infection, these data demonstrate that adding SN50 to Fz improves the response to infection. Addition of SN50 to Fz+CpG demonstrates no significant change in weight loss indicating that the SN50 can reduce systemic cytokines and side effects from vaccination with no detrimental effects to the protective response.

As an additional parameter of disease pathology, the inventors examined body temperature post-challenge. Unlike in humans, mice demonstrate a reduction in body temperature upon infection (39). The placebo has the largest peak drop in temperature (−4.57° C.), followed by the Fz group (−1.58° C.) (FIG. 2J). Adding SN50 to Fz or Fz+CpG mitigated the decrease in temperature across all groups.

Safety and protection of new vaccine adjuvants are typically considered two interdependent variables with an inverse relationship, where adequate protection is acquired by limiting safety or vice versa. As this potentiator makes the vaccine both safer and more protective, the inventors sought a single way to analyze how SN50 was changing the safety and protection profile. As these variables are considered inversely related, there are few precedents for correlation. However, a common scoring system used widely across fields is a quartile-based scoring system (40-43). Following precedent for scoring systems, the inventors developed a safety vs. protection plot (FIG. 2K). This plot is meant only to serve as a visual summary of all collected data. All groups that included SN50 in the vaccination increased both the safety and the protection of the vaccine. When all data is taken together, the inventors conclude that SN50 acts as an immune potentiator by both increasing the safety profile and improving the protective outcome of the vaccination.

Next, the inventors wanted to examine if this type of immune potentiator could improve safety and maintain the adaptive response across a broader range of diseases and antigens. The inventors chose to vaccinate against dengue and HIV because they represent additional, important diseases with active vaccine research. In each case, challenges with current methods have been identified and the inventors wanted to see if SN50 could help address those challenges, as well as maintain the current function of vaccination strategies. For dengue, the main challenge is producing antibodies that neutralize the virus, inhibiting cellular uptake. For HIV, a key challenge is in generating IgA antibodies at the mucosal interface as well as eliciting broadly neutralizing antibodies targeted to select epitopes. To explore how adding an immune potentiator affects each of these responses, the inventors analyzed each antigen set in greater detail.

E. Examination of Immune Potentiator on Dengue Neutralization

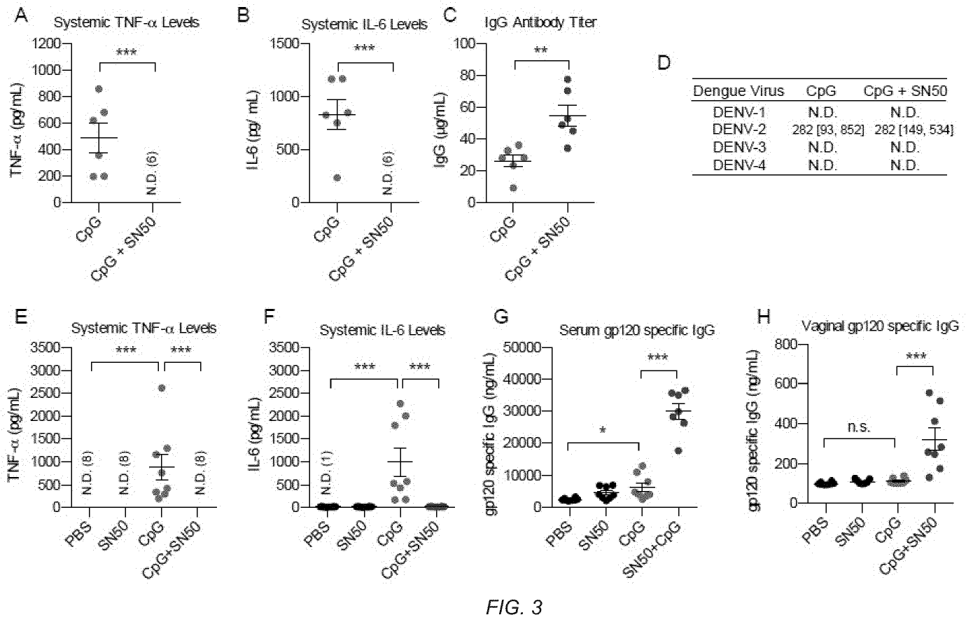

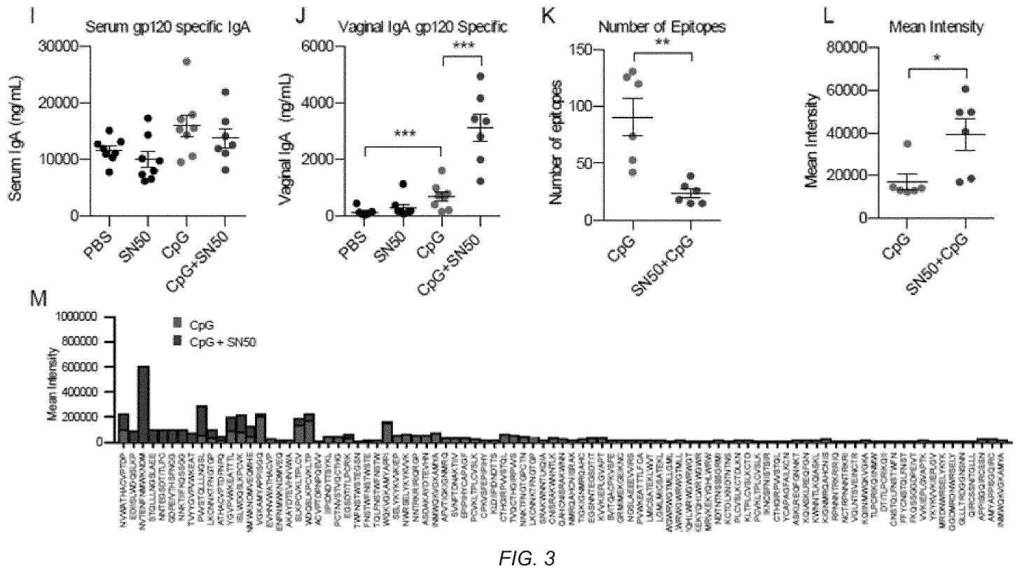

To explore dengue further, the inventors vaccinated mice with the capsid protein of dengue serotype 2 (DENV-2C) and: CpG (50 CpG+500 μg SN50. SN50 completely eliminated expression of systemic cytokines (FIGS. 3A, 3B). On day 28 the inventors analyzed the difference in antibody titer (FIG. 3C). Antibody titer in CpG+SN50 mice were almost two-fold higher than the CpG group.

To determine if SN50 alters the neutralization potential, the inventors analyzed the neutralizing titer for four strains of dengue (FIG. 3D). The inventors tested four serum samples against one strain representative of each dengue serotype. The differences in neutralization potential were not significantly different between the two groups, implying that, similar to the inventors' flu results, SN50 improves the safety while maintaining the protective responses of vaccination.

2. Analysis of Influence of Immune Potentiator on HIV Vaccination

To further test the efficacy of vaccines with SN50 and to attain a broader picture of the induced humoral immune response, the inventors vaccinated mice with gp120, a viral coat protein from HIV necessary for infection and a target of many HIV vaccines, using CpG as the immune adjuvant. Mice vaccinated with CpG demonstrated high levels of both TNF-α and IL-6, whereas all other groups including mice vaccinated with CpG+SN50 demonstrated non-detectable levels of systemic cytokines at the 1 h time point (FIGS. 3E, 3F). On day 28, the inventors analyzed antibody titers against gp120. The CpG+SN50 group induced a 4.7 fold higher anti-gp120 IgG antibody titer than the CpG group in the serum (FIGS. 3G, 3H). This demonstrates that the addition of SN50 increases IgG antibody titer across multiple antigens and suggests that it may serve as a general immune potentiator. Because mucous membranes are particularly susceptible to HIV infection, the inventors also measured the anti-gp120 IgG and IgA antibody titers in vaginal secretions (FIGS. 3I, 3J). The CpG+SN50 group demonstrated a 4.4 fold increase in anti-gp120 IgA antibodies than mice vaccinated with CpG alone. These results suggest that SN50 with gp120 may help induce class-switching to IgA antibody isotype, while also enabling localization to the mucous membranes.

The inventors next chose to determine if there were any alterations in the gp120 epitopes recognized by the resulting antibodies, using an overlapping peptide microarray. Interestingly, the number of epitopes recognized by CpG alone was higher than antibodies collected from CpG+SN50 mice; however, the fluorescent mean intensity of recognized epitopes is higher in the CpG+SN50 mice (FIGS. 3K, 3L)—implying a higher concentration of antibodies against those epitopes. Upon closer inspection of particular epitopes recognized, the inventors saw that adding the immune potentiator to the formulation shifts the epitope recognition, as different epitopes are recognized in the CpG alone and CpG+SN50, often exclusively in one condition or the other (FIG. 3M). This may prove valuable with diseases where the current recognized epitopes are not effective enough to provide protection. The most highly recognized epitope in the CpG+SN50 group corresponds to the epitope recognized by the recently isolated 35O22 monoclonal antibody (46). Antibodies isolated from mice vaccinated with CpG+SN50 also recognize the CD4 binding site recognized by several potent, broadly neutralizing antibodies (VRC 01, VRC03, b12). From these data the inventors demonstrate that the addition of SN50 shifts the epitope selectivity in the case of gp120. Based on the epitopes recognized by the serum samples, the inventors hypothesize that these antibodies may be more broadly neutralizing.

3. Improvement of Adjuvant Responses Across a Variety of TLRs and Species

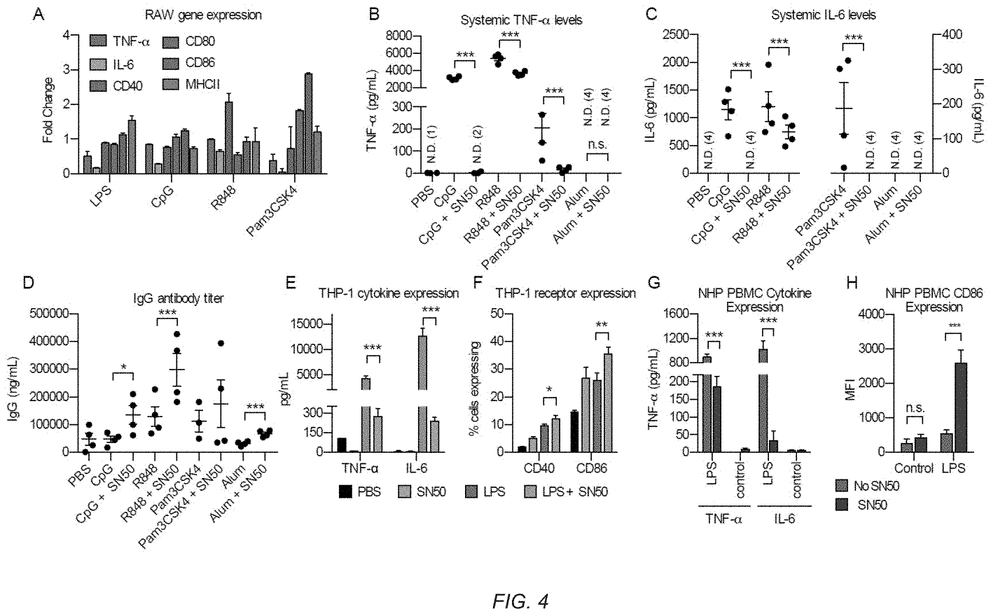

To examine the effects of the SN50 across a broad range of TLR agonists, the inventors performed qPCR on RAW macrophages treated with SN50 followed by stimulation with agonists of different TLRs. The inventors stimulated cells with SN50 and LPS (10 ng/mL), CpG (5 μg/mL), R848 (1 μg/mL) and Pam3CSK4 (100 ng/mL) and compared transcript levels to cells treated with TLR agonist alone (FIG. 4A). The inventors chose these TLR agonists because they represent a subset of the compounds with promising potential for commercial use if the inflammatory side effects can be controlled. In RAW macrophages, the inventors observed downregulation of TNF-α and IL-6 pro-inflammatory cytokine transcript levels. Across all agonists, the cell surface receptors CD86 and MHCII transcript levels were upregulated, compared to agonist alone, implying that cellular communication of the APC to the T cell may not be attenuated by the addition of SN50 and subsequent reduction in cytokine production.

To examine how this would translate in vivo, the inventors vaccinated mice with CpG (50 μg), Pam3CSK4 (20 μg) and R848 (50 μg) using gp120 as the antigen. The inventors chose to run these adjuvants alongside the most widely employed adjuvant, alum (250 μg).

With CpG, the inventors observed complete elimination of systemic TNF-α and IL-6 proinflammatory cytokines (FIGS. 4B, 4C). With R848 and Pam3CSK4 the inventors saw a significant decrease in systemic cytokines. The inventors hypothesize that SN50 is less effective at decreasing cytokines with R848 due to the low molecular weight of the molecule, enabling more rapid systemic distribution. Alum alone did not evoke a systemic cytokine response and the addition of SN50 did not alter the cytokine profile. The addition of SN50 increased the antibody titer for all adjuvants, including alum, demonstrating the broad potential use of this system to a large number of immune adjuvants (FIG. 4D).

To understand how this may translate to human vaccinations, the inventors treated THP-1 monocytes with 1/mL LPS with or without SN50. Cells treated with SN50 and LPS expressed dramatically lower levels of TNF-α and IL-6 (FIG. 4E). The inventors also observed increased levels of CD40 and CD86 (FIG. 4F). Additionally, the inventors examined the effects of SN50 on non-human primate (NHP) primary peripheral blood mononuclear cells (PBMCs). The inventors stimulated NHP PBMCs with SN50 and LPS or LPS alone for 6 h and analyzed the cell supernatant for pro-inflammatory cytokines. Cells stimulated with LPS demonstrated high levels of TNF-α and IL-6 in the cell supernatant, cells with SN50 demonstrated significant reduction in cytokine levels (FIG. 4G). The inventors observed that CD86 expression was upregulated 2-fold in cells stimulated with SN50 and LPS compared to cells stimulated with LPS alone (FIG. 4H). This implies that SN50 may work similarly in NHP and humans as it does in mice.

F. Conclusion

Using a broad range of TLR agonists, the inventors show both in vitro and in vivo that a cell permeable inhibitor of the p50 subunit of NF-kB, potentiates the immune response—reducing inflammation while increasing antibody responses. Co-administration of CpG with the immune potentiator results in significantly reduced levels of proinflammatory cytokines, often at undetectable levels. At the same time, this reduction in inflammation results in a 3-fold increase in the IgG titer of antibodies for the model antigen OVA. The inventors examined how potentiation would enhance the capabilities of the adjuvants to improve the immune response. In the inventors' influenza model the inventors directly examined side effects in response to the current commercial flu vaccine and Fz+CpG and determine that adding SN50 reduces side effects and systemic pro-inflammatory cytokine levels. The inventors also demonstrate that the safety profile can be enhanced without negatively effecting the protective response. After vaccinated mice were challenged with influenza A, mice with SN50 added to the vaccine, lead to increased survival, less weight loss and less change in body temperature. To study the effects of potentiation on TLR agonists as vaccine adjuvants, the inventors selected three diseases—influenza, dengue and HIV—all of which have had different challenges in vaccine development. In dengue vaccination, the goal is to increase antibody neutralization potential while maintaining a safe profile. The inventors demonstrate that there are no detrimental effects to dengue neutralization of antibodies with SN50, enabling us to mitigate side effects but maintain the protective response. In HIV, the inventors vaccinated with HIV envelope protein gp120, CpG and SN50, increased both IgG and IgA titers. This method appears quite general as it works with many TLR agonists and antigens. SN50 is one of hundreds of similar NF-kB inhibitors. When used in combination with the appropriate TLR agonist, many may prove useful for eliciting specific and potentially tunable responses for distinct vaccines or immunotherapies. This methodology may find use in reducing the systemic side effects associated with inflammation seen in many adjuvanted vaccines (19). This method has the potential to enable a variety of PRR agonists to be used safely in vaccines, increasing the diversity of adaptive immune profiles and widening the scope of disease prevention and treatment.

In conclusion, the inventors have demonstrated that using a specific NF-kB inhibitor in combination with common immune adjuvants can decrease pro-inflammatory cytokine production while boosting cell-surface receptor expression for effective antigen presentation and T cell activation in mouse, human and NHP primary cells. The use of this inhibitor in vivo completely reduced systemic TNF-a and IL-6 to baseline levels while increasing the downstream adaptive humoral response from the vaccination. These phenomena were observed across a broad range of antigens for a variety of pathogens demonstrating that this may prove a general strategy for improving vaccination response while conforming to strict safety standards. There are hundreds of documented immune adjuvants that provide adequate protection against diseases but induce unsafe levels of inflammation to be approved for clinical use (7,45). Additionally there are hundreds of NF-kB inhibitors, some already with FDA approval, that could be multiplexed with different TLR agonists to provide a broad range of responses (44). The inventors anticipate this framework will enable a variety of TLR agonists to be used safely in human vaccines, increasing the diversity of adaptive immune profiles and widening the scope of disease prevention and treatment.

G. Materials and Methods

1. RAW Blue NF-kB Assay

RAW-Blue™ NF-kB cells (Invivogen) were passaged and plated in a 96 well plate at 100 k cells/well in 180 μL DMEM containing 10% HIFBS. Cells were incubated at 37° C. and 5% CO₂ for 1 h. SN50 was added at indicated concentrations, cells were incubated 1 h. Immune agonists were added at their indicated concentrations. The volume of each well was brought to 200 μL and incubated at 37° C. and 5% CO₂ for 18 h. After 18 h, 20 μL of the cell supernatant was placed in 180 μL freshly prepared QuantiBlue (Invivogen) solution and incubated at 37° C. and 5% CO₂ for up to 2 h. The plate was analyzed every hour using a Multiskan FC plate reader (Thermo Scientific) and absorbance was measured at 620 nm.

2. THP-1 Blue NF-kB Assay

THP-Blue™ NF-kB cells (Invivogen) were passaged and plated in a 96 well plate at 400 k cells/well in 180 μL RPMI 1680 containing 10% HIFBS. Cells were incubated at 37° C. and 5% CO₂ for 1 h. SN50 was added at indicated concentrations and cells were incubated for 1 h. Immune agonists were added at their indicated concentrations. The volume of each well was brought to 200 μL and incubated at 37° C. and 5% CO₂ for 18 h. After 18 h, the plate was spun down at 400×g (Allegra X-30, Beckman Coulter) and 20 μL of the cell supernatant was placed in 180 μL freshly prepared QuantiBlue (Invivogen) solution and incubated at 37° C. and 5% CO₂ for up to 2 h. The plate was analyzed every hour using a Multiskan FC plate reader (Thermo Scientific) and absorbance was measured at 620 nm.

3. Gene Expression

RAW 264.7 macrophages or THP-1 cells were passaged and plated in a cell culture treated 6-well plate at 4×106 cells/well in 1.5 mL DMEM or RPMI (respectively) containing 10% HIFBS. SN50 (250 μg/mL) or PBS was added to wells and cells were incubated for 1 h at 37° C. and 5% CO₂ for 6 h. RNA was extracted using RNeasy Plus Mini kit (Qiagen). RT-PCR was performed using RT2 first strand kit (Qiagen) and BioRad thermocycler according to manufacturer's protocol. cDNA was stored at −20° C. RT2 SYBR ROX qPCR Master mix (Qiagen) was used according to manufacturer's protocol. qPCR amplification was performed using a Stratagene Mx3005P thermocycler.

4. Intracellular Cytokine Staining

a. BMDCs

Monocytes were harvested from 6-week-old C57BL/6 mice. Monocytes were differentiated into dendritic cells (BMDCs) using supplemented culture medium: RPMI 1640 (Life Technologies), 10% HIFBS (Sigma), 20 ng/mL granulocyte-macrophage colony stimulating factor (produced using “66” cell line), 2 mM Lglutamine (Life Technologies), 1% antibiotic-antimycotic (Life Technologies), and 50 μM beta-mercaptoethanol (Sigma). After 5 days of culture, BMDCs were incubated with 250 μg/mL SN50. After 1 h CpG ODN 1826 (IDT) and 1 μL/mL GolgiPlug (BD Biosciences) was added. Cells were incubated for 6 h at 37° C. in and 5% CO₂. Cells were stained for CD40 (Biologend, 124610), CD86 (Biolegend, 105109), and intracellular IL-6 (Biolegend, 504508) and TNF-a (Biolegend, 506308) cytokine production and analyzed using BD Accuri C6 flow cytometer.

b. SEM Analysis

Sample suspensions obtained directly from injection mixtures were dried for 24 h, mounted on carbon tape, and sputter coated (South Bay Technologies) with approximately 2-4 nm of Au/Pd 60:40 or Ir. Scanning electron microscopy (SEM) of the sample suspensions was performed using an FEI Quanta 3D FEG dual beam (SEM/FIB) equipped with Inca EDS (Oxford Instruments).

5. In Vivo Studies

a. Animals

All animal procedures were performed under a protocol approved by the University of Chicago Institutional Animal Care and Use Committee (IACUC). 6-8 week-old C57/B6 female mice were purchased from Jackson Laboratory (JAX). All compounds were tested for endotoxin prior to use. All vaccinations were administered intramuscularly in the hind leg. Blood was collected from the sapheneous vein at time points indicated.

Antigens were purchased from Sino Biological (HIV subgroup M, Influenza A H1N1 (A/California/04/2009) Hemagglutinin/HA Protein, Dengue virus DENV-2 (Strain New Guinea C) Capsid protein/DENV-C Protein (His Tag), Virogen (HIV-1 env (gp41) antigen) or Invitrogen (Vaccigrade Ovalbumin). Vaccigrade CpG ODN 1826 was purchased from Invivogen or Adipogen. SN50 was synthesized via solid phase peptide synthesis as previously described and purified using Gilson preparatory HPLC.

b. Vaccinations

Mice were anesthetized lightly with isoflurane and injected intramuscularly in the hind leg with 50 uL containing antigen, adjuvant and PBS. Antigen doses: ovalbumin (100 μg), DENV2-C (5 μg) and gp120 (3 ug). CpG dose, 50 μg. SN50, 500 TNF-aN, 30 μg. IL-6N, 30 μg.

C. Plasma Cytokine Analysis

Blood was collected from mice at specified time points in 0.2 mL heparin coated collection tubes (VWR Scientific). Serum was isolated via centrifugation 2000×g for 5 min. Supernatant was collected and stored at −80° C. until use. Serum was analyzed using BD Cytometric Bead Array Mouse Th1/Th2/Th17 cytokine kit or Mouse Inflammation cytokine kit according to manufacturer's protocol. Briefly, beads containing antibodies for desired cytokines were mixed with 50 μL serum and 50 μL PE detection reagent and incubated for 2 h. Beads were washed and analyzed using BD Accuri C6 flow cytometer. Data was analyzed using BD Accuri C6 software and Graphpad Prism.

D. Antibody Titer Analysis

Mice were vaccinated with indicated formulations. Blood was collected at time points indicated in 0.2 mL heparin coated collection tubes (VWR Scientific) for plasma or uncoated tubes for serum. Plasma was isolated via centrifugation (2000×g, 5 min). Serum was isolated by allowing blood to clot for 15-30 min at RT and centrifuging (2000×g for 10 min) at 4° C. Serum was analyzed using a quantitative anti-ovalbumin total Ig's ELISA kit (Alpha Diagnostic International) according to the specified protocol. Total IgG and IgA was analyzed using total mouse IgG or IgA uncoated ELISA (Invitrogen) and was analyzed using Multiskan FC plate reader (Thermo Scientific) and absorbance was measured at 450 nm. Data was analyzed using Graphpad Prism.

6. Influenza Challenge Model

a. Animals

All animal procedures were performed under a protocol approved by the Illinois Institute of Technology Research Institute (IITRI) Animal Care and Use Committee (IACUC). 6-8 week-old C57/B6 female mice were purchased from Charles River. All compounds were tested for endotoxin prior to use. All vaccinations were administered intramuscularly in the hind leg.

Initial group assignments were assigned to using a computerized randomization procedure based on body weights that produce similar group mean values [ToxData® version 3.0 (PDS Pathology Data Systems, Inc., Basel, Switzerland)]. Mice were vaccinated by i.m. injection into the hind leg on Days 0 and 21. The vaccine material used in this study is Fluzone® quadrivalent influenza vaccine (Sanofi Pasteur). Each 0.5 mL dose of Fluzone® contains at least 15 μg of hemagglutinin (HA) from each of the following four influenza strains recommended for the 2017/2018 influenza season: A/Michigan/45/2015 X-275 (H1N1)pdm09-like strain, A/Hong Kong/4801/2014 X-263B (H3N2)-like strain, B/Phuket/3073/2013-like strain and B/Brisbane/60/2008-like strain. At least 1 μg of each strain was used in vaccination of the mice.

Body weights were collected 24 hr, 48 hr and 72 hr post-prime vaccination. Body temperatures were collected 1 hr, 3 hr, 24 hr, 48 hr and 72 hr post-prime vaccination. Blood samples were collected on days 0, 14, 28, 42, 56. Plasma was collected on day 0. Serum was collected on days 14, 28, 52 and 56. Five animals from each group were humanely euthanized on day 14 post-vaccination. Spleens were collected for T cell analysis. On day 43 post-vaccination, all mice were challenged via intranasal route with a lethal dose of A/Michigan/45/2015. The dose level of challenge virus used was an equivalent of 5 LD50. For inoculation, mice were anesthetized with a ketamine (80 mg/kg) and xylazine (10 mg/kg) mixture. Once anesthetized, 0.025 mL of inoculum was delivered dropwise into the nares. The mouse was held upright to allow the virus to be inhaled thoroughly then returned to its cage. After challenge, body weights and temperature readings were recorded daily through a transponder (BioMedic data systems, Seaford, Del.) implanted subcutaneously in each mouse. Animals were monitored for morbidity/mortality for 14 days post-infection. Any animals meeting pre-determined moribund criteria (>20% weight loss) were humanely euthanized. Three animals from each group were humanely euthanized on day 3 post-challenge (Day 45) and lungs collected for viral quantitation by plaque assay/TCID50. Tissues for viral titers were weighed then flash frozen in an ethanol/dry ice bath or liquid nitrogen and stored at ≤−65° C. Frozen organs were thawed at 37° C. for 5 min. Once thawed, organs were homogenized in MEM 10% w/v using a Bead Ruptor 12 (Omni International, Kennesaw, Ga.) in tubes containing 1.4 mm ceramic beads. Homogenized organs were centrifuged at 2,000×g for 5 min to remove cellular debris. The resulting supernatant was serially diluted 10-fold then transferred into respective wells of a 96-well plate containing a monolayer of Madin-Darby Canine Kidney Cells (MDCK) cells for titration. The TCID50 assay will be performed. TCID50 titers will be calculated using the method of Reed-Meunch. The remaining 5 mice in each group were monitored for the remaining days of the challenge.

B. Neutralization Assays

Serum samples were tested against a representative of each dengue serotype (DENV-1: strain Hawaii; DENV-2 strain New Guinea C; DENV-3 strain Philippines/H87/1956 and DENV-4 strain H241). Sera was serially diluted two-fold, (starting dilution 1:100) then incubated with standardized virus concentration of 50-120 PFU of each strain. The serum:virus mixture was transferred into respective wells of a 96-well plate which contained a monolayer of Vero cells. The cells were incubated for 40 hours at 37° C. After 40 hours of incubation, the cells were fixed with 1.0% paraformaldehyde and stained by Anti-Flavivirus Group Antigen Antibody, clone D1-4G2-4-15 (Millipore Billerica, Mass.) followed by peroxidase-conjugated goat anti-mouse IgG (Kirkegaard and Perry Laboratories, Gaithersburg, Md.). Spots were developed using TrueBlue Peroxidase Substrate (Kirkegaard and Perry Laboratories, Gaithersburg, Md.). Plaques were visualized and counted using an ELISPOT instrument. Plaque reduction neutralization test titers (PRNT) were expressed in terms of conventional 50% PRNT end-point titers.

C. T Cell Analysis

Spleens were harvested from mice as described above at time point indicated. Splenocytes were isolated by pressing spleen fragments through a strainer attached to a 50-mL conical tube using a syringe plunger. Cells were washed through the strainer with PBS and centrifuged at 500×g for 10 min. Supernatant was aspirated and the pellet was resuspended in 2 mL of pre-warmed lysing solution (BD Pharm Lyse™ lysing solution) and incubated at 37° C. for 2 minutes. 30 mL of PBS was added and cell suspension was centrifuged at 500×g for 10 minutes. Supernatant was discarded and cells were resuspended in RPMI containing 10% HIFBS at 2×106 cells/mL. 500 μL was added to 24 well plate. Cells were incubated with 10 μg/mL Influenza A H1N1 (A/Michigan/45/2015) Hemagglutinin/HA1 Protein (His Tag) (SinoBiological). After 1 h GolgiStop (BD Biosciences) was added and cells were incubated for 11 h at 37° C. and 5% CO₂. Cells were centrifuged 500×g for 10 min and stained for CD4/IL-4 (Biolegend o FITC anti-mouse CD4 [RM4-5], PerCP/Cy5.5 anti-mouse IL-4 [11B11]) or CD8/IFN-y (FITC anti-mouse CD8a [53-6.7], PE anti-mouse IFN-γ [XMG1.2]) using BD Cytofix/Cytoperm Fixation/Permeabilization Solution Kit according to manufacter's protocol and analyzed using a NovoCyte flow cytometer (ACEA Biosciences, Inc.).

D. Epitope Analysis

Mouse serum was collected as described above and samples were analyzed using Multiwell RepliTope™ microarray for appropriate antigen (JPT Innovative Peptide Solutions) according to manufacturer's protocol. Briefly, serum samples were diluted in 3% BSA in 1× TBS-Buffer+0.1% Tween20 (TBS-T) to a final concentration of 10 μg/mL. The microarray was fitted with an ArraySlide 24-4 chamber (JPT Innovative Peptide Solutions) to enable multi-sample analysis. 150 uL diluted serum was added to samples wells and incubated for 1 h at 30° C. Wells were washed 5× with TBS-T. 150 μL secondary antibody (1 μg/mL) was added to wells and incubated RT for 1 h. Wells were washed 5× with TBS-T and 2× with nanopure water. Arrays were imaged using a Caliber I.D. RS-G4 confocal microscope and analyzed using ImageJ.

E. Safety and Protection Score

The inventors assigned a safety score comprised of systemic TNF-a, IL-6 levels and weight loss post-vaccination. A score for each TNF-a, IL-6 and weight loss was assigned for each mouse. The safety score of a single mouse represents the summation of these individual scores. A protection score was assigned based on survival, change in body weight and change in body temperature post-challenge. Scores were determined by dividing values into quartiles, and assigned a number 0 to 4 based on the quartile. Higher values indicate an improved safety profile (lower TNF-a or IL-6, less weight loss after vaccination) or improved protection (survival, less weight loss, higher body temperature after challenge).

f. Statistics and Replicates

Data is plotted and reported in the text as the mean±s.e.m. Sample size is as indicated in biological replicates in all in vivo and in vitro experiments. The sample sizes were chosen based on preliminary experiments or literature precedent indicating that the number would be sufficient to detect significant differences in mean values should they exist. P values were calculated using a two-tailed unpaired heteroscedastic t-test.

### Example 2

### Additional NFkB Inhibitor Studies

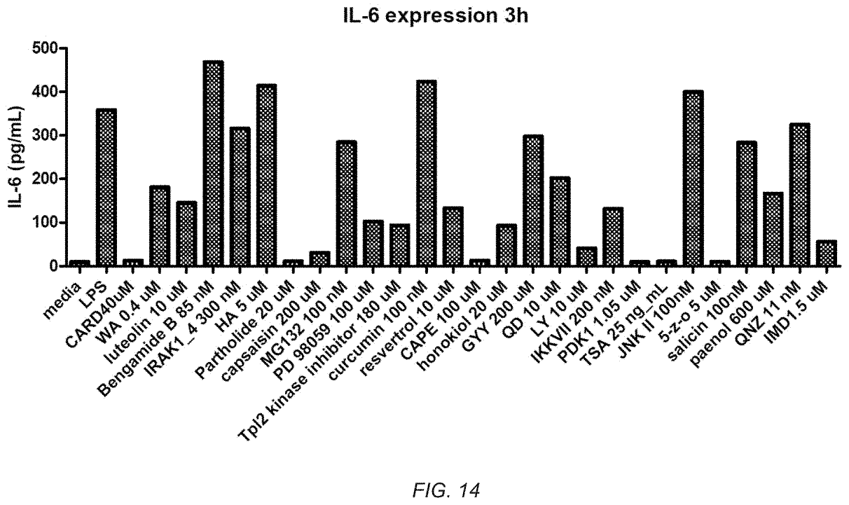

RAW macrophages were treated with NF-kB inhibitors (cardamonin, withaferin A (WA), luteolin, begamide B, IRAK 1/4 inhibitor, histone acetylase inhibitor (HA), parthenolide, capsaicin, MG132, PD 98059, Tpl2 kinase inhibitor, curcumin, resveratrol, caffeic acid phenyl ester (CAPE), honokiol, GYY, LY, IKKVII, PDK1 inhibitor, TSA, JNK II inhibitor, 5z-7-oxozeaenol (5-z-o), salicin, QNZ or IMD) and incubated for 45 min before the addition of 100 ng/mL LPS. IL-6 expression was analyzed 3 h post-activation with LPS (FIG. 14). LPS alone demonstrated high levels of IL-6 expression (362 pg/mL). Cardamonin, parthenolide, CAPE, PDK1, TSA and 5-z-o demonstrated a complete reduction of IL-6 to background levels. WA, luteolin, resveratrol, honokiol and IKKVII inhibitor demonstrated decreases in proinflammatory cytokine activity without entirely blocking expression.

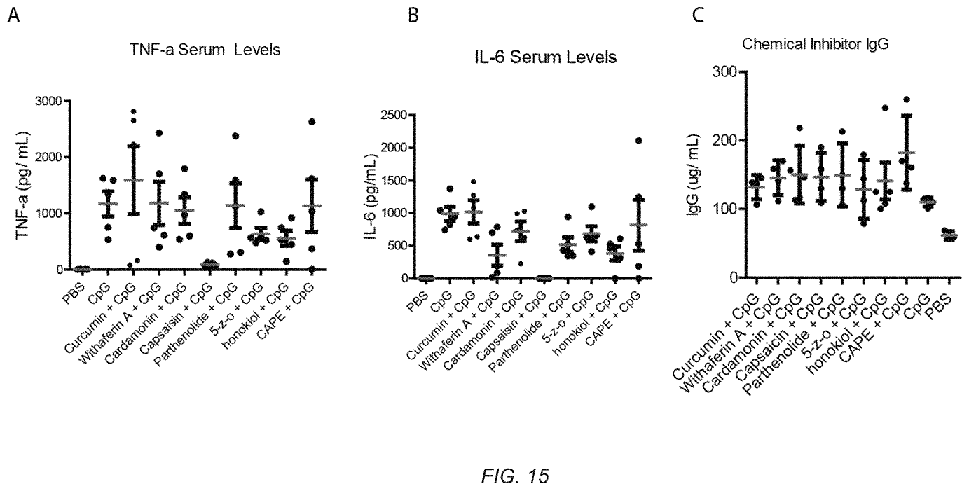

To analyze the effect further, the inventors chose to examine inhibitors from the two categories: those that completely inhibited IL-6 expression and those that decreased the expression, but not to background levels. For the inhibitors that completely inhibited IL-6 expression the inventors chose to use WA, cardamonin, parthenolide, 5-z-o and CAPE due to their bioavailability and effective dosages. For inhibitors that reduced levels of IL-6 the inventors chose to use capsaicin and honokiol. Lastly, the inventors used curcumin to examine how an inhibitor that did not demonstrate a change in IL-6 activity in vitro would have on an in vivo vaccination study. The inventors vaccinated mice with NF-kB inhibitor, CpG and ovalbumin. One hour post-vaccination, the inventors analyzed the plasma for systemic pro-inflammatory cytokines TNF-a and IL-6. CpG alone demonstrated high levels of systemic TNF-a, with the addition of curcumin increasing the average expression levels (FIG. 15A). The addition of WA, cardamonin, parthenolide and CAPE showed no change in systemic TNF-a levels compared to CpG alone. Capsaicin inhibited TNF-a expression the most returning the systemic TNF-a levels to background. Both 5-z-o and honokiol reduced the TNF-a levels by two-fold. Interestingly, the systemic IL-6 levels did not mirror the TNF-a levels (FIG. 15B). CpG alone elicited a high IL-6 response. The addition of curcumin did not significantly alter the IL-6 levels. Withaferin A, parthenolide, 5-z-o, honokiol and CAPE demonstrated about a two-fold decrease in IL-6 levels. Addition of capsaicin demonstrated IL-6 levels consistent with PBS injection. On day 21, the inventors analyzed the IgG antibody titer, all inhibitors increased the antibody titer compared to CpG alone (FIG. 15C).

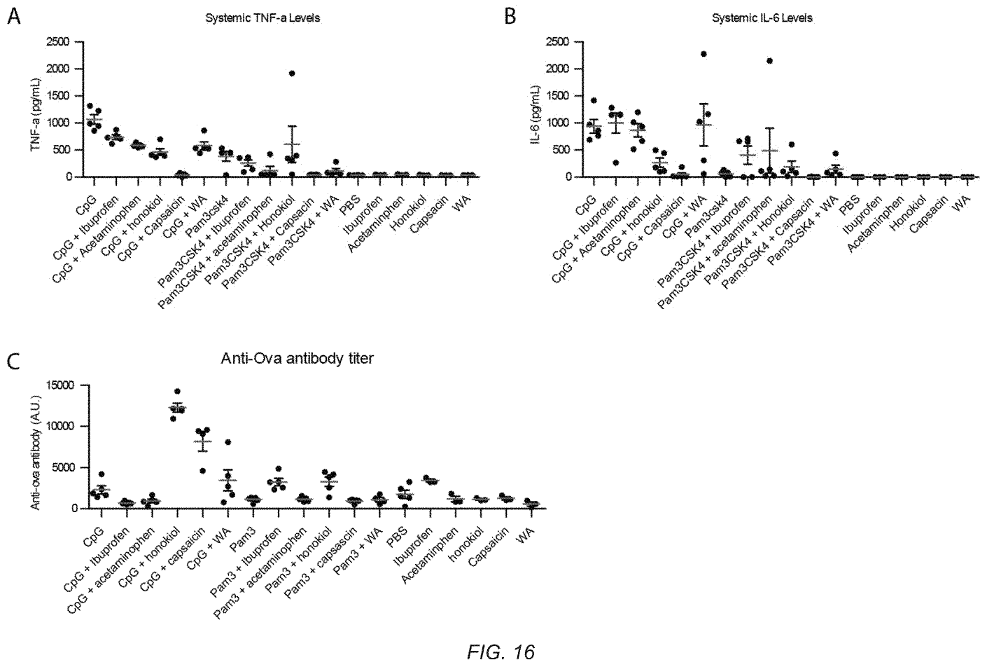

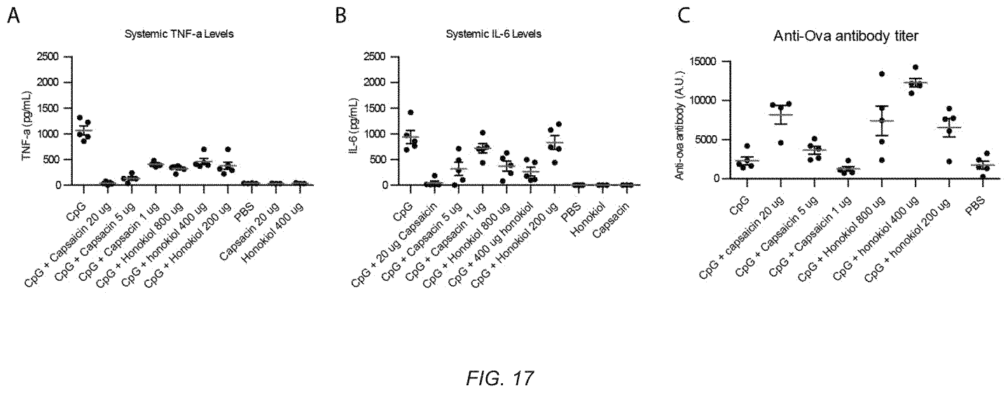

The inventors next wanted to examine how capsaicin, honokiol and WA would work with other TLR agonists. Additionally, the inventors wanted to compare these inhibitors to the most commonly used anti-inflammatory drugs, ibuprofen and acetaminophen. The inventors formulated the vaccines using AddaVax, an oil-in-water emulsion to ensure solubility of the inhibitors. The inventors used either CpG, a TLR 9 agonist or Pam3CSK4, a TLR 1/2 agonist as the immune adjuvant and ovalbumin as the antigen. The inventors analyzed systemic cytokines one hour post-vaccination. Vaccines formulated with NF-kB inhibitors demonstrated reduction of systemic TNF-a levels. Most notably, capsaicin levels were comparable to PBS alone (FIG. 16A). Ibuprofen, acetaminophen and WA were less effective at decreasing systemic IL-6 levels. Capsaicin decreased to levels consistent with PBS and honokiol showed a 4 fold decrease in systemic IL-6 (FIG. 16B). On day 21 the inventors analyzed the anti-ovalbumin antibody titer. Vaccines with CpG demonstrated higher antibody titers overall. Both acetaminophen and ibuprofen demonstrated decreases in antibody titer. CpG+honokiol demonstrated the highest antibody titer. Both acetaminophen and ibuprofen demonstrated decreases in antibody titer (FIG. 16C).

### Example 3

### Small Molecule NF-κB Inhibitors as Immune Potentiators for Enhancement of Vaccine Adjuvants

Adjuvants are added to vaccines to enhance the immune response and provide increased protection. In the last decade, hundreds of synthetic immune adjuvants have been created, but many induce undesirable levels of proinflammatory cytokines including TNF-α and IL-6. Here, the inventors present small molecule NF-κB inhibitors that can be used in combination with an immune adjuvant to both decrease markers associated with safety risks and improve the protective response of vaccination. Additionally, the inventors synthesized a library of honokiol derivatives identifying several promising candidates for use in vaccine formulations.

Vaccines remain one of the most effective ways of preventing disease. Despite their immense success in preventing diseases such as polio, tetanus, and small pox, diseases such as HIV and dengue present challenges that current clinical vaccine technologies cannot provide. To solve this problem, one strategy that has been explored is to include adjuvants, molecules that enhance the immune response. Although novel adjuvants generate higher quality immune responses than can be achieved with current approved adjuvants, to date, very few have been approved for use in human vaccines. This disconnect is due, in part, to the challenge of balancing the proinflammatory immune response with the protective, adaptive immune response. The inventors recently reported that vaccines could be improved through the use of a peptide NF-κB inhibitor, SN50 in combination with an immune adjuvant. The addition of SN50 to adjuvanted vaccines led to increased safety of the adjuvant while enhancing protection against disease. Although this method proved both general across a wide range of adjuvants and effective against antigens of a variety of diseases, it still required a large amount of the peptide to enable optimal safety and protection. Scale-up of peptides present synthetic challenges and can result in expensive production costs, limiting their potential in a clinical setting. Peptides might also induce an immune response against themselves leading to a potential for decreased enhancement in subsequent vaccinations. The inventors chose to explore other small molecule NF-κB inhibitors as immune potentiators to overcome these challenges.

Here the inventors demonstrate that select small molecule NF-κB inhibitors are effective at reducing adjuvant-induced inflammation while also increasing the adaptive immune response. At the same time, the inventors demonstrate that not all NF-κB inhibitors are effective immune potentiators. Of the molecules tested, honokiol and capsaicin proved to be effective at both limiting inflammation and potentiating the protective response. Through knockout studies, the inventors demonstrate that the increase in antigen specific antibodies is independent from the anti-inflammatory activity. To determine if these small molecules could be improved by chemical synthesis, the inventors explored derivatives of honokiol and found several promising candidates for potential use in vaccines.

H. Results and Discussion

1. Exploration of Small Molecule NF-κB Inhibitors In Vitro

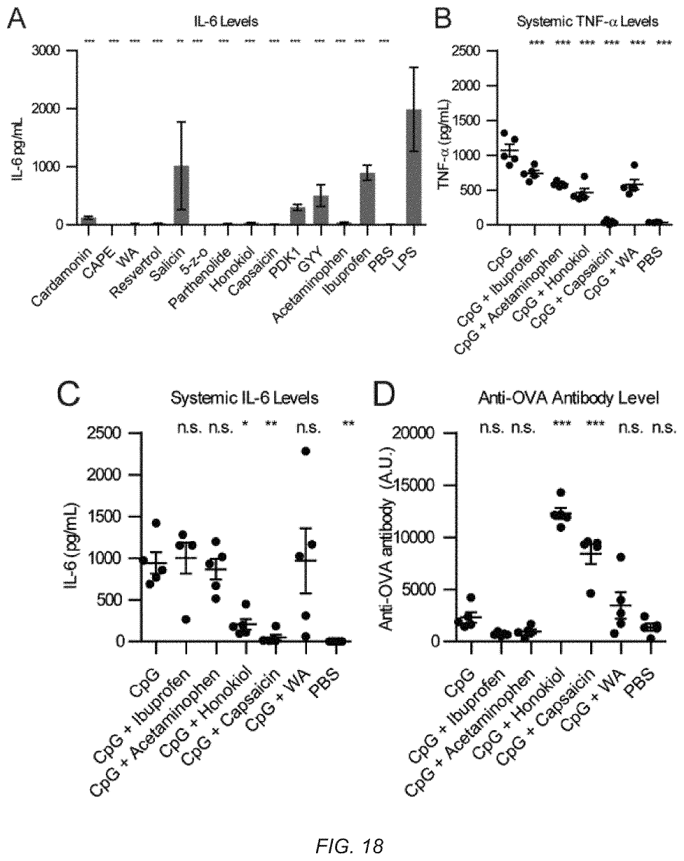

To begin exploring alternative NF-κB inhibitors, the inventors examined the literature for promising candidates. Due to the strong correlation between NF-KB activation and sepsis, cancer and autoimmune disorders, a large library of NF-κB inhibitors have been identified. The inventors first wanted to analyze the potential of a variety small molecule NF-κB inhibitors to inhibit inflammation in vitro in combination with lipopolysaccharide (LPS), a TLR4 agonist. The inventors chose several common commercially available NF-κB inhibitors and tested them in RAW macrophages. The inventors chose to examine: Cardamonin (40 μM), Caffeic acid phenethyl ester (CAPE) (100 μM), Withaferin A (WA) (400 nM), Resveratrol (10 μM), Salicin (100 nM), 5Z-7-Oxozeaenol (5-z-O) (5 μM), Parthenolide (20 μM), Honokiol (20 μM), Capsaicin (200 μM), PDK1/Akt/Flt dual pathway inhibitor (PDK1) (1 μM), and GYY 4137 (GYY) (200 μM). To determine if immune potentiation was specific to NFkB or general to all anti-inflammatory molecules, the inventors included the most common, FDA approved anti-inflammatory drugs acetaminophen (10 mM) and ibuprofen (800 μM). The inventors treated RAW macrophages with inhibitors and LPS and assayed the supernatant for IL-6 secretion (FIG. 18A).

2. Exploration of Small Molecule NF-κB Inhibitors In Vivo

The inventors next wanted to examine how these inhibitors would alter safety and protection in vivo. To test this in vivo, the inventors chose the small molecule inhibitors that were the most effective at inhibiting IL-6 expression in vitro, capsaicin, honokiol and withaferin A (WA) and ran them alongside acetaminophen and ibuprofen. The inventors chose to vaccinate mice using CpG, a TLR9 agonist. For the in vivo vaccination, the inventors used ovalbumin (OVA) as a model antigen to examine the changes in humoral response. The inventors vaccinated mice with 100 μg OVA, 50 μg CpG, and inhibitor (800 μg ibuprofen, 2 mg acetaminophen, 400 μg honokiol, 20 μg capsaicin or 600 μg WA). Due to the difficulty in solubility, all inhibitors were suspended in AddaVax, a squalene-based oil-in-water nano-emulsion, to enable effective vaccine suspensions. The inventors chose to analyze systemic levels of TNF-α and IL-6 because high levels of these cytokines are pyrogenic and have been correlated with undesirable vaccine-related side effects. Mice vaccinated with CpG demonstrated high levels of TNF-α (1067 pg/mL) (FIG. 18B). Addition of an NF-κB inhibitor decreased the level of TNF-α. Ibuprofen decreased to 738 pg/mL (1.4 fold), acetaminophen (1.8 fold), honokiol (2.3 fold), capsaicin (28 fold to background levels), and WA by 1.8 fold. The systemic levels of IL-6 were also high with CpG vaccination (941 pg/mL). The groups that included an NF-κB inhibitor did not always decrease the level of IL-6 (FIG. 18C). Ibuprofen, acetaminophen and WA did not decrease IL-6 expression significantly. However, honokiol and capsaicin dramatically reduced the systemic levels of IL-6 to 266 pg/mL (3.5 fold) and 47.4 pg/mL (20 fold), respectively.

On day 21, the inventors analyzed the anti-OVA antibody levels (FIG. 18D). CpG was 1.3 fold more than PBS. Ibuprofen and acetaminophen were 3.2 and 2.4 fold lower that CpG alone. CpG+honokiol was 5.3 fold more than CpG alone. CpG+capsaicin was 3.5 fold higher than CpG alone. CpG+WA was 1.5 fold lower than CpG alone.

3. Dose-Dependence of Capsaicin and Honokiol

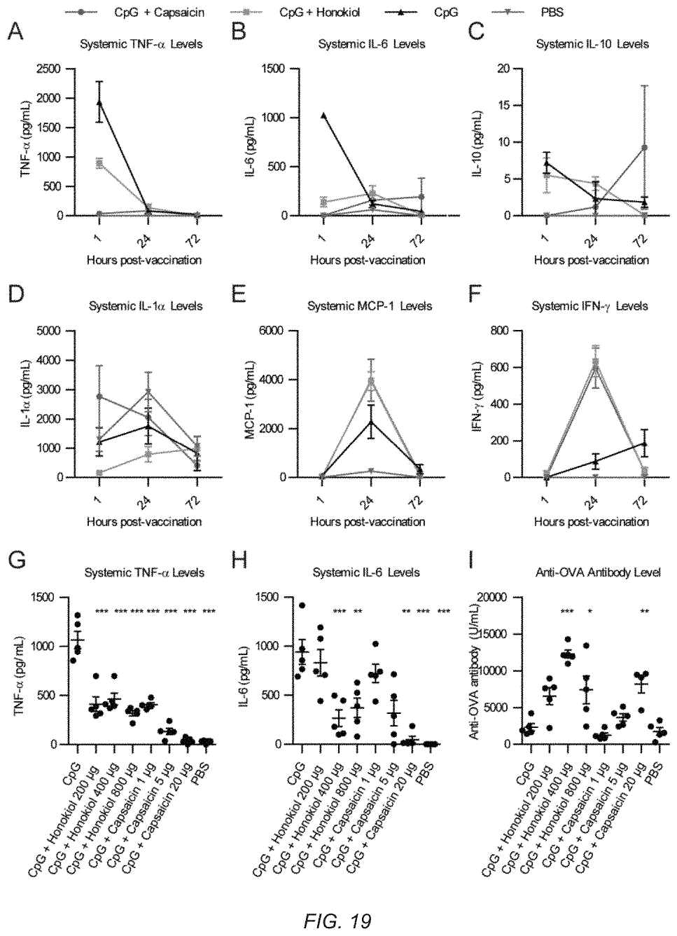

Of the candidates, capsaicin and honokiol demonstrated exceptional promise in these studies so the inventors examined them further. To better understand how these molecules were altering the immune response, the inventors vaccinated mice as described above and analyzed a larger variety of cytokines at a various timepoints to understand how honokiol and capsaicin alter the expression over the period of 72 hrs post administration. The inventors analyzed 13 cytokines: IL-1α, IL-1β, IL-6, IL-10, IL-12p70, IL-17A, IL-23, IL-27, MCP-1, IFN-β, IFN-γ, TNF-α, and GM-CSF. Of these, only 6 cytokines had detectable levels in the assay: TNF-α, IL-6, IL-10, IL-1α, MCP-1 and IFN-γ (FIG. 19A-F). Consistent with previous findings, CpG induced TNF-α and IL-6 expression peaked at 1 h. Interestingly, CpG combined with either capsaicin or honokiol had increased IFN-γ levels at 24 h compared to CpG alone and slightly elevated MCP-1 levels, demonstrating that both capsaicin and honokiol are acting to potentiate the immune response and are not simply repressing immune activation. The inventors next wanted to understand how changing the dose would alter innate and adaptive immune responses. For honokiol, the inventors tested a concentration 2-fold higher (800 μg) and 2-fold lower (200 μg) than the original dose (400 μg). A pain response was observed in mice vaccinated with the original dose of capsaicin (20 μg), so the inventors wanted to examine if the dose could be lowered while maintaining adequate anti-inflammatory activity and antibody-boosting potential. The inventors chose to test a dose 4-fold lower (5 μg) and 20-fold lower (1 μg) than the original dose (20 μg). All doses of honokiol demonstrated a significant decrease in TNF-α expression compared to CpG alone, however there was no significant difference between the different doses (FIG. 19G). Capsaicin decreased TNF-α levels significantly across all doses compared to CpG alone. Doses of 5 μg and 20 μg decreased levels of TNF-α significantly more than 1 μg (FIG. 19G). The level of IL-6 was only decreased with 400 μg and 800 μg honokiol and 20 μg capsaicin (FIG. 19H). Twenty-one days later, the inventors analyzed differences in anti-OVA antibody level and found that all doses of honokiol increased levels of anti-OVA antibodies compared to CpG alone and the highest level was found with 400 μg honokiol (FIG. 2I). 1 μg and 5 μg of capsaicin did not change level of anti-OVA antibodies in the serum compared to CpG alone, however 20 μg significantly increased serum levels.

4. Determining the TRPV1-Mediated Effects of Capsaicin

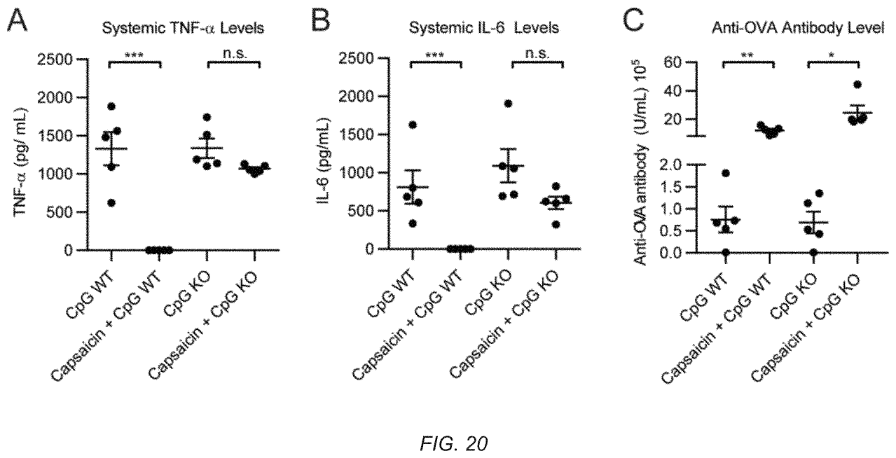

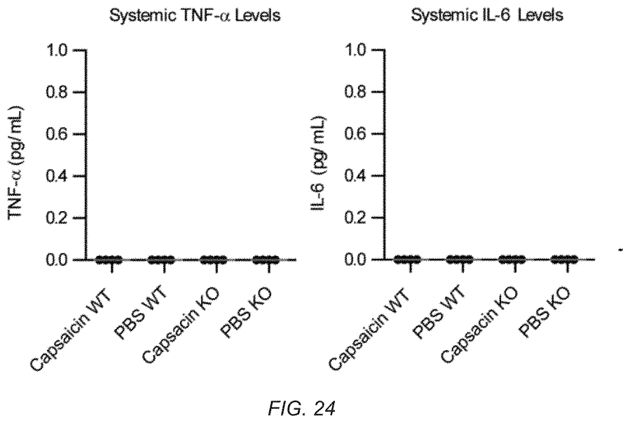

The primary in vivo target for capsaicin is the transient receptor potential cation channel subfamily V member 1 (TRPV1). TRPV1 modulates the immune response in a variety of ways, and importantly, has been implicated in dampening systemic inflammation associated with sepsis. However, it has never been explored in a vaccine setting. To understand how activation of TRPV1 may be modulating the effects of the adjuvant, the inventors compared the immediate inflammatory response of the vaccination in wild type mice (WT) and TRPV1 knockout mice. The inventors vaccinated WT and TRPV1 KO mice with 100 μg OVA and: 50 μg CpG, 50 μg CpG+20 μg capsaicin or PBS. The inventors analyzed systemic levels of TNF-α and IL-6 1 h after vaccination. The inventors found that CpG induced high levels of TNF-α and IL-6 in both WT and TRPV1 KO mice. Addition of capsaicin dramatically and significantly reduced both TNF-α levels and IL-6 levels in the WT mice (FIGS. 20A, 20B, 24). Although the mean was slightly lower for both TNF-α and IL-6 in the TRPV1 KO mice, these differences were not statistically significant. This demonstrated that TRPV1 activation is responsible for the capsaicin-induced decrease in systemic cytokine levels. To examine if the increased antibody level was due to TRPV1 activation on day 21, the inventors analyzed levels of anti-OVA antibodies in the serum (FIGS. 20C, 24). Interestingly, the inventors found that anti-OVA antibody levels were increased in groups with capsaicin+CpG in both WT and KO mice. This implies that the antibody-boosting activity of capsaicin is separate from TRPV1-dependent decrease in inflammatory cytokines. This result demonstrates both that the decrease in inflammation is not responsible for the antibody-boosting activity of the NF-κB inhibitor a result that the inventors demonstrated previously, and also that the enhancement of the adaptive response is TRPV1 independent. These results, while not definitive, showed two separate, but correlated mechanisms for capsaicin which result in the reduction in cytokines and increase antibodies. As such, capsaicin did not warrant further examination as a potential clinical immune potentiator. The inventors will explore the mechanistic implications of this for immune potentiators more broadly in future publications.

5. Synthesis of Honokiol Derivative Library

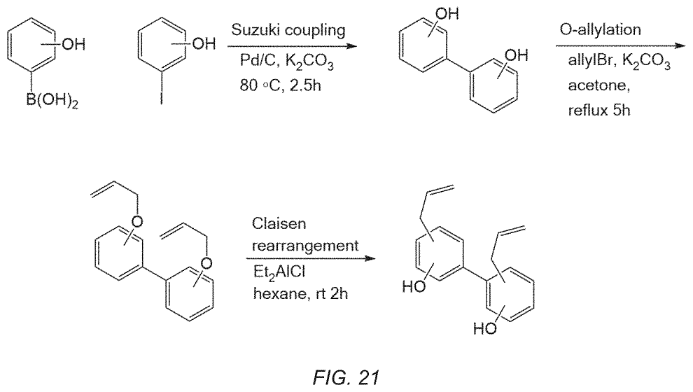

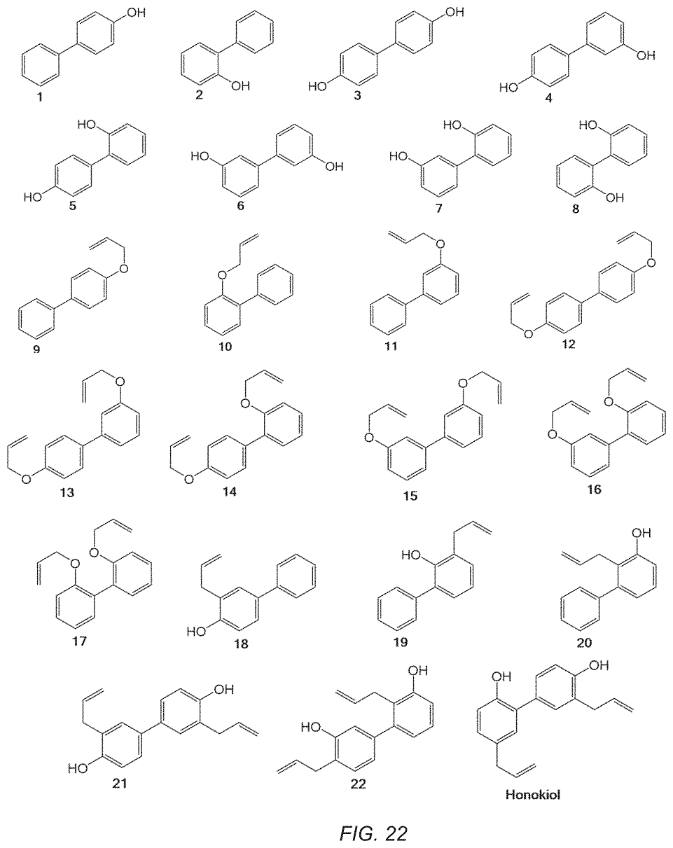

With capsaicin possessing two parallel mechanisms and possessing well-established side effects, the inventors explored honokiol for further development as a candidate. An important question for immune potentiators and honokiol was if standard SAR methods would yield alteration in potentiation activity. To further explore this idea, the inventors synthesized a library of di-aryl derivatives based on honokiol's structure. Honokiol derivative libraries have been synthesized previously and examined for their effects on neuroprotection⁶⁰, antimicrobial agents⁶¹ and anti-cancer⁶² among others.^{63,64} However, to date no such study has examined the effects of honokiol analogs on vaccines or a combination of anti-inflammatory activity and adaptive immune response. Phenylphenols and biphenols were prepared according to the reaction scheme depicted in FIG. 21 using Pd-catalyzed Suzuki coupling using corresponding iodophenols and hydroxyphenylboronoic acids as starting materials. These compounds were O-allylated using allyl bromide. The resulting compounds were subjected to Claisen rearrangement using diethyl aluminum chloride to yield a variety of ring substitutions (FIG. 21).

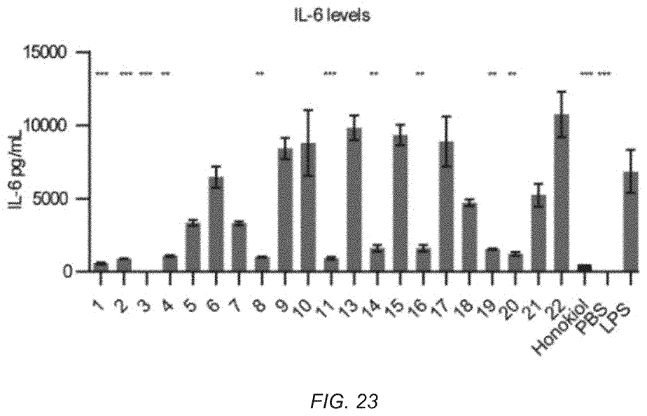

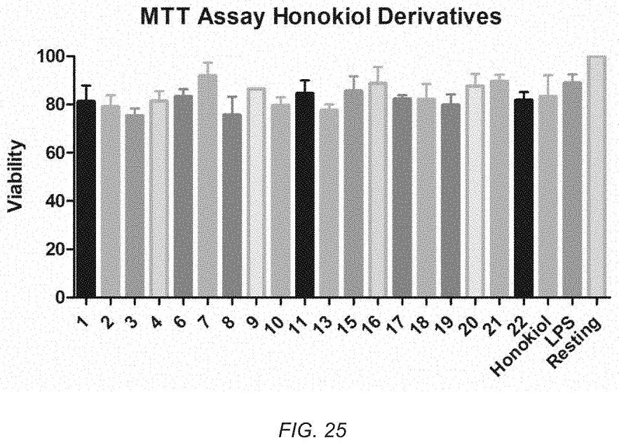

The inventors examined the activities of the synthesized compounds in order to understand how the various functional groups affect anti-inflammatory action or adaptive immune response (FIGS. 23, 25). The inventors treated RAW macrophages with honokiol derivatives and LPS and analyzed IL-6 expression. The addition of LPS alone without a honokiol derivative gave high levels of IL-6 expression (6848 pg/mL). The addition of honokiol decreased IL-6 levels to 260 pg/mL, a decrease of 26-fold. Several derivatives including compounds: 1, 2, 3, 4, 8, and 11 demonstrated similar reductions in IL-6 expression.

I. Conclusion

In summary, the inventors present that select small molecule inhibitors of NF-κB can decrease the inflammatory effects of adjuvanted vaccination—potentially enabling safer vaccination while also acting as immune potentiators and increasing the antibody level. The inventors identified two such immune potentiators, honokiol and capsaicin that effectively decrease inflammation while increasing the adaptive response. The inventors additionally provide evidence that implies that the decrease in inflammation is separate from the increase in antibody response, potentially enabling distinct tunability of either response. This study also identifies that only select NF-κB inhibitor can be used as immune potentiators, this broadens the potential for further modulation of the immune response. The inventors additionally examined a library of honokiol derivatives and found that several honokiol derivatives are promising candidates for future testing in vivo. In conclusion, the inventors have demonstrated that using small molecule NF-κB inhibitors in combination with common immune adjuvants can decrease the production of pro-inflammatory cytokines TNF-α and IL-6 while boosting antibody levels.

J. Materials and Methods

1. In Vitro Assays

RAW macrophage cytokine analysis: RAW 264.7 macrophages were passaged and plated in a cell culture treated 12-well plate at 0.5×10⁶ cells/well in 1 mL DMEM containing 10% FBS. Cells were grown for 2 days. Media was exchanged for 1 mL DMEM containing 10% HIFBS. Inhibitors were added at indicated concentrations and incubated for 45 min. After 45 min, LPS was added at 100 ng/mL and incubated at 37° C. and 5% CO₂ for 24 h. Cell supernatant was removed and analyzed using BD Cytometric Bead Array Mouse Inflammation Kit.

2. In Vivo Assays

All animal procedures were performed under a protocol approved by the University of Chicago Institutional Animal Care and Use Committee (IACUC). 6-8 week-old C57/B6 female mice were purchased from Jackson Laboratory (JAX). 6-8 week-old C57/B6 female Trpv1^{tmlJu} mice were purchased from JAX for TRPV1 KO experiment. All compounds were tested for endotoxin prior to use. All vaccinations were administered intramuscularly in the hind leg. Blood was collected from the sapheneous vein at time points indicated.

Antigens were purchased from Invitrogen (Vaccigrade Ovalbumin). Vaccigrade CpG ODN 1826 was purchased from Adipogen. AddaVax™ was purchased from Invivogen.

Vaccination: Mice were lightly anesthetized with isoflurane and injected intramuscularly in the hind leg with 50 μL containing ovalbumin (100 μg), adjuvant, inhibitor and PBS. Adjuvant doses: CpG, 50 μg. Inhibitor concentrations: Honokiol (400 μg), Capsaicin (20 μg), Withaferin A (600 μg), acetaminophen (2 mg), ibuprofen (800 μg). All vaccines contained 25 μL AddaVax™ to enhance solubility.

Plasma cytokine analysis: Blood was collected from mice at time points indicated in 0.2 mL heparin coated collection tubes (VWR Scientific). Serum was isolated via centrifugation 2000×g for 5 min. Supernatant was collected and stored at −80° C. until use. Serum was analyzed using BD Cytometric Bead Array Mouse Inflammation cytokine kit or LEGENDplex™ Mouse Inflammation Panel (Biolegend) according to manufacturer's protocol.

Antibody quantification: Mice were vaccinated with indicated formulations. Blood was collected at time points indicated in 0.2 mL heparin coated collection tubes (VWR Scientific) for plasma or uncoated tubes for serum. Plasma was isolated via centrifugation (2000×g, 5 min). Serum was isolated by allowing blood to clot for 15-30 min RT and centrifuging (2000×g for 10 min) at 4° C. Serum was analyzed using a quantitative anti-ovalbumin total Ig's ELISA kit (Alpha Diagnostic International) according to the specified protocol. Data was analyzed using Graphpad Prism.

3. Chemistry

Conditions for Suzuki Coupling: Hydroxyphenol boronic acid (20 mmol) was dissolved in 100 mL water. Appropriate iodophenol (10 mmol) and K₂CO₃ (40 mmol) was added followed by Pd/C (2 mol %). Solution heated to 80 C for 3 h. Solution was acidified with 1M HCl and extracted with EtoAc and washed with brine. Solvent evaporated in vacuo. Compound was purified by column chromatography.

Conditions for O-allylations: Phenol (1 mmol) (Derivative 1-?) was dissolved in dry acetone (5 mL) and K₂CO₃ (2 mmol) added. AllylBr was added dropwise and refluxed. Reaction was monitored by TLC until completion (5-12 h). Reaction mixture was cooled and volatiles were removed in vacuo. 10% NaOH was added to the mixture and extraction was performed using ethyl acetate, washed with brine and organic layers dried using MgSO₄. Solvent was removed in vacuo affording an oily material that was purified by column chromatography to yield the O-allylated derivative.

Conditions for Claisen rearrangement: O-allylated derivatives (1 mmol) were dissolved in dry hexane (10 mL). Et₂AlCl in dry hexane (4 mL) was added dropwise under argon. Mixture was stirred at room temperature for 2 h. The mixture was cooled on an ice bath and quenched using 2M HCl (20 mL). Extraction was performed with EtOAc, washed with brine and dried over MgSO₄. Solvent was removed in vacuo affording an oily material that was purified by column chromatography to yield the C-allyl derivative.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

1. Petrovsky, N. & Aguilar, J. C. Vaccine adjuvants: Current state and future trends. Immunol. Cell Biol. 82, 488-496 (2004).

2. Coffman, R. L., Sher, A. & Seder, R. A. Vaccine Adjuvants: Putting Innate Immunity to Work. Immunity 33, 492-503 (2010). 3. Pashine, A., Valiante, N. M. & Ulmer, J. B. Targeting the innate immune response with improved vaccine adjuvants. Nat. Med. 11, S63-S68 (2005).

4. Hoebe, K., Janssen, E. & Beutler, B. The interface between innate and adaptive immunity. Nature Immunology (2004). doi:10.1038/ni1004-971

5. D'Agostino, P. M. et al. Viral-Induced Encephalitis Initiates Distinct and Functional CD103+ CD11b+ Brain Dendritic Cell Populations Within the Olfactory Bulb. Proc. Natl. Acad. Sci. 109, 6175-6180 (2012).

6. Banchereau, J. & Steinman, R. M. Dendritic cells and the control of immunity. Nature 392, 245-252 (1998).

7. van Duin, D., Medzhitov, R. & Shaw, A. C. Triggering TLR signaling in vaccination. Trends Immunol. 27, 49-55 (2006).

8. Dale, C. J. et al. Evaluation in macaques of HIV-1 DNA vaccines containing primate CpG motifs and fowlpoxvirus vaccines co-expressing IFNγ or IL-12. Vaccine 23, 188-197 (2004).

9. Weeratna, R., Comanita, L. & Davis, H. L. CPG ODN allows lower dose of antigen against hepatitis B surface antigen in BALB/c mice. Immunol. Cell Biol. 81, 59-62 (2003).

10. Klinman, D. M., Xie, H. & Ivins, B. E. CpG oligonucleotides improve the protective immune response induced by the licensed anthrax vaccine. Ann. N. Y. Acad. Sci. 1082, 137-150 (2006).

11. Davalia, E.; Kennedy, R.; Celis E. Generation of antitumor immunity by cytotoxic T lymphocyte epitope peptide vaccination, CpG-oligodeoxynucleotide adjuvant, and CTLA-4 blockade.—Semantic Scholar. Cancer Res. 63(12), 3281-8 (2003).

12. Krieg, A. M. Toll-like receptor 9 (TLR9) agonists in the treatment of cancer. Oncogene 27, 161-167 (2008).

13. Bode, C., Zhao, G., Steinhagen, F., Kinjo, T. & Klinman, D. M. CpG DNA as a vaccine adjuvant. Expert Rev. Vaccines 10, 499-511 (2011).

14. Klinman, D. CpG DNA as a vaccine adjuvant: Expert Review of Vaccines Expert Review of Vaccines 2(2), 305-315 (2003).

15. Kanzler, H., Barrat, F. J., Hessel, E. M. & Coffman, R. L. Therapeutic targeting of innate immunity with Toll-like receptor agonists and antagonists. Nat. Med. 13, 552-559 (2007).

16. Mbow, M. L., De Gregorio, E., Valiante, N. M. & Rappuoli, R. New adjuvants for human vaccines. Curr. Opin. Immunol. 22, 411-416 (2010).

17. Christian, L. M., Porter, K., Karlsson, E. & Schultz-Cherry, S. Proinflammatory cytokine responses correspond with subjective side effects after influenza virus vaccination. Vaccine 33, 3360-3366 (2015).

18. Simon, W. L., Salk, H. M., Ovsyannikova, I. G., Kennedy, R. B. & Poland, G. A. Cytokine production associated with smallpox vaccine responses. Immunotherapy 6, 1097-1112 (2014).

19. Pasquale, A. D., Preiss, S., Silva, F. T. D. & Garcon, N. Vaccine adjuvants: from 1920 to 2015 and beyond. Vaccines 3, 320-343 (2015).

20. Barton, G. M. & Medzhitov, R. Toll-Like Receptor Signaling Pathways. Science 300, 1524-1525 (2003).

21. Lawrence, T. The Nuclear Factor NF-B Pathway in Inflammation. Cold Spring Harb. Perspect. Biol. 1, a001651-a001651 (2009).

22. Akira, S. & Takeda, K. Toll-like receptor signalling. Nat. Rev. Immunol. 4, 499-511 (2004).

23. Hoffmann, A., Leung, T. H. & Baltimore, D. Genetic analysis of NF-κB/Rel transcription factors defines functional specificities. EMBO 1 22, 5530-5539 (2003).

24. Hoffmann, A. & Baltimore, D. Circuitry of nuclear factor kappaB signaling. Immunol. Rev. 210, 171-186 (2006).

25. Baeuerle, P. A. & Henkel, T. Function and Activation of NF-kappaB in the Immune System. Annu. Rev. Immunol. 12, 141-179 (1994).

26. Dolcet, X., Llobet, D., Pallares, J. & Matias-Guiu, X. NF-kB in development and progression of human cancer. Virchows Arch. 446, 475-482 (2005).

27. Wang, C.-Y., Mayo, M. W. & Baldwin, A. S. TNF- and Cancer Therapy-Induced Apoptosis: Potentiation by Inhibition of NF-κB. Science 274, 784-787 (1996).

28. Xia, Y., Shen, S. & Verma, I. M. NF-κB, an active player in human cancers. Cancer Immunol. Res. 2, 823-830 (2014).

29. Erstad, D. J. & Cusack, J. C. Targeting the NF-κB pathway in cancer therapy. Surg. Oncol. Clin. N. Am. 22, 705-746 (2013).

30. Bacher, S. & Schmitz, M. L. The NF-kB Pathway as a Potential Target for Autoimmune Disease Therapy. Curr. Pharm. Des. 10(23), 2827-37 (2004).

31. Palanki, M. s s. Inhibitors of AP-1 and NF-kB Mediated Transcriptional Activation: Therapeutic Potential in Autoimmune Diseases and Structural Diversity. Curr. Med. Chem. 9(2) 219-227 (2002).

32. Letoha, T. et al. In Vitro and in Vivo Nuclear Factor-κB Inhibitory Effects of the Cell-Penetrating Penetratin Peptide. Mol. Pharmacol. 69, 2027-2036 (2006).

33. Liu, S. F., Ye, X. & Malik, A. B. In vivo inhibition of nuclear factor-kappa B activation prevents inducible nitric oxide synthase expression and systemic hypotension in a rat model of septic shock. J. Immunol. 159, 3976-3983 (1997).

34. Eigler, A., Sinha, B., Hartmann, G. & Endres, S. Taming TNF: strategies to restrain this proinflammatory cytokine. Immunol. Today 18, 487-492 (1997).

35. Tak, P. P. & Firestein, G. S. NF-κB: a key role in inflammatory diseases. J. Clin. Invest. 107, 7-11 (2001).

36. Wang, J. et al. Distinct Roles of Different NF-κB Subunits in Regulating Inflammatory and T Cell Stimulatory Gene Expression in Dendritic Cells. J. Immunol. 178, 6777-6788 (2007).

37. Zienkiewicz, J.; Armitage, A.; Hawiger, J. Targeting Nuclear Import Shuttles, Importins/Karyopherins alpha by a Peptide Mimicking the NFkB1/p50 Nuclear Localization Sequence. J. Am. Heart Assoc. 2(5), e000386 (2013).

38. Netea, M. G., Kullberg, B. J., Meer, V. der & M, J. W. Circulating Cytokines as Mediators of Fever. Clin. Infect. Dis. 31, S178-S184 (2000).

39. Bouvier, N. M. & Lowen, A. C. Animal Models for Influenza Virus Pathogenesis and Transmission. Viruses 2, 1530-1563 (2010).

40. Daniele, G. et al. The inflammatory status score including IL-6, TNF-α, osteopontin, fractalkine, MCP-1 and adiponectin underlies whole-body insulin resistance and hyperglycemia in type 2 diabetes mellitus. Acta Diabetol. 51, 123-131 (2014).

41. Segev, G., Kass, P. H., Francey, T. & Cowgill, L. D. A Novel Clinical Scoring System for Outcome Prediction in Dogs with Acute Kidney Injury Managed by Hemodialysis. J. Vet. Intern. Med. 22, 301-308 (2008).

42. Page, K. M. Zhang, L.; Medizabal, A.; Wease, S.; Carter, S.; Shoulars, K.; Gentry, T.; Balber, A.; Kurtzberg, J. The Cord Blood Apgar: a novel scoring system to optimize selection of banked cord blood grafts for transplantation (CME). Transfusion (Paris) 52, 272-283 (2012).

43. Ramachandran, A.; Snehalatha, C.; Baskar, A.; Mary, S.; Kumar, C.; Selvam, S.; Catherine, A.; Viay, V. Temporal changes in prevalence of diabetes and impaired glucose tolerance associated with lifestyle transition occurring in the rural population in India. Diabetologia 47, 860-865 (2004).

44. Bhardwaj, N., Gnjatic, S. & Sawhney, N. B. TLR AGONISTS: Are They Good Adjuvants? Cancer J. Sudbury Mass 16, 382-391 (2010).

45. Gilmore, T.; Herscovitch, M. Inhibitors of NF-kappaB signaling: 785 and counting. Oncogene 23(51), 6887-99 (2006).

46. Huang, J. et al. Broad and potent HIV-1 neutralization by a human antibody that binds the gp41-gp120 interface. Nature 515, 138-142 (2014).

47. Audibert, F. M. & Lise, L. D. Adjuvants: current status, clinical perspectives and future prospects. Trends in Pharmacological Sciences 14, 174-178 (1993).

48. Tom, J. K. et al. Applications of Immunomodulatory Immune Synergies to Adjuvant Discovery and Vaccine Development. Trends in Biotechnology 37, 373-388 (2019). 49. Immune potentiator for increased safety and improved protection of vaccines by NF-kB|modulation bioRxiv. Available at: https://www.biorxiv.org/content/10.1101/489732v1. (Accessed: 17 Mar. 2019)

50. Lau, J. L. & Dunn, M. K. Therapeutic peptides: Historical perspectives, current development trends, and future directions. Bioorganic & Medicinal Chemistry 26, 2700-2707 (2018).

51. Otvos, L. & Wade, J. D. Current challenges in peptide-based drug discovery. Front Chem 2, (2014).

52. Advances in understanding sepsis European Journal of Anaesthesiology Cambridge Core. Available at: https://www.cambridge.org/core/journals/europeanjournal-of-anaesthesiology/article/advances-in-understanding-sepsis/28EBD8952983DA23A573B97AF1C1A750. (Accessed: 17 Mar. 2019)

53. Scheuren, N., Bang, H., Munster, T., Brune, K. & Pahl, A. Modulation of transcription factor NF-κB by enantiomers of the nonsteroidal drug ibuprofen. British Journal of Pharmacology 123, 645-652 (1998).

54. Boulares, A. H., Giardina, C., Inan, M. S., Khairallah, E. A. & Cohen, S. D. Acetaminophen inhibits NF-kappaB activation by interfering with the oxidant signal in murine Hepa 1-6 cells. Toxicol. Sci. 55, 370-375 (2000).

55. Brito, R., Sheth, S., Mukherjea, D., Rybak, L. P. & Ramkumar, V. TRPV1: A Potential Drug Target for Treating Various Diseases. Cells 3, 517-545 (2014).

56. Wang, Y. & Wang, D. H. TRPV1 Ablation Aggravates Inflammatory Responses and Organ Damage during Endotoxic Shock. Clin. Vaccine Immunol. 20, 1008-1015 (2013).

57. Toledo-Mauriño, J. J. et al. The Transient Receptor Potential Vanilloid 1 Is Associated with Active Inflammation in Ulcerative Colitis. Mediators of Inflammation (2018). doi:10.1155/2018/6570371

58. Bodkin, J. V. & Fernandes, E. S. TRPV1 and SP: key elements for sepsis outcome? Br J Pharmacol 170, 1279-1292 (2013).

59. Fernandes, E. S. et al. TRPV1 deletion enhances local inflammation and accelerates the onset of systemic inflammatory response syndrome. J. Immunol. 188, 5741-5751 (2012).

60. Tripathi, S., Chan, M.-H. & Chen, C. An expedient synthesis of honokiol and its analogues as potential neuropreventive agents. Bioorganic & Medicinal Chemistry Letters 22, 216-221 (2012).

61. Kim, Y.-S. et al. Synthesis and microbiological evaluation of honokiol derivatives as new antimicrobial agents. Arch. Pharm. Res. 33, 61-65 (2010).

62. Sánchez-Penis, M., Murga, J., Falomir, E., Carda, M. & Marco, J. A. Synthesis of honokiol analogues and evaluation of their modulating action on VEGF protein secretion and telomerase-related gene expressions. Chem Biol Drug Des 89, 577-584 (2017).

63. Shen, J.-L. et al. Honokiol and magnolol as multifunctional antioxidative molecules for dermatologic disorders. Molecules 15, 6452-6465 (2010).

64. Lee, Y.-J. et al. Therapeutic applications of compounds in the Magnolia family. Pharmacol. Ther. 130, 157-176 (2011).

1. A method for vaccinating a subject comprising administering an NFkB inhibitor and an adjuvant to the subject.

2. (canceled)

3. The method of claim 1, wherein the NFkB inhibitor is SN50, capsaicin, or a compound of formula (I) 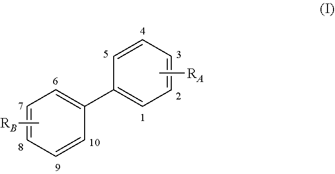
where:
RA is attached to one or more ring atoms at positions 1, 2, 3, 4, and 5, and each RA is independently hydrogen, hydroxyl, alkoxy, alkenoxy, or alkenyl; and
RB is attached to one or more ring atoms at positions 6, 7, 8, 9, and 10, and each RB is independently hydrogen, hydroxyl, alkoxy, alkenoxy, or alkenyl.

4. The method of claim 3, wherein the NFkB inhibitor is a compound of formula (I), wherein the NFkB inhibitor is further defined as

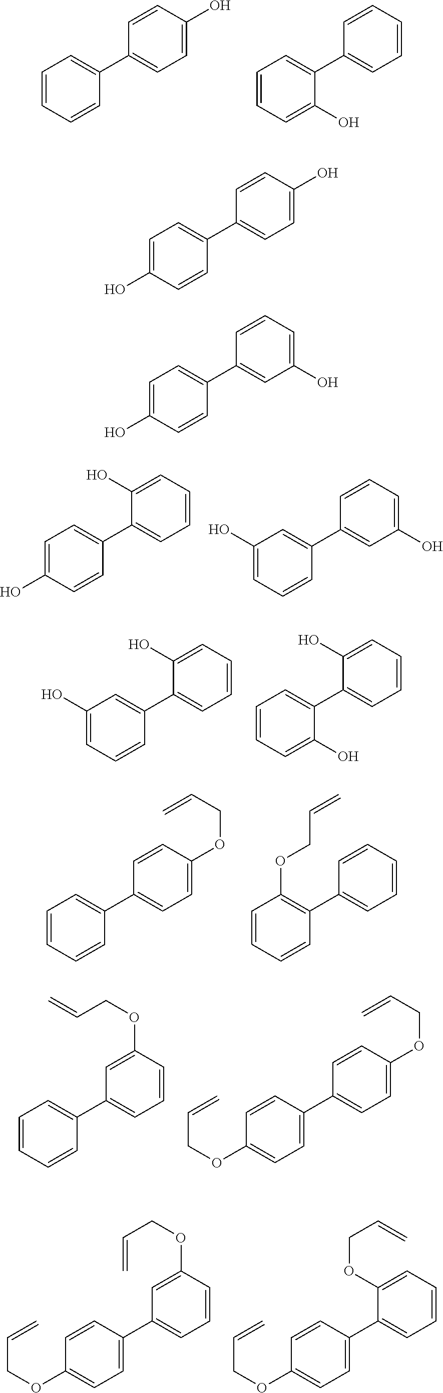

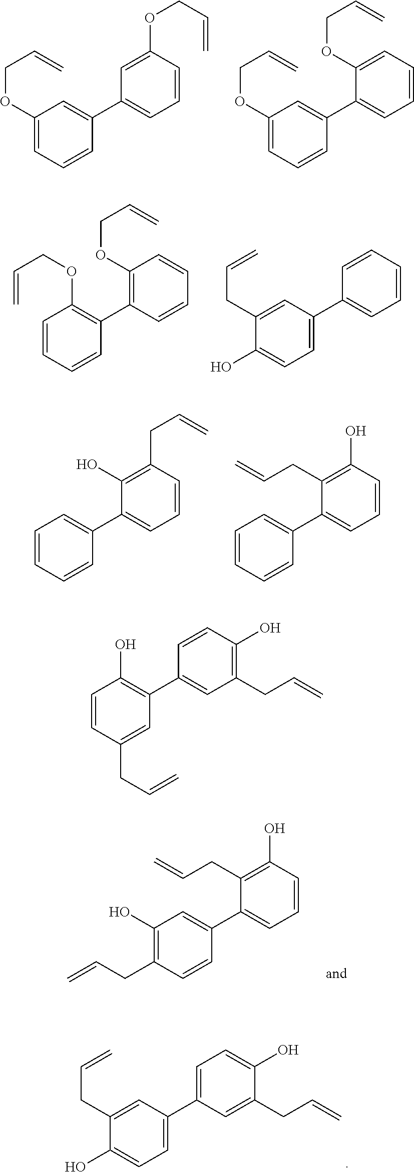

5. (canceled)

6. The method of claim 5, wherein the adjuvant is a toll-like receptor (TLR) agonist.

7. The method of claim 1, wherein the adjuvant is CpG, AddaVax, LPS, CpG ODN 1018, MPLA, Pam3CSK4, Pam2CSK4, R848, 2BXy, QS-21, AS01B, or Freund's complete adjuvant.

8. The method of claim 1, wherein the NFkB inhibitor is SN50 and the adjuvant is a TLR9 agonist.

9. The method of claim 1, wherein the method further comprises administration of one or more antigens.

10. The method of claim 1, wherein the method further comprises administration of inactivated virus, live attenuated virus, or antigenic fragments thereof.

11. (canceled)

12. The method of claim 10, wherein the method comprises administration of a dengue antigen.

13. (canceled)

14. The method of claim 10, wherein the method comprises administration of a HIV antigen.

15. (canceled)

16. (canceled)

17. The method of claim 1, wherein the NFkB inhibitor is administered prior to administration of the adjuvant.

18.-20. (canceled)

21. The method of claim 1, wherein the NFkB inhibitor and the adjuvant are administered in the same composition to the subject.

22. The method of claim 1, wherein at least 12 mg of the NFkB inhibitor is administered to the subject.

23.-25. (canceled)

26. The method of claim 1, wherein the subject has previously been administered an adjuvant.

27. (canceled)

28. A pharmaceutical composition comprising an NFkB inhibitor and an adjuvant.

29. The composition of claim 28, wherein the NFkB inhibitor is SN50, capsaicin, or a compound of formula (I) 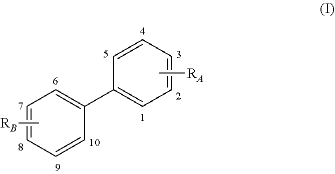
where:
R_{A} is attached to one or more ring atoms at positions 1, 2, 3, 4, and 5, and each R_{A} is independently hydrogen, hydroxyl, alkoxy, alkenoxy, or alkenyl; and
R_{B} is attached to one or more ring atoms at positions 6, 7, 8, 9, and 10, and each R_{B} is independently hydrogen, hydroxyl, alkoxy, alkenoxy, or alkenyl.

30. The composition of claim 29, wherein the NFkB inhibitor is a compound of formula (I), wherein the NFkB inhibitor is further defined as

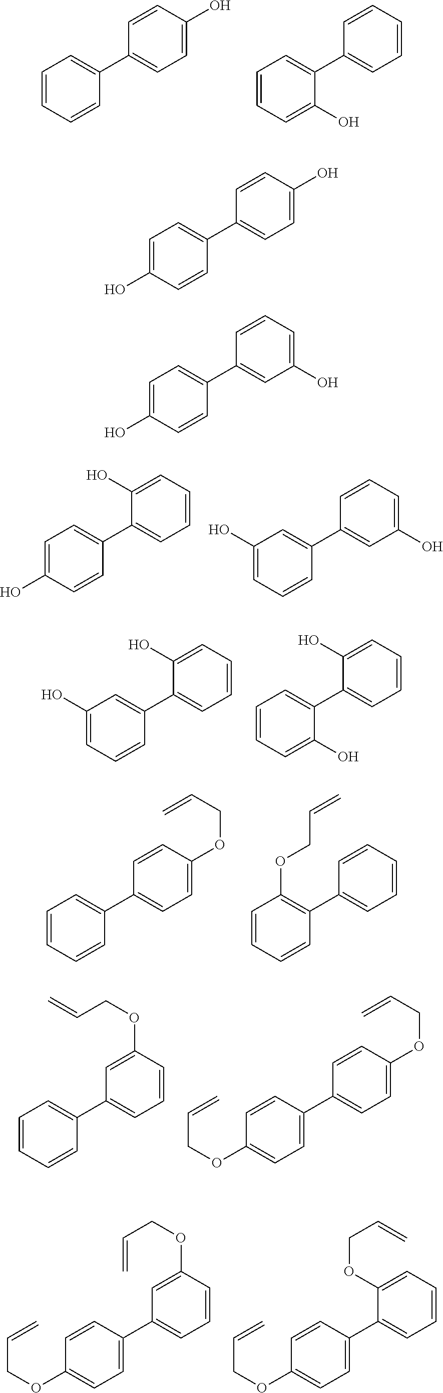

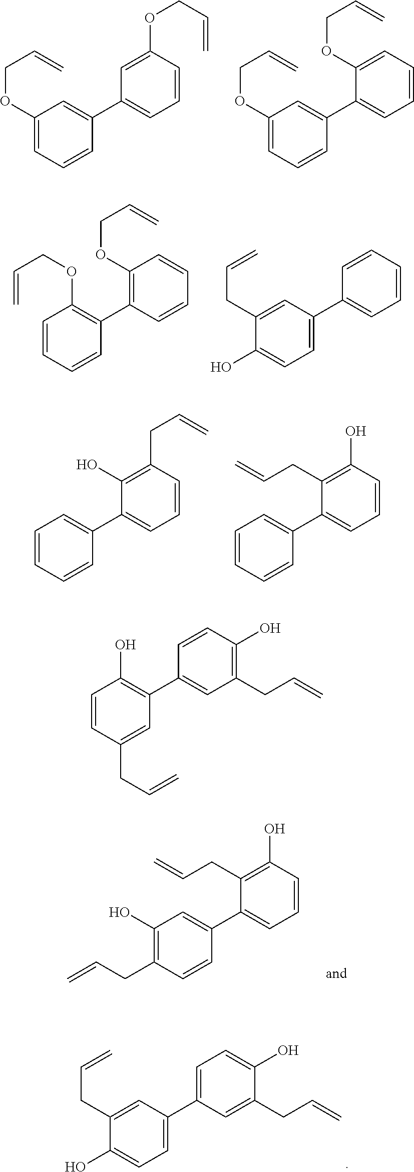

31.-47. (canceled)

48. A method for vaccinating a subject comprising administering the composition of claim 28 to the subject.

49. (canceled)

50. The method of claim 9, wherein the one or more antigens comprise a viral antigen, bacterial antigen, fungal antigen, parasite antigen, tumor antigen, or an antigen involved in an autoimmune disease.

51. The method of claim 50, wherein the one or more antigens comprise a tumor antigen.
